(19) United States
(12) Patent Application Publication
(10) Pub. No.: US 2010/0016274 A1
(43) Pub. Date: Jan. 21, 2010

(54) BETA-LACTAM CANNABINOID RECEPTOR MODULATORS

(76) Inventors: Gary A. Koppel, Indiannapolis, IN (US); Michael O. Chaney, Carmel, IN (US)

Correspondence Address: BARNES & THORNBURG LLP, 11 SOUTH MERIDIAN, INDIANAPOLIS, IN, 46204, US

(21) Appl. No.: 12/441,231

(22) PCT Filed: Sep. 14, 2007
(86) PCT No.: PCT/US07/78451

§ 371 Date: Sep. 3, 2009

Related U.S. Application Data
(60) Provisional application No. 60/825,669, filed on Sep. 14, 2006.
(60) Provisional application No. 60/825,673, filed on Sep. 14, 2006.

(51) Int. Cl. A61K 31/397; C07D 413/04; A61P 3/00; A61P 19/00; A61P 29/00; A61P 37/00

(52) U.S. Cl. 514/210.02; 540/355

(57) ABSTRACT

Described herein are substituted 2-(azetidin-2-on-1-yl)alkanoic acids, alkanedioic acids and 2-hydroxyalkyl alkanoic acids, and 2-acyl alkanoic acids, and derivatives thereof, that are capable of modulating activity at the cannabinoid-1 (CB1) and/or cannabinoid-2 (CB2) receptor. Also described herein are methods for treating mammals in need of relief from disease states associated with and responsive to modulation of the CB1 and/or CB2 receptor activity.

### TECHNICAL FIELD

The present invention relates to compounds capable of modulating activity at the cannabinoid-1 (CB1) and/or cannabinoid-2 (CB2) receptor. The present invention also relates to methods for treating mammals in need of relief from disease states associated with and responsive to modulation of the CB1 and/or CB2 receptor activation.

### BACKGROUND

Marijuana (Cannabis sativa L) and its derivatives have been used for centuries for medicinal and recreational purposes. One of the major active ingredients in marijuana, as well as in hashish, is Δ⁹-tetrahydrocannabinol (Δ⁹-THC). In addition, studies on the effects of cannabis (marijuana) have led to the recent discovery of an endogenous system of ligands in humans involved in a number of physiological processes including pain and inflammation. The main naturally occurring ligands for this system, anandamide and 2-arachidonoylglycerol (2-AG), activate a number of cannabinoid receptors, including CB1 and CB2 receptors. Detailed research has revealed that the biological action of Δ⁹-THC and other members of the cannabinoid family of compounds occurs through two related G-protein coupled receptors, the cannabinoid-1 (CB1) receptor, and the cannabinoid-2 (CB2) receptor. The CB1 receptor is primarily found in the central and peripheral nervous systems, mainly the brain and spinal cord, and to a lesser extent in several peripheral organs, including neurons, endocrine glands, leukocytes, spleen, heart and parts of the reproductive, urinary and gastrointestinal tracts. In contrast, the CB2 receptors are expressed primarily by immune cells and tissues (leukocytes, spleen and tonsils), and have been shown to be involved in pain and inflammatory responses.

Cannabinoid receptor “agonists” are compounds that activate the CB1 and CB2 receptors either selectively or non-selectively. Binding to a receptor triggers an event or series of events in the cell that results in the cell's activity, its gene regulation or the signals it sends to neighboring cells. The binding of Δ⁹-THC and other related compounds that activate the CB1 and CB2 receptors non-selectively results in the well-known pain relieving and euphoric benefits associated with the use of marijuana and hashish.

In contrast, receptor “antagonists” and/or “inverse agonists” selectively bind to a receptor that would have been otherwise been available for binding to an endogenous ligand, or some other compound or drug. Antagonists and inverse agonists block the effects of agonists. Such negative modulation of the CB1 and CB2 receptors is important in the treatment of several diseases. In particular, negative modulation of the CB1 receptors may be useful is treating diseases such as obesity, substance abuse disorders, and others. One such modulator, which may be acting as either an inverse agonist or as an antagonist at the CB1 receptor, is rimonabant (5-(4-chlorophenyl)-1-(2,4-dichloro-phenyl)-4-methyl-N-(piperidin-1-yl)-1H-pyrazole-3-carboxamide, SR141716A). Rimonabant has been used in clinical trials for treating of eating disorders, establishing the importance of the CB1 receptor in eating disorders, appetite suppression, obesity treatment, and other addictive behaviors.

Further, negative modulation of the CB2 receptors may be useful is treating diseases such as osteoporosis, gastrointestinal tract diseases, renal ischemia treatment and in the case of inflammatory states. In addition, more particularly regulating substances acting as cannabinoid receptor inverse agonist are effective for chronic and intractable allergies diseases, for which existing therapeutic agents of allergic disease have low effects, and are potentially safe pharmaceutical agents.

Allergies may be recognized as a hypersensitive biological reaction based on an antigen-antibody reaction, which is different from general inflammatory reactions involving the characteristic accumulation of inflammatory response cells, such as monocytes, macrophages and neutrophils. In contrast, it is eosinophils, basophils and mast cells that are largely involved in allergic reactions. Invading antigens (allergens) are incorporated into an antigen presenting cell, such as a macrophage, which causes a cascade through T cells and B cells to produce an antigen-specific IgE antibody, which binds to a mast cell, so that the mast cell is sensitized. Subsequent invasion by the same antigen causes various chemical mediators, such as histamine, eosinophil chemotactic factor and leukotrienes, and cytokines, such as interleukin, to be released from the mast cells. Illustratively, when such chemical mediators act on the bronchi, the smooth muscle of the bronchi constricts to cause mucosal swelling and sputum secretion to narrow its airway, finally causing asthma attack. When such chemical mediators exert its action on skin, inflammation, swelling and itching may occur to cause dermal diseases such as urticaria. When such chemical mediators act on nasal mucus, vascular permeability is increased and exudate draws out of blood which may cause swelling of nasal mucosa leading to nasal occlusion or allergic rhinitis involving sternutation and discharge of a large volume of pituita via nervous irritation. When the reaction occurs in digestive tract, intestinal smooth muscle constricts to abnormally increase intestinal motion (vermiculation), causing digestive allergies such as abdominal pain, vomiting and diarrhea.

Allergic reaction may generally be classified into four types, which may occur separately or in various groups. Type I allergic reaction or immediate type allergic reaction generally occurs within 30 minutes after antigen invasion. Generally, the immediate type allergic reaction disappears in about one hour. Typical diseases of the immediate type allergic reaction include anaphylaxis, allergic rhinitis, pollenosis, urticaria and allergic gastrointestinal diseases. However, several hours to several days later, eosinophils comprising highly toxic chemical mediators may gather around the site of allergic reaction due to eosinophilic chemotactic factors and cytokines that are released from mast cells. The gathered eosinophils release chemical mediators to trigger tissue damages causing a “late phase allergic reaction.” When this reaction occurs in bronchi, the mucoepithelium detaches and antigens more readily invade in the bronchi, leading to prolonged allergic reaction and elevation of the hypersensitivity of airway, making asthma intractable, referred to as late asthmatic response. For example, such late phase response mainly occur after 4 to 8 hours in the case of asthma and mainly occur after 12 to 48 hours in the case of atopic dermatitis.

Type II allergic reactions, also referred to as cytolysis type allergic reaction, occur when complements act on antigen bound IgM or IgG antibody to open holes through the cell membrane to lyse cells. In addition, a reaction occurs wherein macrophages or killer cells act on antibody bound cells and release damaging substances to damage the cells or tissue. Typical diseases of the type II allergic reaction include hemolytic anemia, idiopathic thrombocytopenic purpura, myasthenia gravis and Goodpasture syndrome.

Type III allergic reactions occur when phagocytes cannot process antigen-antibody complexes composed of an antigen and an antibody (IgG antibody) bound together, and the antigen-antibody complexes deposit on tissues. Then, complement, macrophages and neutrophils accumulate on the deposited site to cause inflammation and damage the tissues. Typical diseases of the type III allergic reaction include acute glomerulonephritis induced by hemolytic streptococcus, rheumatoidarthritis, collagen disease, serum sickness, viral hepatitis and allergic alveolitis.

Type IV allergic reactions are different from the type I to III reactions in that no antibody is involved in the reaction. Provided that sensitization with an antigen is established, when the antigen infiltrates again, T cells release cytokines to migrate immune cells such as lymphocytes, neutrophils and macrophages, and destroy the antigen, but at the same time induces inflammation to cause tissue damages. When the infiltrating antigen is a cell, killer T cell damages the cell (antigen). The reaction is generally completed in one to 2 days, and is also referred to as “delayed-type allergic reaction.” Type IV allergic reactions include tuberculin reaction, tuberculosis lesion, post-organ grafting rejections and dermatitis, such as rash against Japanese lacquer (urushi, poison oak, poison ivy, poison sumac) and rash against cosmetics.

Based on the foregoing, there is a need for compounds which are capable of modulating and selectively modulating the CB1 and/or CB2 receptor for cannabinoids and, therefore, may be used in treating the pathologies associated with that receptor.

### SUMMARY OF THE INVENTION

Described herein are compounds capable of binding to cannabinoid-1 receptors (CB1) receptors. In addition, compounds are described herein that modulate the signaling of CB1 receptors. In particular, compounds are described herein that negatively modulate the signaling of CB1 receptors. As described herein, negative modulation of CB1 receptors includes both antagonism and/or inverse agonism of the receptor. In addition, in another aspect, it is appreciated that negative modulation may also include overall down regulation of CB1 receptor expression.

Also, described herein are compounds capable of binding to cannabinoid-2 receptors (CB2) receptors. In addition, compounds are described herein that modulate the signaling of CB2 receptors. In particular, compounds are described herein that negatively modulate the signaling of CB2 receptors. As described herein, negative modulation of CB2 receptors includes both antagonism and/or inverse agonism of the receptor. In addition, in another aspect, it is appreciated that negative modulation may also include overall down regulation of CB2 receptor expression.

It has been found that certain compounds within the general class of 2-(azetidin-2-on-1-yl)alkanoic acids and derivatives thereof elicit activity at CB1 and/or CB2 receptors. Described herein are various substituted 2-(azetidin-2-on-1-yl)alkanoic acids, alkanedioic acids, and 2-hydroxyalkyl alkanoic acids, and 2-acyl alkanoic acids. Also described herein are various derivatives of such compounds, including but not limited to ethers, ketals, esters, amides, carbamates, and ureas of such compounds. For each of the foregoing, pharmaceutical acceptable salts, solvates and hydrates are also described herein. It is to be understood, unless otherwise indicated, that reference to the compounds and compound structures described herein, including pharmaceutically acceptable salts thereof, is intended to be inclusive of the many solvate and hydrate forms. It is appreciated that the compounds and compound structures described herein, including pharmaceutically acceptable salts thereof, include a number of polar functional groups, such as amides, esters, amines, hydroxyl groups, and the like, and that such functional groups among others will readily form solvated and hydrated forms.

Also described herein are pharmaceutical compositions that include therapeutically effective amounts of those substituted 2-(azetidin-2-on-1-yl)alkanoic acids, alkanedioic acids, and 2-hydroxyalkyl alkanoic acids, and 2-acyl alkanoic acids, and derivatives thereof. In addition, methods useful for treating diseases and disease states that are associated with CB1 and/or CB2 regulation or dysfunction, and responsive to antagonism or inverse agonism of CB1 and/or CB2 receptors in mammals are described using those substituted 2-(azetidin-2-on-1-yl)alkanoic acids, alkanedioic acids, and 2-hydroxyalkyl alkanoic acids, and 2-acyl alkanoic acids, and derivatives thereof. In addition, processes for preparing those substituted 2-(azetidin-2-on-1-yl)alkanoic acids, alkanedioic acids, and 2-hydroxyalkyl alkanoic acids, and 2-acyl alkanoic acids, and derivatives thereof are described herein.

In one illustrative embodiment of the methods described herein, one or more compounds of the formula:

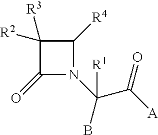

and pharmaceutically acceptable salts thereof, are administered to the patient; wherein

A is —OH or —NH₂; or A is taken together with the attached carbonyl group to form an ester or an amide;

B is a carboxylic acid, or an ester or amide derivative thereof; or B is alkyl, arylalkyl, hydroxyalkyl, alkylthiol, arylhydroxyalkyl, arylalkylthiol, aminoalkyl, or acyl, each of which is optionally substituted, or a derivative thereof, including ethers, esters, amides, carbonates, carbamates, ureas, ketals, and the like;

R¹ is hydrogen or C₁-C₆ alkyl;

R² is hydrogen, alkyl, alkenyl, alkynyl, alkoxy, alkylthio, halo, haloalkyl, cyano, formyl, alkylcarbonyl, or a substituent selected from the group consisting of —CO₂R⁸, —CONR⁸R^{8′}, and —NR⁸(COR⁹); where R⁸ and R^{8′} are each independently selected from hydrogen, alkyl, cycloalkyl, optionally substituted aryl, or optionally substituted arylalkyl; or R⁸ and R^{8′} are taken together with the attached nitrogen atom to form a heterocyclyl group; and where R⁹ is selected from hydrogen, alkyl, cycloalkyl, alkoxyalkyl, optionally substituted aryl, optionally substituted arylalkyl, optionally substituted heteroaryl, optionally substituted heteroarylalkyl, and R⁸R^{8′}N—(C₁-C₄ alkyl);

R³ is an amino, amido, acylamido, or ureido group, which is optionally substituted; or R³ is a nitrogen-containing heterocyclyl group attached at a nitrogen atom;

R⁴ is alkyl, alkenyl, alkynyl, cycloalkyl, cycloalkenyl, alkylcarbonyl, optionally substituted aryl, optionally substituted arylalkyl, optionally substituted arylhaloalkyl, optionally substituted arylalkoxyalkyl, optionally substituted arylalkenyl, optionally substituted arylhaloalkenyl, or optionally substituted arylalkynyl;

R⁸ and R^{8′} are each independently selected from hydrogen, alkyl, including C₁-C₆ alkyl, cycloalkyl, including C₃-C₈ cycloalkyl, optionally substituted aryl, or optionally substituted arylalkyl, including aryl(C₁-C₄ alkyl); or R⁸ and R^{8′} are taken together with the attached nitrogen atom to form an heterocycle, such as optionally substituted pyrrolidinyl, piperidinyl, morpholinyl, piperazinyl, and homopiperazinyl; and

R⁹ is selected from hydrogen, alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including aryl(C₁-C₄ alkyl), optionally substituted heteroaryl, optionally substituted heteroarylalkyl, including heteroaryl(C₁-C₄ alkyl), and R⁸R^{8′}N—(C₁-C₄ alkyl).

In another illustrative embodiment of the methods described herein, one or more compounds of formula (I):

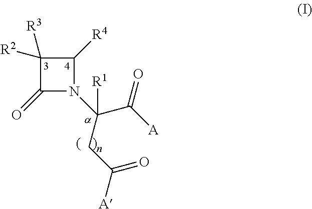

and pharmaceutically acceptable salts thereof, are administered to the patient; wherein

A and A′ are each independently selected from —OH and —NH₂; or A and/or A′ are taken together with the attached carbonyl group to form an ester or an amide;

n is an integer selected from 0 to about 3;

R¹ is hydrogen or C₁-C₆ alkyl;

R² is hydrogen, alkyl, alkenyl, alkynyl, alkoxy, alkylthio, halo, haloalkyl, cyano, formyl, alkylcarbonyl, or a substituent selected from the group consisting of —CO₂R⁸, —CONR⁸R^{8′}, and —NR⁸(COR⁹); where R⁸ and R^{8′} are each independently selected from hydrogen, alkyl, cycloalkyl, optionally substituted aryl, or optionally substituted arylalkyl; or R⁸ and R^{8′} are taken together with the attached nitrogen atom to form an heterocycle; and where R⁹ is selected from hydrogen, alkyl, cycloalkyl, alkoxyalkyl, optionally substituted aryl, optionally substituted arylalkyl, optionally substituted heteroaryl, optionally substituted heteroarylalkyl, and R⁸R^{8′}N—(C₁-C₄ alkyl);

R³ is an amino, amido, acylamido, or ureido group, which is optionally substituted; or R³ is a nitrogen-containing heterocyclyl group attached at a nitrogen atom; or R³ is an optionally substituted aryl group; and

R⁴ is alkyl, alkenyl, alkynyl, cycloalkyl, cycloalkenyl, alkylcarbonyl, optionally substituted aryl, optionally substituted arylalkyl, optionally substituted arylhaloalkyl, optionally substituted arylalkoxyalkyl, optionally substituted arylalkenyl, optionally substituted arylhaloalkenyl, or optionally substituted arylalkynyl.

In another illustrative embodiment of the methods described herein, one or more compounds of formula (II):

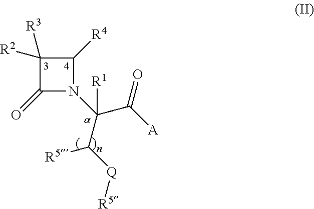

and pharmaceutically acceptable salts thereof, are administered to the patient; wherein

A is —OH or —NH₂; or A is taken together with the attached carbonyl group to form an ester or an amide;

Q is oxygen; or Q is sulfur or disulfide, or an oxidized derivative thereof;

n is an integer from 1 to 3;

R¹, R², R³, and R⁴ are as defined in formula I;

R^{5″} is selected from hydrogen, alkyl, cycloalkyl, alkoxyalkyl, optionally substituted arylalkyl, optionally substituted heterocyclyl or optionally substituted heterocyclylalkyl, and optionally substituted aminoalkyl; and

R^{5′″} is selected from hydrogen, alkyl, and optionally substituted arylalkyl.

In another illustrative embodiment, compounds of formula (III) are described:

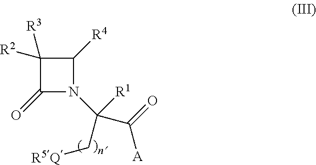

and pharmaceutically acceptable salts thereof, are administered to the patient; wherein

n is an integer in the range from about 1 to about 5, and is illustratively 1, 2, or 3;

A is —OH or —NH₂; or A is taken together with the attached carbonyl group to form an ester or an amide;

Q′ is oxygen or sulfur;

R¹, R², R³, and R⁴ are as defined in formula I;

R^{5′} is selected from the group consisting of C₁-C₆ alkyl, C₃-C₈ cycloalkyl, (C₁-C₄ alkoxy)-(C₁-C₄ alkyl), optionally-substituted aryl(C₁-C₄ alkyl), Y′—(C₁-C₄ alkyl), where Y′- is a heterocycle, and R^{6′}R^{7′}N—(C₂-C₄ alkyl); where Y′ is selected from the group consisting of tetrahydrofuryl, morpholinyl, pyrrolidinyl, piperidinyl, piperazinyl, homopiperazinyl, or quinuclidinyl; where said morpholinyl, pyrrolidinyl, piperidinyl, piperazinyl, homopiperazinyl, or quinuclidinyl is optionally N-substituted with C₁-C₄ alkyl or optionally-substituted aryl(C₁-C₄ alkyl);

R^{6′} is hydrogen or alkyl, including C₁-C₆ alkyl, and R^{7′} is alkyl, including C₁-C₆ alkyl, cycloalkyl, including C₃-C₈ cycloalkyl, optionally substituted aryl, or optionally substituted arylalkyl, including aryl(C₁-C₄ alkyl); or R^{6′} and R^{7′} are taken together with the attached nitrogen atom to form an heterocycle, such as pyrrolidinyl, piperidinyl, morpholinyl, piperazinyl, and homopiperazinyl; where said piperazinyl or homopiperazinyl is optionally N-substituted with R^{13′}; and

R^{13′} is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

In another illustrative embodiment, compounds of formula (IV) are described:

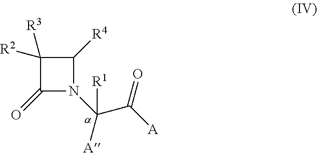

and pharmaceutically acceptable salts thereof, are administered to the patient; wherein

A is —OH or —NH₂; or A is taken together with the attached carbonyl group to form an ester or an amide;

A″ is hydrogen, halo, alkyl, cycloalkyl, aryl, heteroaryl, arylalkyl, heteroarylalkyl, aminoalkyl or a derivative thereof, alkylcarbonyl, cycloalkylcarbonyl, arylcarbonyl, heteroarylcarbonyl, arylalkylcarbonyl, or heteroarylalkylcarbonyl, each of which may be optionally substituted; and where the carbonyl of each is optionally an alkylene, arylalkylene, or heteroarylalkylene ketal; and

R¹, R², R³, and R⁴ are as defined in formula (I).

In another illustrative embodiment of the methods described herein, one or more compounds of formula (V):

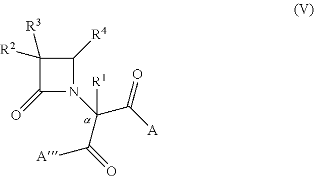

and pharmaceutically acceptable salts thereof, are administered to the patient; wherein A is a —CO₂H, or an ester or an amide derivative thereof; A′″ is alkyl, cycloalkyl, aryl, heteroaryl, arylalkyl, or heteroarylalkyl, each of which may be optionally substituted; and R¹, R², R³, and R⁴ are as defined in formula (I).

In each of the foregoing embodiments, various selections may be made for each of the substituents defined therein. Illustratively, the group R³ may be selected from the following group of formulae:

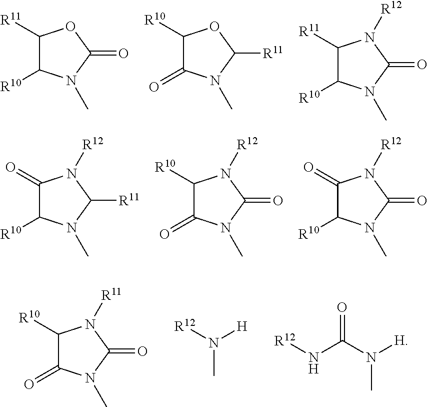

wherein R¹⁰ and R¹¹ are each independently selected from hydrogen, optionally substituted alkyl, including C₁-C₆ alkyl, optionally substituted cycloalkyl, including C₃-C₈ cycloalkyl, alkoxyalkyl, including C₁-C₄ alkoxycarbonyl, alkylcarbonyloxy, including C₁-C₅ alkylcarbonyloxy, optionally substituted aryl, optionally substituted arylalkyl, including aryl(C₁-C₄ alkyl), optionally substituted arylalkyloxy, including aryl(C₁-C₄ alkyloxy), optionally substituted arylalkylcarbonyloxy, including aryl(C₁-C₄ alkylcarbonyloxy), diphenylmethoxy, and triphenylmethoxy; and

R¹² is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

In another illustrative selection, R³ is of the formulae:

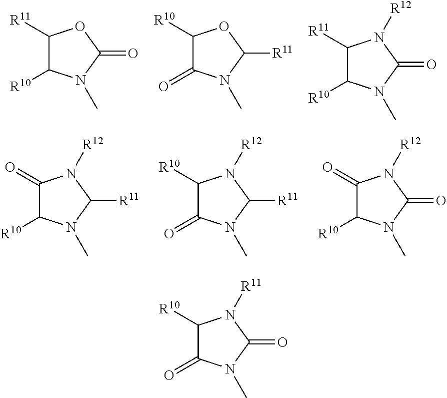

of the formulae:

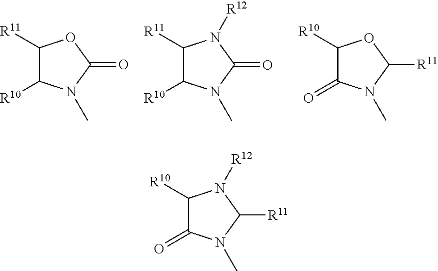

or of the formula:

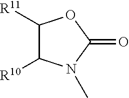

wherein R¹⁰, R¹¹, and R¹² are as defined herein.

It is appreciated that other selections for various substituents may be made, as described herein, and that each selection or collection of selections can be made in each of formulae (I)-(V).

In another embodiment, pharmaceutical compositions are described herein, where the pharmaceutical compositions include one or more of the compounds described herein, including but not limited to the compounds of formulae (I)-(V), substituted 2-(azetidin-2-on-1-yl)alkanedioic acids, substituted 2-(azetidin-2-on-1-yl)hydroxyalkylalkanoic acids, substituted 2-(azetidin-2-on-1-yl)hydroxyalkylalkanoic acids, and/or substituted 2-(azetidin-2-on-1-yl)alkylalkanoic acids, including analogs and derivatives thereof described herein, and combinations thereof. The substituted 2-(azetidin-2-on-1-yl)alkanedioic acids, substituted 2-(azetidin-2-on-1-yl)hydroxyalkylalkanoic acids, substituted 2-(azetidin-2-on-1-yl)alkylalkanoic acids, and derivatives thereof include compounds of formulae (I)-(V). The pharmaceutical compositions described herein also include one or more pharmaceutically acceptable carriers, diluents, and/or excipients. In one illustrative aspect, pharmaceutical compositions are described that exhibit oral activity and/or oral bioavailability. In another illustrative aspect, pharmaceutical compositions are described that allow the substituted 2-(azetidin-2-on-1-yl)alkanedioic acids, substituted 2-(azetidin-2-on-1-yl)hydroxyalkylalkanoic acids, substituted 2-(azetidin-2-on-1-yl)alkylalkanoic acids, and analogs and derivatives thereof to cross the blood brain barrier.

In another embodiment, methods for treating disease states, disorders, conditions, or symptoms that are responsive to modulation, and in particular negative modulation of CB1 receptors are described herein. Such methods include the step of administering a therapeutically effective amount of one or more compounds of formulae (I)-(V), or a pharmaceutical composition thereof, to an animal, such as a human or other invertebrate, in need of such treatment. Illustratively, those disease states and disorders include obesity, eating disorders (including binge eating disorder, anorexia, and bulimia), weight loss or control (e.g., reduction in calorie or food intake, and/or appetite suppression), addictive behaviors, substance abuse disorders and drug addiction (including alcohol, cocaine, heroin, marijuana, and nicotine addictions), allergies, dementia (including memory loss, Parkinson's disease, Alzheimer's disease, dementia of aging, vascular dementia, mild cognitive impairment, age-related cognitive decline, and mild neurocognitive disorder), bone disorders (including osteoclast dysregulation, osteoblast dysregulation, osteoporosis, osteopenia, and Paget's disease), and the like.

In another embodiment, methods for treating disease states, disorders, conditions, or symptoms that are responsive to modulation, and in particular negative modulation of CB2 receptors are described herein. Such methods include the step of administering a therapeutically effective amount of one or more compounds of formulae (I)-(V), or a pharmaceutical composition thereof, to an animal, such as a human or other invertebrate, in need of such treatment. Illustratively, those disease states and disorders include those responsive to or mediated by CB2 inverse agonists or antagonists, such as by the inhibition of osteoclasts (for example, the inhibition of the survival, formation, and/or activity of osteoclasts), and/or in the inhibition of bone resorption. Such methods are useful in the treatment of bone disorders, such as conditions mediated by osteoclasts (e.g., increased osteoclast activity) characterized by for example increased, bone resorption, such as osteoporosis, such as osteoporosis associated with or not associated with inflammation; osteoporosis associated with genetic predisposition, sex hormone deficiency, or ageing; cancer associated bone disease; and Paget's disease of bone.

In another embodiment, the CB2 receptor modulating compounds described herein may be used in pharmaceutical compositions methods for treating a variety of diseases including but not limited to immune disorder, inflammation, osteoporosis and renal ischemia.

In one variation, the compounds described herein modulate CB1 and/or CB2 receptors by a mechanism including antagonism of the receptor. Such antagonism may be of an endogenous agonist, or a co-administered exogenous agonist. In another variation, the compounds described herein modulate CB1 and/or CB2 receptors by a mechanism including inverse agonism of the receptor. Such inverse agonism may be of a constitutively expressed CB1 and/or CB2 receptor.

In one embodiment of the invention, modulators of CB1 and/or CB2 receptor activation are described herein. Those modulators are of the general formulae (I)-(V), including selected enantiomers, diastereomers, and mixtures thereof, and pharmaceutically acceptable salts, hydrates, solvates, and polymorphs thereof. The general chemical terms used in the formulae described herein have their usual ordinary meanings. Illustratively, the term “alkyl” refers to a straight-chain or optionally branched, saturated hydrocarbon; “cycloalkyl” refers to a straight-chain or optionally branched, saturated hydrocarbon, at least a portion of which forms a ring; “alkenyl” refers to a straight-chain or optionally branched, hydrocarbon that includes at least one double bond; “aryl” refers to an aromatic ring or heteroaromatic ring; and “heterocycle” refers to a non-aromatic cyclic structure possessing one or more heteroatoms, such as nitrogen, oxygen, sulfur.

Further, the term “acyl” refers to both “alkanoyl” and “aroyl” and includes alkyl, alkenyl, aryl, heteroaryl, and the like attached through a carbonyl group.

The term “optionally substituted” refers to the replacement of one or more, illustratively from one to about three, hydrogen atoms with one or more substitutents. Substituents include but are not limited to such groups as C₁-C₄ alkyl, C₁-C₄ alkoxy, C₁-C₄ alkylthio, hydroxy, nitro, halo, carboxy, cyano, C₁-C₄ haloalkyl, C₁-C₄ haloalkoxy, amino, carbamoyl, carboxamido, amino, alkylamino, dialkylamino, alkylalkylamino, C₁-C₄ alkylsulfonylamino, and the like.

The term “protected amino” refers to amine protected by a protecting group that may be used to protect the nitrogen, such as the nitrogen in the β-lactam ring, during preparation or subsequent reactions. Examples of such groups are benzyl, 4-methoxybenzyl, 4-methoxyphenyl, trialkylsilyl, for example trimethylsilyl, and the like. The term “protected carboxy” refers to the carboxy group protected or blocked by a conventional protecting group commonly used for the temporary blocking of the acidic carboxy. Examples of such groups include lower alkyl, for example tert-butyl, halo-substituted lower alkyl, for example 2-iodoethyl and 2,2,2-trichloroethyl, benzyl and substituted benzyl, for example 4-methoxybenzyl and 4-nitrobenzyl, diphenylmethyl, alkenyl, for example allyl, trialkylsilyl, for example trimethylsilyl and tert-butyldiethylsilyl and like carboxy-protecting groups. Illustrative protecting groups are described in Greene & Wuts, “Protective Groups ion Organic Synthesis,” 2d edition, John Wiley & Sons, Inc. New York (1991), the disclosure of which in its entirety is incorporated herein by reference.

### BRIEF DESCRIPTION OF THE DRAWINGS

FIG. 1 shows the K_{d} for CP55940 displacement of ³H-CP55940 on CB1 receptors as a function of Log M of CP55940; IC₅₀=2.0 nM; K_{d}=1.0 nM.

FIG. 2 shows the competitive Binding of Example 70C by displacement of ³H-CP55940 on CB1 receptors as a function of Log M of Example 70C IC₅₀=1.35 μM; Kᵢ=0.676 μM.

FIG. 3a shows a positive control demonstrating agonism of CB1 by WIN 55212-2.

FIG. 3b shows a positive control demonstrating antagonism of WIN 55212-2 by AM251 at CB1 receptors.

FIG. 3c shows antagonism of WIN 55212-2 by Example 37S at CB1 receptors.

FIG. 3d shows antagonism of WIN 55212-2 by Example 70C at CB1 receptors.

FIG. 4 shows a positive control demonstrating agonism of CB1 by WIN 55212-2.

FIG. 5a shows antagonism by Example 37S at CB1 receptors.

FIG. 5b shows antagonism of 3 μM WIN 55212-2 by Example 37S at CB1 receptors.

FIG. 6a shows antagonism by Example 37U at CB1 receptors.

FIG. 6b shows antagonism of 3 μM WIN 55212-2 by Example 37U at CB1 receptors.

FIG. 7a shows antagonism by Example 70C at CB1 receptors.

FIG. 7b shows antagonism of 3 μM WIN 55212-2 by Example 70C at CB1 receptors.

FIG. 8 shows the binding of Example 33G against pre-bound CP55940 on CHO cells expressing hCB2 receptors; IC50=273 nM, Kᵢ=182 nM.

FIG. 9a shows a positive control of agonism of CB2 receptors by CP-55940 (▪) and negative control by single concentration of Example 38J (▴).

FIG. 9b shows the functional activity of AM630 at CB2 in the presence of 3 μM CP-55940.

FIG. 9c shows the functional activity of Example 62B at CB2 in the presence of 3 μM CP-55940.

FIG. 9d shows the functional activity of Example 31A at CB2 in the presence of 3 μM CP-55940.

FIG. 9e shows the functional activity of Example 33Gat CB2 in the presence of 3 μM CP-55940.

FIG. 9f shows the functional activity of Example 38ARat CB2 in the presence of 3 μM CP-55940.

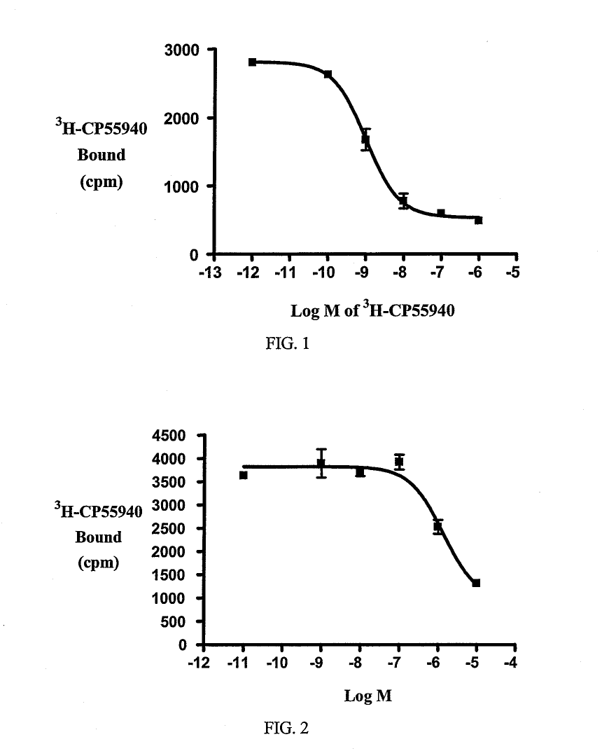

### DETAILED DESCRIPTION

In one embodiment, methods for treating disease states, disorders, conditions, or symptoms that are responsive to modulation, and in particular negative modulation of CB1 receptors are described herein. Such methods include the step of administering a therapeutically effective amount of one or more compounds of formula (I), or a pharmaceutical composition thereof, to an animal in need of such treatment. Illustratively, those disease states and disorders include obesity, eating disorders (including binge eating disorder, anorexia, and bulimia), weight loss or control (e.g., reduction in calorie or food intake, and/or appetite suppression), allergies, bone disorders (including osteoporosis), and the like.

In another embodiment, those disease states and disorders include substance abuse which includes alcoholism (e.g., alcohol abuse, addiction and/or dependence including treatment for abstinence, craving reduction and relapse prevention of alcohol intake), tobacco abuse (e.g., smoking addiction, cessation and/or dependence including treatment for craving reduction and relapse prevention of tobacco smoking), drug addiction (including, cocaine, heroin, and marijuana addictions), and also suppression of reward-related behaviors (e.g., conditioned place avoidance, such as suppression of cocaine- and morphine-induced conditioned place preference).

In another embodiment, those disease states and disorders include dementia (including memory loss, Alzheimer's disease, dementia of aging, vascular dementia, mild cognitive impairment, age-related cognitive decline, and mild neurocognitive disorder), memory deficits, cognitive disorders, movement disorders, and Parkinson's disease.

In another embodiment, those disease states and disorders include behavioral addictions, and addictive disorders.

In another embodiment, those disease states and disorders include attention deficit disorder (including ADHD), bipolar disorders, and impulsivity.

In another embodiment, those disease states and disorders include epilepsy, seizure disorders, schizophrenia, atypical depression, depression, and stress.

In another embodiment, those disease states and disorders include neuropathy, neuro-inflammatory disorders including multiple sclerosis and Guillain-Baree syndrome and the inflammatory sequelae of viral encephalitis,

In another embodiment, those disease states and disorders include sexual dysfunction in males (including erectile difficulty, premature ejaculation, and the like),

In another embodiment, those disease states and disorders include constipation and chronic intestinal pseudo-obstruction, and gastrointestinal disorders (e.g., dysfunction of gastrointestinal motility or intestinal propulsion).

In another embodiment, those disease states and disorders include injuries resulting from cerebral vascular accidents and head trauma, migraine, and inflammation.

In another embodiment, those disease states and disorders include anxiety disorders, asthma, cirrhosis of the liver, psychosis, psychoses, and type II diabetes.

In one aspect, the method includes the use of a cannabinoid receptor inverse agonist or a cannabinoid receptor neutral antagonist, as described herein, for the manufacture of a medicament for use in the treatment of a condition mediated by osteoclasts (e.g., increased activity) characterized by (e.g., increased) bone resorption, as described herein.

In another aspect, the method includes the use of a cannabinoid receptor inverse agonist or a cannabinoid receptor neutral antagonist, as described herein, for the manufacture of medicament for use in the treatment of condition mediated by osteoclasts herein.

In another aspect, the method includes the use of a cannabinoid receptor inverse agonist or a cannabinoid receptor neutral antagonist, as described herein, for the manufacture of a medicament for use in the treatment of condition characterized by (e.g., increased) bone resorption, as described herein.

In another aspect, the method includes the use of a cannabinoid receptor inverse agonist or a cannabinoid receptor neutral antagonist, as described herein, for the manufacture of a medicament for use in the treatment of osteoporosis (e.g., osteoporosis not associated with inflammation; e.g., osteoporosis associated with a genetic predisposition, sex hormone deficiency, or ageing).

In another aspect, the methods described herein include the step of administering a cannabinoid receptor inverse agonist or a cannabinoid receptor neutral antagonist, as described herein, to a patient for use in a method of treatment of the human or animal body by therapy.

In another aspect, the methods described herein include the step of administering a cannabinoid receptor inverse agonist or a cannabinoid receptor neutral antagonist, as described herein, to a patient for use in a method of treatment of a condition mediated by osteoclasts (e.g., increased activity) and/or characterised by (e.g., increased) bone resorption, as described herein, of the human or animal body by therapy.

In another aspect, the methods described herein include the step of administering a cannabinoid receptor neutral antagonist, as described herein, to a patient for use in a method of treatment of a condition mediated by osteoclasts (e.g., increased osteoclast activity), as described herein, of the human or animal body by therapy.

In another aspect, the methods described herein include the step of administering a cannabinoid receptor neutral antagonist, as described herein, to a patient for use in a method of treatment of a condition characterised by (e.g., increased) bone resorption, as described herein, of the human or animal body by therapy.

In another aspect, the methods described herein include the step of administering a cannabinoid receptor neutral antagonist, as described herein, to a patient for use in a method of treatment of osteoporosis (e.g., osteoporosis not associated with inflammation; e.g., osteoporosis associated with a genetic predisposition, sex hormone deficiency, or ageing).

In another aspect, a method of treating a bone disorder is described herein, the method comprising the step of administering to a patient in need of treatment thereof a effective amount of a cannabinoid receptor inverse agonist or a cannabinoid receptor neutral antagonist, as described herein, preferably in the form of a pharmaceutical composition.

In another aspect, a method for the treatment of a condition mediated by (e.g., increased osteoclast activity) and/or characterised by (e.g., increased) bone resorption is described herein, the method comprising the step of administering to a subject suffering from said condition a therapeutically-effective amount of a cannabinoid receptor inverse agonist or a cannabinoid receptor neutral antagonist, as described herein, preferably in the form of a pharmaceutical composition.

In another aspect, a method for the treatment of a condition mediated by-osteoclasts increased osteoclast activity is described herein, the method comprising the step of administering to a subject suffering from said condition a therapeutically-effective amount of a cannabinoid receptor inverse agonist or a cannabinoid receptor neutral antagonist, as described herein, preferably in the form of a pharmaceutical composition.

In another aspect, a method for the treatment of a condition characterized by for example increased bone resorption is described herein, the method comprising the step of administering to a subject suffering from said condition a therapeutically-effective amount of a cannabinoid receptor inverse agonist or a cannabinoid receptor neutral antagonist, as described herein, preferably in the form of a pharmaceutical composition.

In another aspect, a method for the treatment of osteoporosis (e.g., osteoporosis not associated with inflammation; e.g., osteoporosis associated with a genetic predisposition, sex hormone deficiency, or ageing), cancer associated bone disease, or Paget's disease of bone is described herein, the method comprising the step of administering to subject suffering from said condition therapeutically-effective amount of a cannabinoid receptor inverse agonist or a cannabinoid receptor neutral antagonist, as described herein, preferably in the form of pharmaceutical composition.

In one embodiment, the bone disorder is osteoporosis (e.g., osteoporosis not associated with inflammation; e.g., osteoporosis associated with a genetic predisposition, sex hormone deficiency, or ageing), cancer associated bone disease, and Paget's disease of bone.

The cannabinoid receptor inverse agonists and cannabinoid receptor neutral antagonists, as described herein, are useful in the treatment of bone disorders, for example, conditions mediated by osteoclasts (e.g., by increased osteoclast activity) (as “osteoclast inhibitors”), and/or conditions characterized by increased bone resorption (as “bone resorption inhibitors”).

In one embodiment, the bone disorder is characterized by increased osteoclast activity. In one embodiment, the bone disorder is characterized by increased bone resorption. In one embodiment, the bone disorder is associated with genetic predisposition, sex hormone deficiency, or ageing. For example, in one embodiment, the bone disorder is characterized by increased bone resorption, and is associated with a genetic predisposition, sex hormone deficiency, or ageing.

In one embodiment, the bone disorder is not associated with inflammation. For example, in one embodiment, the bone disorder is characterized by increased bone resorption, and is not associated with inflammation. For example, in one embodiment, the bone disorder is characterized by increased bone resorption; and is associated with genetic predisposition, sex hormone deficiency, or ageing; and is not associated with inflammation. For example, in one embodiment, the bone disorder is not associated with rheumatoid arthritis, ankylosing spondylitis, or inflammatory bowel disease. For example, in one embodiment, the bone disorder is characterized by increased bone resorption, and is not associated with rheumatoid arthritis, ankylosing spondylitis, or inflammatory bowel disease. For example, in one embodiment, the bone disorder is characterized by increased bone resorption; and is associated with a genetic predisposition, sex hormone deficiency, or ageing; and is not associated with rheumatoid arthritis, spondylitis, or inflammatory bowel disease.

Examples of such bone disorders include, but are not limited to, the following diseases of the skeleton, including but not limited to, pathologically low bone mineral density, such as osteoporosis (including, e.g., steroid induced osteoporosis) (e.g., osteoporosis not associated with inflammation); osteoarthritis; Paget's disease of bone (osteitis deformans); caused by conditions associated with increased bone resorption, including, but not limited to vitamin D intoxication, primary or tertiary hyperparathyroidism, immobilization, and sarcoidosis; neoplasia of bones, both as a primary tumor and as metastases, including but not limited to, osteosarcoma and osteoma (Zheng et al., J. Cell Biochem. 70:121 (1998)) and cancer associated bone disease (e.g., of malignancy, bone metastases, osteolytic bone metastases, multiple myeloma, breast carcinoma).

In another embodiment, the invention illustratively includes uses of the compounds described herein for the manufacture of medicaments for treating the diseases and disorders described herein as responsive to CB1 modulation. It is to be understood that the compounds described herein may also be combined with other biologically active agents, medicaments, pharmaceuticals, and the like, including nicotine receptor partial agonists, opioid antagonists (such as naltrexone and nalmefene), dopaminergic agents (such as apomorphine), attention deficit disorder (ADD) and attention deficit hyperactivity disorder (ADHD) agents, such as methylphenidate hydrochloride (RITALIN and CONCERTA), atomoxetine (STRATTERA), and amphetamines (ADDERALL), anti-obesity agents, and the like. It is appreciated that such combinations are also useful in treating the diseases and disorders described herein as responsive to CB1 modulation.

In another embodiment, methods for treating disease states, disorders, conditions, or symptoms that are responsive to modulation, and in particular negative modulation of CB2 receptors are described herein. Such methods include the step of administering a therapeutically effective amount of one or more compounds of formula (I), or a pharmaceutical composition thereof, to an animal in need of such treatment. Illustratively, those disease states and disorders include, but are not limited to, osteoporosis, asthma, allergies and allergic reactions.

In another embodiment, cannabinoid receptor-regulating substances are effective as therapeutic agents of allergic disease such as asthma and atopic dermatitis. In one aspect, the compounds described herein may be capable of regulating and selectively acting on peripheral cell type cannabinoid receptors, such as by inverse agonism. Such compounds may be effective for chronic and intractable allergies diseases, especially for those disease states for which existing therapeutic agents of allergic disease have limited benefits.

In one aspect, symptoms of atopic dermatitis include hypersensitivity and dryness of skin. Characteristic exanthema of atopic dermatitis (erythematosus, papule, incrustation, lichen lesion, prurigo, etc.) progress in chronic and recurrent course. Further, the symptoms induce complications such as Kaposi's sarcoma varicelliform eruption, viral infections (infection with herpes simplex virus and the like), impetigo and infectious molluscum (cataract, retinodialysis, and others).

“Asthma” is characterized by its reversible airstream restriction (airway occlusion) and airway hypersensitivity. However, airways suffering from asthma may involve the occurrence of chronic inflammation characterized by detachment of airway epithelium, fibrosis of airway just below the basement membrane (hypertrophy of the basement membrane) and eosinophil accumulation. Currently, asthma is therefore recognized as a chronic inflammatory disease. Many inflammatory cells such as eosinophils, T cells and mast cells are suggested to be involved in airway inflammation. It is considered that the involvement of mast cells, the involvement of eosinophils, and the involvement of eosinophils and CD4-positive helper T cells are important for immediate type response, late phase response and delayed type response, respectively.

In another embodiment, methods for treating allergic diseases are described. Such allergic diseases include but are not limited to anaphylaxis, digestive tract allergy, allergic gastritis, allergic dermatitis, dermatitis such as rash against Japanese lacquer (urushi) and rash against cosmetics, urticaria, atopic dermatitis, asthma, allergic asthma, atopic asthma, allergic bronchial pulmonary aspergillosis, pollenosis, allergic rhinitis, allergic conjunctivitis, allergic sarcoma angitis, chemical allergy, serum disease, post-organ transplantation rejection, tuberculosis lesion and post-organ transplantation rejection.

In another embodiment, methods for treating allergic diseases are described. Such allergic diseases include but are not limited to allergic asthma, atopic dermatitis, allergic rhinitis and allergic conjunctivitis. The methods include the step of administering one or more compounds described herein in an amount effective to provide relief from the allergic disease. The effective amount is dependent upon the animal or patient being treated, and can be readily determined by routine experimentation using the assays and methods described herein. In one aspect, the allergic disease is responsive to the antagonism or inverse agonism of CB2 receptors. In another aspect, the compounds described herein are selective for CB2 receptors, as compared to other receptors, including but not limited to CB1 receptors.

In another embodiment, methods for treating non-immediate-type allergic diseases are described. Such non-immediate-type allergic diseases include but are not limited to allergic dermatitis, atopic dermatitis, allergic asthma and atopic asthma, hemolytic anemia, thrombocytopenic purpura, myasthenia gravis, Goodpasture syndrome, acute glomerulonephritis induced by hemolytic streptococcus, rheumatoid arthritis and connective tissue diseases, serum disease, viral hepatitis, allergic alveolitis, tuberculin reaction, tuberculosis lesion, post-organ transplantation rejections, dermatitis such as urushi, late-type asthma, anaphylaxis, allergic rhinitis, allergic conjunctivitis, pollenosis, urticaria, allergic gastroenteritis and itch that involve late phase and/or delayed-type allergic reactions. In one aspect, the non-immediate-type allergic diseases include allergic dermatitis, atopic dermatitis, allergic asthma, and atopic asthma.

Localization of CB2 receptors on cells of the immune system has led to the suggestion that cannabinoid receptor ligands may be of value as immunosuppressive and anti-inflammatory agents. In one embodiment, the cannabinoid receptor inverse agonist or a cannabinoid receptor neutral antagonist is selective for CB2. Studies in animal models have shown that both CB2 selective agonists (see, e.g., Hanus et 1999) and CB1 selective agonists (see, e.g., et 2002; Smith et 2001) have anti-inflammatory effects. These anti-inflammatory effects are blocked by CB2 selective inverse agonists (Hanus et al, 1999; Conti et al., 2002) and by CB1 selective inverse agonists (Clayton et 2002) respectively. Accordingly, in another embodiment, the cannabinoid receptor inverse agonist or a cannabinoid receptor neutral antagonist exhibits activity at both CB1 and CB2.

In another embodiment, CB2 antagonists can be used for gastrointestinal tract diseases and disorders. CB2 agonists have been reported to inhibit dedecation in mice (Hanes, et al. Proc. Natl. Acad. Sci. U.S.A. 96:14228-12335 (1999), describing HU-308, a specific agonist for peripheral CB2 cannabinoid receptors; CB2 antagonists increase nerve stimulation-elicited relaxation of the rat fundus (Storr, M. et al., Effect of cannabinoids on neural transmission in rat gastric funds. Can J. Pharmacol. 80:67-76 (2002)). Activation of CB2 receptors represents a novel mechanism for the re-establishment of normal gastrointestinal transit after an inflammatory stimulus.

In another embodiment, CB2 receptor antagonists may be useful for the treatment of myocardial infarctions (Hiley, et al, Cannabinoid pharmacology in the cardiovascular system: potential protective mechanisms through lipid signalling, Biol. Reviews 79:187-205 (2004)).

In another embodiment, CB2 receptor antagonists may be useful for the treatment of immunologically-mediated inflammatory diseases such as rheumatoid arthritis, systemic lupus erythematosus, psoriasis, multiple schlerosis, diabetis and thyroiditis. In another embodiment, the compounds described herein may be administered to modulate bone formation/resorption and are therefore useful in the treatment of conditions including but not limited to ankylosing spondylitis, gout, arthritis associated with gout, osteoarthritis, and osteoporosis.

In another embodiment, non selective compounds that are both CB1 and CB2 antagonists may be useful for the treatment of diseases selected from the group consisting of obesity, schizophrenia, epilepsy or cognitive disorders such as Alzheimer's, bone disorders. bulimia, obesity associated with type II diabetes (non-insulin dependant diabetes) or drug, alcohol or nicotine abuse or dependency (WO 2006/054057).

In another embodiment, the invention illustratively includes uses of the compounds described herein for the manufacture of medicaments for treating the diseases and disorders described herein as responsive to CB2 modulation. It is to be understood that the compounds described herein may also be combined with other biologically active agents, medicaments, pharmaceuticals, and the like. It is appreciated that such combinations are also useful in treating the diseases and disorders described herein as responsive to CB1 modulation.

The compounds described herein may also be administered in combination with other pharmaceutical agents. Illustrative other pharmaceutical agents include nicotine receptor partial agonists, dopaminergic agents, such as apomorphine, anti-obesity agents, such as apo-B/MTP inhibitors, 11β-hydroxy steroid dehydrogenase-1 (11β-HSD type 1) inhibitors, peptide YY3-36 and analogs thereof, MCR-4 agonists, CCK-A agonists, monoamine reuptake inhibitors, sympathomimetic agents, β3 adrenergic receptor agonists, dopamine receptor agonists, melanocyte-stimulating hormone receptor analogs, 5-HT2c receptor agonists, melanin concentrating hormone receptor antagonists, leptin, leptin analogs, leptin receptor agonists, galanin receptor antagonists, lipase inhibitors, bombesin receptor agonists, neuropeptide-Y receptor antagonists, such as NPY-5 receptor antagonists, thyromimetic agents, dehydroepiandrosterone and analogs thereof, glucocorticoid receptor antagonists, orexin receptor antagonists, glucagon-like peptide-1 receptor agonists, ciliary neurotrophic factors, human agouti-related protein antagonists, ghrelin receptor antagonists, histamine 3 receptor antagonists and inverse agonists, and neuromedin U receptor agonists, and the like.

Such combination therapies may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order. Yet another aspect of the present invention includes a pharmaceutical kit for use by a consumer to treat diseases, conditions or disorders modulated by cannabinoid receptor antagonists in an animal. The kit comprises a) a suitable dosage form comprising a compound of the present invention; and b) instructions describing a method of using the dosage form to treat diseases, conditions or disorders that are modulated by cannabinoid receptor (in particular, the CB1 receptor) antagonists. Another embodiment includes a pharmaceutical kit comprising: a) a first dosage form comprising (i) a compound of the present invention and (ii) a pharmaceutically acceptable carrier, excipient or diluent; b) a second dosage form comprising (i) an additional pharmaceutical agent described herein, and (ii) a pharmaceutically acceptable carrier, excipient or diluent; and c) a container.

It is appreciated that when using a combination of the present invention, the CB1 receptor modulator and the antipsychotic agent may be in the same pharmaceutically acceptable carrier and therefore administered simultaneously. They may be in separate pharmaceutical carriers such as conventional oral dosage forms which are taken simultaneously. The term “combination” is also to be understood to refer to the case where the compounds are provided in separate dosage forms and are administered sequentially.

It is further appreciated that when using a combination of the present invention, the CB2 receptor modulator and the other agent may be in the same pharmaceutically acceptable carrier and therefore administered simultaneously. They may be in separate pharmaceutical carriers such as conventional oral dosage forms which are taken simultaneously. The term “combination” is also to be understood to refer to the case where the compounds are provided in separate dosage forms and are administered sequentially.

As used herein, the term “treatment” generally refers to the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis, prevention) is also included.

In another embodiment, a pharmaceutical kit for use by a consumer to treat diseases, conditions or disorders by modulation or antagonism of the cannabinoid CB1 and/or CB2 receptor in an animal is described herein. The kit includes (a) a suitable dosage form of a compound of formula (I); and (b) instructions describing a method of using the dosage form to treat diseases, conditions or disorders by modulation or antagonism of the cannabinoid CB1 receptor. In another embodiment, a pharmaceutical kit is described herein that includes (a) a first dosage form comprising (i) a compound of formula (I) and (ii) a pharmaceutically acceptable carrier, excipient or diluent; and (b) a second dosage form comprising (i) an additional pharmaceutical agent described herein, and (ii) a pharmaceutically acceptable carrier, excipient or diluent; and (c) a container.

As used herein, the term “therapeutically-effective amount” generally refers to that amount of an active compound, or a material, composition or dosage from comprising an active compound, which is effective for producing some desired therapeutic effect, commensurate with a reasonable benefit/risk ratio, when administered in accordance with a desired treatment regimen.

As used herein, the term “agonist” generally refers to a compound (endogenous or exogenous, which may be a hormone, or synthetic compound) that binds to a receptor and mimics the effects of the endogenous regulatory compound, such as contraction, relaxation, secretion, change in enzyme activity, signal transduction, and the like. As used herein, the term “inverse agonist” generally refers to a compound that is functionally active at a receptor but produces the opposite effect produced by the agonist of the particular receptor. As used herein, the term “antagonist” generally refers to a compound, devoid of intrinsic regulatory activity, that produces effects by interfering with the binding of the endogenous agonist or inhibiting the action of an agonist. Further, the term “antagonist” refers to both full and/or partial antagonist. While a partial antagonist of any intrinsic activity may be useful, the partial antagonists illustratively show at least about 50% antagonist effect, or at least about 80% of the antagonist effect of a full antagonist. It is appreciated that illustrative methods described herein require therapeutically effective amounts of CB1 and/or CB2 receptor antagonists; therefore, compounds exhibiting partial antagonism at CB1 and/or CB2 receptors may be administered in higher doses to exhibit sufficient antagonist activity to achieve the desired therapeutic benefit.

Without being bound by theory, it is to be understood that the compounds described herein, including those of formulae (I)-(V), and the many variations, aspects, and alternative embodiments described herein, may modulate CB1 and/or CB2 receptor function by either reverse agonism or antagonism in displaying the therapeutic activity in the methods described herein.

The subject may be an animal, a chordate, a vertebrate, a mammal, a placenta mammal, a marsupial, such as kangaroo, wombat, a monotreme, such as duckbilled platypus, a rodent, such as a guinea pig, a hamster, a rat, a mouse, murine, such as a mouse, a lagomorph, such as a rabbit, avian, such as a bird, canine, such as a dog, feline, such as a cat, equine, such as a horse, porcine, such as a pig, ovine, such as a sheep, bovine, such as a cow, a primate, simian, such as a monkey or ape, a monkey, such as marmoset, baboon, an ape, such as gorilla, chimpanzee, orangutan, gibbon, or a human. Furthermore, the subject may be any of its forms of development, for example, an adult, an adolescent, a child, or a fetus. In one illustrative embodiment, the subject is a human.

The compounds and compositions may be administered by any route of administration, including but not limited to systemical, peripheral, or topical. Illustrative routes of administration include, but are not limited to, oral, such as by ingestion, buccal, sublingual, transdermal including, such as by a patch, plaster, and the like, transmucosal including, such as by a patch, plaster, and the like, intranasal, such as by nasal spray, ocular, such as by eyedrops, pulmonary, such as by inhalation or insufflation therapy using, such as via an aerosol through the mouth or nose, rectal, such as by suppository or enema, vaginal, such as by pessary, parenteral, such as by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and by implant of a depot or reservoir, such as intramuscularly.

Although the compounds described herein may be administered directly, the compounds described may also be formulated in any of a variety of ways that include at least one pharmaceutical acceptable carriers, diluents, excipients, adjuvants, fillers, buffers, preservatives, lubricants, solubilizers, surfactants wetting agents), masking agents, coloring agents, flavoring agents, and sweetening agents. As described herein, such formulation may also include other active agents, for example, other therapeutic or prophylactic agents.

Methods of making pharmaceutical composition include admixing at least one active compound, as defined above, together with one or more other pharmaceutically acceptable ingredients, such as carriers, diluents, excipients, and the like. If formulated as discrete units, such as tablets and the like, each unit contains a predetermined amount (dosage) of the active compound.

As used herein, the term “pharmaceutically acceptable” generally refers to compounds, ingredients, materials, compositions, dosage forms, etc. that are, within the scope of sound medical judgment, suitable for use in contact with the tissues of the subject in question, such as a human, without excessive toxicity, irritation, allergic response, or other problem or complication, commensurate with a reasonable ratio. Each carrier, diluent, excipient, etc. is also pharmaceutically acceptable in the sense of being compatible with the other ingredients of the formulation.

Suitable carriers, diluents, excipients, and other additives can be found in standard pharmaceutical texts, such as Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (liquid carriers, finely divided solid carrier, and the like), and then shaping the product, if necessary.

The formulation may be prepared to provide for rapid or slow release; immediate, delayed, timed, or sustained release; or a combination thereof. Formulations may suitably be in the form of liquids, solutions (aqueous, non-aqueous), suspensions (aqueous, non-aqueous), emulsions (oil-in-water, water-in-oil), elixirs, syrups, electuaries, mouthwashes, drops, tablets (including coated tablets), granules, powders, lozenges, pastilles, capsules (including hard and soft gelatin capsules), gels, pastes, ointments, creams, lotions, oils, foams, sprays, mists, or aerosols. Formulations may suitably be provided as a patch, adhesive plaster, bandage, dressing, or the like which is impregnated with one or more active compounds and optionally one or more other acceptable ingredients, including, for example, penetration, permeation, and absorption enhancers. Formulations may also suitably be provided in the form of depot or reservoir.

The active compound may be dissolved in, suspended in, or admixed with one or more other acceptable ingredients. The active compound may be presented in a liposome or other microparticulate which is designed to target the active compound, for example, to blood components or one or more organs. Formulations suitable for oral administration (e.g. by ingestion) include liquids, solutions (e.g. aqueous, non-aqueous), suspensions (e.g. aqueous, non-aqueous), emulsions (e.g. oil-in-water, water-in-oil), elixirs, syrups, electuaries, tablets, granules, powders, capsules, cachets, pills, ampoules, bouses. Formulations suitable for buccal administration include mouthwashes, lozenges, pastilles, as well as patches, adhesive plasters, depots, and reservoirs. Lozenges typically comprise the active compound in a flavored basis, usually sucrose and acacia or tragacanth. Pastilles typically comprise the active compound in an inert matrix, such as gelatin and glycerin, or sucrose and acacia. Mouthwashes typically comprise the active compound in a suitable liquid carrier. Formulations suitable for sublingual administration include tablets, lozenges, pastilles, capsules, and pills. Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, lozenges, pastilles, as well as patches, adhesive plasters, depots, and reservoirs, liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs. Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolat, cross-linked povidone, cross-linked sodium carboxymethyl cellulose); surface-active or dispersing or wetting agents (e.g. sodium lauryl sulfate); preservatives (e.g. methyl p-hydroxybenzoate, propyl p-hydroxybenzoate, sorbic acid); flavors, flavor enhancing agents, and sweeteners. Molded tablets may be made by molding in a suitable machine a mixture of the powdered compound moistened with an inert liquid diluent. The tablets may optionally be coated or scored and may be formulated so as to provide slow or controlled release of the active compound therein—using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base. Creams are typically prepared from the active compound and an oil-in-water cream base. If desired, the aqueous phase of the cream base may include, for example, at least about 30% of a polyhydric alcohol, such as propylene glycol, mannitol, sorbitol, glycerol and polyethylene glycol and mixtures thereof. The topical formulations may desirably include a compound which enhances absorption or penetration of the active compound through the skin or other affected areas. Examples of such penetration enhances include dimethylsulfoxide and related analogues.

Emulsions are typically prepared from the active compound and an oily phase, which may optionally comprise merely an emulsifier (otherwise known as an emulgent), or it may comprises a mixture of at least one emulsifier with a fat or an oil or with both a fat and an oil. Preferably, hydrophilic emulsifier is included together with lipophilic emulsifier which acts as stabilizer. It is also preferred to include both an oil and fat. Together, the emulsifier (s) with or without stabilizer(s) make up the so-called emulsifying wax, and the wax together with the oil and/or fat make up the so-called emulsifying ointment base which forms the oily dispersed phase of the cream formulations. Suitable emulgents and emulsion stabilizers include Tween 60, Span alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as paraffin-or-other mineral oils can be used.

Formulations suitable for intranasal administration, where the carrier is a liquid, include, for example, nasal spray, nasal drops, or by aerosol administration by nebuliser, include aqueous or oily solutions of the active compound. Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g. by inhalation or insufflation therapy) include those presented as an aerosol spray from a pressurized pack, with the use of a suitable propellant, such as dichoro-tetrafluoroethane, carbon dioxide, or other suitable gases. Formulations suitable for ocular administration include eye drops wherein the active compound is dissolved or suspended in a suitable carrier, especially an aqueous solvent for the active compound. Formulations suitable for rectal administration may be presented as suppository with a suitable base comprising, for example, natural or hardened oils, waxes, fats, semi-liquid or liquid polyols, for example, cocoa butter or a salicylate; or as a solution or suspension for treatment by enema. Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection), include aqueous or non-aqueous, isotonic, pyrogen-free, sterile liquids (e.g. solutions, suspensions), in which the active compound is dissolved, suspended, or otherwise provided (e.g. in a or other Such liquids may additional contain other pharmaceutical acceptable ingredients, such as anti-oxidants, buffers, preservatives, bacteriostats, suspending agents, thickening agents, and solutes which render the formulation isotonic with the blood (or other relevant bodily fluid) of the intended recipient.

Examples of excipients include, for example, water, alcohols, polyols, glycerol, vegetable oils, and the like. Examples of suitable isotonic carriers for use in such formulations include Sodium or Lactated Ringer's Injection. Typically, the concentration of the active compound in the liquid is from about 1 ng/mL to about 10 for example from about 10 ng/ml to about 1 The formulations may be presented in unit-dose or multi-dose sealed containers, for example, ampoules and vials, and may be stored in a freeze-dried condition requiring only the addition of the sterile liquid carrier, for example water for injections, immediately prior to use. Extemporaneous injection solutions and suspensions may be prepared from sterile powders, granules, and tablets.

It is appreciated that appropriate dosages of the cannabinoid receptor inverse agonists or cannabinoid receptor neutral antagonists, and compositions comprising them, can vary from patient to patient. Determining the optimal dosage will generally involve the balancing of the level of therapeutic benefit against any risk or deleterious side effects. The selected dosage level will depend on variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell (s) being treated, and the subject being treated. Single or multiple administrations can be carried out with the dose level and pattern being selected by the treating physician, veterinarian, or clinician.

In general, a suitable dose of the active compound is in the range of about 100 μg to about 250 mg (more typically about 100 μg to about 25 mg) per kilogram body weight of the subject per day. Where the active compound is a salt, an ester, an amide, a prodrug, or the like, the amount administered is calculated on the basis of the parent compound and so the-actual-weight-to-be-used is-increased-proportionately.

It has been reported by Rogers et al. “Bisphosphonates induce apoptosis in mouse macrophage-like cells in vitro by a nitric oxide-independent mechanism,” J. Bone Miner. Res. 11: 1482-1491 (1996), the disclosure of which is incorporated herein by reference, that inverse agonists can both block the action of the agonist and attenuate receptor constitutive activity of the cannabinoid CB1 receptors and have potent inhibitory effects on survival of macrophages, an established model system to test for compounds which inhibit osteoclastic activity. Such attenuated receptor activity affects the survival and resorptive activity of isolated rabbit osteoclasts, suggesting that inverse agonists of cannabinoid receptors have inhibitory effects on bone resorption, and identifies the endocannabinoid system as a therapeutic target for the treatment of bone diseases. In one aspect, the assay includes J774 murine macrophage viability, isolated rabbit osteoclast culture survival, or an osteoblast bone marrow assay. In another aspect, the assay includes ovariectomy-induced bone loss in a mouse model.

It has also been reported by Rogers et al. that inverse agonists can both block the action of the agonist and attenuate receptor constitutive activity of the cannabinoid CB2 receptors and have potent inhibitory effects on survival of macrophages, an established model system to test for compounds which inhibit osteoclastic activity. Such attenuated receptor activity affects the survival and resorptive activity of isolated rabbit osteoclasts, suggesting that inverse agonists of cannabinoid receptors have inhibitory effects on bone resorption, and identifies the endocannabinoid system as a therapeutic target for the treatment of bone diseases. In one aspect, the assay includes J774 murine macrophage viability, isolated rabbit osteoclast culture survival, or an osteoblast bone marrow assay. In another aspect, the assay includes ovariectomy-induced bone loss in a mouse model.

In one embodiment, the cannabinoid receptor inverse agonist or a cannabinoid receptor neutral antagonist is selective for CB1. In another embodiment, the cannabinoid receptor inverse agonist or a cannabinoid receptor neutral antagonist is selective for CB2. In another embodiment, the cannabinoid receptor inverse agonist or a cannabinoid receptor neutral antagonist exhibits activity at both CB1 and CB2.

In one embodiment of the invention, modulators of CB1 and/or CB2 receptor activation are described herein. Those modulators are of the general formula:

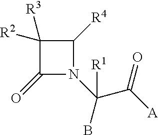

including selected enantiomers, diastereomers, and stereoisomeric mixtures thereof, and pharmaceutically acceptable salts, hydrates, solvates, and polymorphs thereof, wherein:

A is —OH or —NH₂; or A is taken together with the attached carbonyl group to form an ester or an amide;

B is a carboxylic acid, or an ester or amide derivative thereof; or B is alkyl, arylalkyl, hydroxyalkyl, alkylthiol, arylhydroxyalkyl, arylalkylthiol, aminoalkyl, or acyl, each of which is optionally substituted, or a derivative thereof, including ethers, esters, amides, carbonates, carbamates, ureas, ketals, and the like;

R¹ is hydrogen or C₁-C₆ alkyl;

R² is hydrogen, alkyl, alkenyl, alkynyl, alkoxy, alkylthio, halo, haloalkyl, cyano, formyl, alkylcarbonyl, or a substituent selected from the group consisting of —CO₂R⁸, —CONR⁸R^{8′}, and —NR⁸(COR⁹); where R⁸ and R^{8′} are each independently selected from hydrogen, alkyl, cycloalkyl, optionally substituted aryl, or optionally substituted arylalkyl; or R⁸ and R^{8′} are taken together with the attached nitrogen atom to form a heterocyclyl group; and where R⁹ is selected from hydrogen, alkyl, cycloalkyl, alkoxyalkyl, optionally substituted aryl, optionally substituted arylalkyl, optionally substituted heteroaryl, optionally substituted heteroarylalkyl, and R⁸R^{8′}N—(C₁-C₄ alkyl);

R³ is an amino, amido, acylamido, or ureido group, which is optionally substituted; or R³ is a nitrogen-containing heterocyclyl group attached at a nitrogen atom; or R³ is an optionally substituted aryl group; and

R⁴ is alkyl, alkenyl, alkynyl, cycloalkyl, cycloalkenyl, alkylcarbonyl, optionally substituted aryl, optionally substituted arylalkyl, optionally substituted arylhaloalkyl, optionally substituted arylalkoxyalkyl, optionally substituted arylalkenyl, optionally substituted arylhaloalkenyl, or optionally substituted arylalkynyl.

R⁸ and R^{8′} are each independently selected from hydrogen, alkyl, including C₁-C₆ alkyl, cycloalkyl, including C₃-C₈ cycloalkyl, optionally substituted aryl, or optionally substituted arylalkyl, including aryl(C₁-C₄ alkyl); or R⁸ and R^{8′} are taken together with the attached nitrogen atom to form an heterocycle, such as optionally substituted pyrrolidinyl, piperidinyl, morpholinyl, piperazinyl, and homopiperazinyl; and

R⁹ is selected from hydrogen, alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including aryl(C₁-C₄ alkyl), optionally substituted heteroaryl, optionally substituted heteroarylalkyl, including heteroaryl(C₁-C₄ alkyl), and R⁸R^{8′}N—(C₁-C₄ alkyl).

In another illustrative embodiment of the methods described herein, one or more compounds of formula (I):

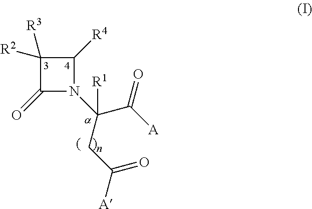

and pharmaceutically acceptable salts thereof, are administered to the patient; wherein

A and A′ are each independently selected from —OH and —NH₂; or A and/or A′ are taken together with the attached carbonyl group to form an ester or an amide;

n is an integer selected from 0 to about 3;

R¹ is hydrogen or C₁-C₆ alkyl;

R² is hydrogen, alkyl, alkenyl, alkynyl, alkoxy, alkylthio, halo, haloalkyl, cyano, formyl, alkylcarbonyl, or a substituent selected from the group consisting of —CO₂R⁸, —CONR⁸R^{8′}, and —NR⁸(COR⁹); where R⁸ and R^{8′} are each independently selected from hydrogen, alkyl, cycloalkyl, optionally substituted aryl, or optionally substituted arylalkyl; or R⁸ and R^{8′} are taken together with the attached nitrogen atom to form an heterocycle; and where R⁹ is selected from hydrogen, alkyl, cycloalkyl, alkoxyalkyl, optionally substituted aryl, optionally substituted arylalkyl, optionally substituted heteroaryl, optionally substituted heteroarylalkyl, and R⁸R^{8′}N—(C₁-C₄ alkyl);

R³ is an amino, amido, acylamido, or ureido group, which is optionally substituted; or R³ is a nitrogen-containing heterocyclyl group attached at a nitrogen atom; or R³ is an optionally substituted aryl group; and

R⁴ is alkyl, alkenyl, alkynyl, cycloalkyl, cycloalkenyl, alkylcarbonyl, optionally substituted aryl, optionally substituted arylalkyl, optionally substituted arylhaloalkyl, optionally substituted arylalkoxyalkyl, optionally substituted arylalkenyl, optionally substituted arylhaloalkenyl, or optionally substituted arylalkynyl.

In one illustrative aspect of the compounds of formula (I), A′ is selected from alkylamino, cycloalkylamino, cycloalkyl-alkylamino, arylamino, arylalkylamino, including aryl(C₁-C₂ alkyl)amino, indanylamino, and the like, each of which is optionally substituted, such as with halo, alkyl, alkoxy, alkylenedioxy, phenoxy, haloalkyl, haloalkoxy, and the like. In another aspect of the compounds of formula (I), A′ is a heterocycle or heterocyclylamino, such as pyrollidinyl, piperidinyl, and piperazinyl, or pyrollidinylamino, piperidinylamino, and piperazinylamino, that is substituted a group selected from alkyl, cycloalkyl, benzyl, benzimidazolyl, benzimidazolinonyl, 1-acetyl, and derivatives thereof, and the like. In one variation, A′ is piperidinyl or piperazinyl, or piperidinylamino or piperazinylamino, and the substituent is attached at the 4-position.

In another illustrative aspect, n is 1 or 2. In another illustrative aspect, n is 2. In another illustrative aspect, the stereochemistry at the α-carbon is (S).

In another illustrative embodiment of the methods described herein, one or more compounds of formula (II):

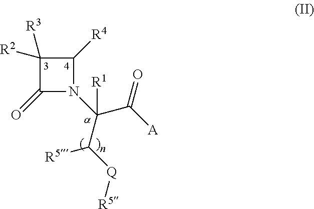

and pharmaceutically acceptable salts thereof, are administered to the patient; wherein

A is —OH or —NH₂; or A is taken together with the attached carbonyl group to form an ester or an amide;

Q is oxygen; or Q is sulfur or disulfide, or an oxidized derivative thereof;

n is an integer from 1 to 3;

R¹, R², R³, and R⁴ are as defined in formula I;

R^{5″} is selected from hydrogen, alkyl, cycloalkyl, alkoxyalkyl, optionally substituted arylalkyl, optionally substituted heterocyclyl or optionally substituted heterocyclylalkyl, and optionally substituted aminoalkyl; and

R^{5′″} is selected from hydrogen, alkyl, and optionally substituted arylalkyl.

In one aspect of the compounds of formula (II), R^{5″} is selected from alkyl, cycloalkyl, alkoxyalkyl, and optionally substituted arylalkyl; and R^{5′″} is selected from hydrogen and lower alkyl, such as methyl. In another aspect of the compounds of formula (II), R^{5″} is hydrogen or lower alkyl, such as methyl; and R^{5′″} is alkyl or optionally substituted arylalkyl.

In another illustrative embodiment of the methods described herein, one or more compounds of formula (III):

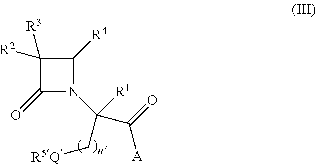

and pharmaceutically acceptable salts thereof, are administered to the patient; wherein

n is an integer in the range from about 1 to about 5, and is illustratively 1, 2, or 3;

A is —OH or —NH₂; or A is taken together with the attached carbonyl group to form an ester or an amide;

Q′ is oxygen or sulfur;

R¹, R², R¹, and R⁴ are as defined in formula I;

R^{5′} is selected from the group consisting of C₁-C₆ alkyl, C₃-C₈ cycloalkyl, (C₁-C₄ alkoxy)-(C₁-C₄ alkyl), optionally-substituted aryl(C₁-C₄ alkyl), Y′-(C₁-C₄ alkyl), where Y′- is a heterocycle, and R^{6′}R⁷N—(C₂-C₄ alkyl); where Y′ is selected from the group consisting of tetrahydrofuryl, morpholinyl, pyrrolidinyl, piperidinyl, piperazinyl, homopiperazinyl, or quinuclidinyl; where said morpholinyl, pyrrolidinyl, piperidinyl, piperazinyl, homopiperazinyl, or quinuclidinyl is optionally N-substituted with C₁-C₄ alkyl or optionally-substituted aryl(C₁-C₄ alkyl);

R^{6′} is hydrogen or alkyl, including C₁-C₆ alkyl, and R^{7′} is alkyl, including C₁-C₆ alkyl, cycloalkyl, including C₃-C₈ cycloalkyl, optionally substituted aryl, or optionally substituted arylalkyl, including aryl(C₁-C₄ alkyl); or R^{6′} and R^{7′} are taken together with the attached nitrogen atom to form an heterocycle, such as pyrrolidinyl, piperidinyl, morpholinyl, piperazinyl, and homopiperazinyl; where said piperazinyl or homopiperazinyl is optionally N-substituted with R^{13′}; and

R^{13′} is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

In another illustrative embodiment of the methods described herein, one or more compounds of formula (IV):

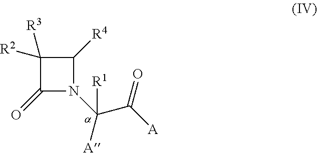

and pharmaceutically acceptable salts thereof, are administered to the patient; wherein

A is —OH or —NH₂; or A is taken together with the attached carbonyl group to form an ester or an amide;

A″ is hydrogen, halo, alkyl, cycloalkyl, aryl, heteroaryl, arylalkyl, heteroarylalkyl, aminoalkyl or a derivative thereof, alkylcarbonyl, cycloalkylcarbonyl, arylcarbonyl, heteroarylcarbonyl, arylalkylcarbonyl, or heteroarylalkylcarbonyl, each of which may be optionally substituted; and where the carbonyl of each is optionally an alkylene, arylalkylene, or heteroarylalkylene ketal; and

R¹, R², R³, and R⁴ are as defined in formula (I).

In one illustrative aspect of the compounds of formula (IV), A″ is hydrogen. In another aspect, A″ is hydrogen and A is alkoxy or arylalkoxy, each of which is optionally substituted. In another aspect, A″ is hydrogen, A is alkoxy or arylalkoxy, each of which is optionally substituted, and R³ is optionally substituted aryl, such as phenyl or thienyl. In another aspect, A″ is an alkyl, arylalkyl, or heteroarylalkyl corresponding to a naturally occurring aminoacid side chain, including but not limited to methyl, isopropyl, isobutyl, benzyl, 4-hydroxybenzyl, indolylmethyl, and the like.

In another aspect of the compounds of formula (IV), A″ is an alkylcarbonyl, arylalkylcarbonyl, or heteroarylalkylcarbonyl. In one variation, the alkyl, arylalkyl, or heteroarylalkyl corresponds to a naturally occurring aminoacid side chain, including but not limited to methyl, isopropyl, isobutyl, benzyl, 4-hydroxybenzyl, indolylmethyl, and the like. In another variation, the carbonyl group is converted to the corresponding alkylene ketal, such as an ethylene or propylene ketal, or arylalkylene ketal, such as a phenylmethylene, tolylmethylene, anisylmethylene, or hydroxyphenylmethylene ketal. In another variation, A is alkoxy or arylalkoxy, each of which is optionally substituted.

In another illustrative aspect of the compounds of formula (IV), A″ is aminoalkyl corresponding to a naturally occurring aminoacid side chain, such as aminopropyl, or a derivative thereof, such as an amide, carbamate, or urea derivative. In one variation, the aminoalkyl derivative is an amide, carbamate, or urea derivative of aminopropyl.

In another illustrative embodiment of the methods described herein, one or more compounds of formula (V):

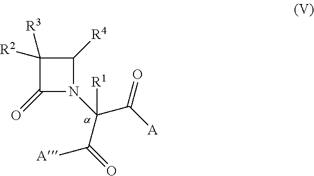

and pharmaceutically acceptable salts thereof, are administered to the patient; wherein A is a —CO₂H, or an ester or an amide derivative thereof; A′″ is alkyl, cycloalkyl, aryl, heteroaryl, arylalkyl, or heteroarylalkyl, each of which may be optionally substituted; and R¹, R², R³, and R⁴ are as defined in formula (I).

In one aspect of the compounds of formula (V), A′″ is branched alkyl, including isopropyl, sec-butyl, tert-butyl, neo-pentyl, and the like. In another variation, A is alkoxy or arylalkoxy, each of which is optionally substituted. In another variation, both R¹ and R² are hydrogen. In another aspect, R³ is optionally substituted aryl, including phenyl and thienyl, and the like. In one variation of the compounds of formula (V), the α-carbonyl is a ketal derivative, including an alkylene, arylalkylene, or heteroarylalkylene ketal, each of which may be optionally substituted.

In one aspect of the compounds of formulae (I)-(V), R¹ is hydrogen. In another aspect of the compounds of formulae (I)-(V), R¹ is methyl. In another aspect of the compounds of formulae (I)-(V), R² is hydrogen. In another aspect of the compounds of formulae (I)-(V), R² is methyl, ethyl, methoxy, methylthio, trifluoromethyl, trifluoromethoxy, cyano, or formyl.

In another aspect of the compounds of formulae (I)-(V), R³ is a structure selected from the group consisting of

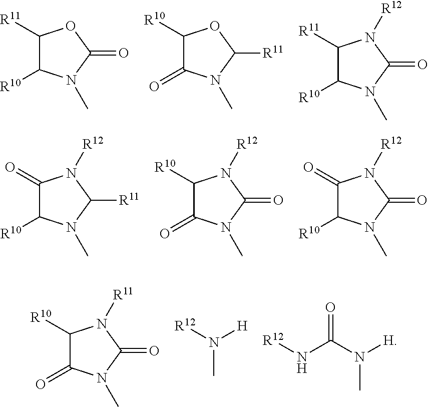

wherein R10 and R11 are each independently selected from hydrogen, optionally substituted alkyl, including C1-C6 alkyl, optionally substituted cycloalkyl, including C3-C8 cycloalkyl, alkoxyalkyl, including C1-C4 alkoxycarbonyl, alkylcarbonyloxy, including C1-C5 alkylcarbonyloxy, optionally substituted aryl, optionally substituted arylalkyl, including aryl(C1-C4 alkyl), optionally substituted arylalkyloxy, including aryl(C1-C4 alkyloxy), optionally substituted arylalkylcarbonyloxy, including aryl(C1-C4 alkylcarbonyloxy), diphenylmethoxy, and triphenylmethoxy; and

R¹² is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

In another embodiment of the compounds of formulae formulae (I)-(V), R³ is of the formulae:

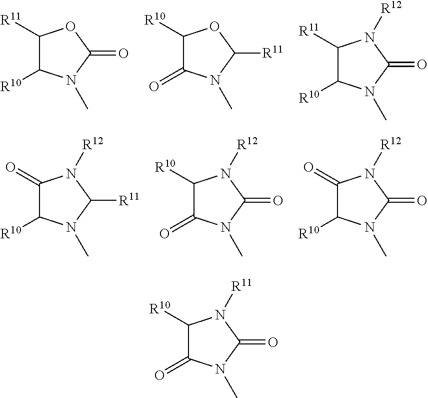

wherein R¹⁰, R¹¹, and R¹² are as defined herein.

In another embodiment of the compounds of formulae (I)-(V), R³ is of the formulae:

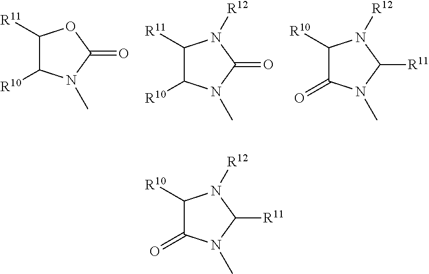

wherein R¹⁰, R¹¹, and R¹² are as defined herein.

In another embodiment of the compounds of formulae (I)-(V), R³ is of the formula:

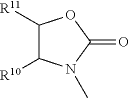

wherein R¹⁰ and R¹¹ are as defined herein.

In another embodiment of the compounds of formulae formulae (I)-(V), R³ is an optionally substituted aryl group, such as a phenyl, furanyl, thienyl, oxazolyl, isoxazolyl, thiazolyl, isothiazolyl, 1,2,3-triazolyl, 1,2,4-triazolyl, pyridinyl, pyridazinyl, pyrimidinyl, pyrazinyl, naphthyl, quinolinyl, quinazolinyl, quinoxalinyl, and the like, each of which is optionally substituted as described herein. In one variation, R³ is optionally substituted phenyl or optionally substituted thienyl.

In another embodiment of the compounds of formulae (I)-(V), R⁴ is aryl, arylalkyl, including aryl(C₁-C₄ alkyl), arylhaloalkyl, arylalkoxyalkyl, arylalkenyl, including aryl(C₂-C₄ alkenyl), arylhaloalkenyl, or arylalkynyl, including aryl(C₂-C₄ alkynyl), each of which may be optionally substituted. In one variation, R⁴ is of the formulae:

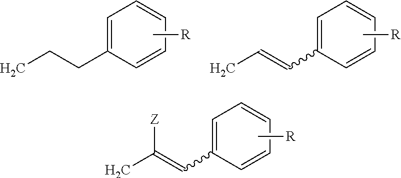

wherein Z an electron withdrawing group, such as halo, and R is hydrogen or from 1 to 3 optional substituents, including but not limited to halo, alkyl, and alkoxy, including 2-methoxy, and the like. In one aspect, Z is chloro. In another aspect, the double bond is (E).

In another embodiment of the compounds of formulae formulae (I)-(V), R⁴ is of the formulae:

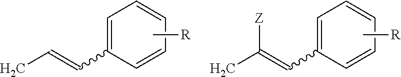

wherein Z is halo, and R is hydrogen. In one aspect, Z is bromo. In another aspect, the double bond is (E).

In another embodiment, compounds of formula

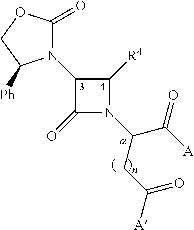

and pharmaceutically acceptable salts, hydrates, and solvates thereof are described, wherein R⁴ is substituted phenylethenyl, n is 1 or 2, and A and A′ are as defined herein.

In one aspect, the stereochemistry at C(3) is S. In another aspect, the stereochemistry at C(4) is R. In another aspect, the stereochemistry at C(α) is D when n is 1 and L when n is 2. In another aspect, A is not 3-trifluoromethylbenzyl. In another aspect, A is (R)-1,2,3,4-tetrahydronaphth-1-ylamino. In another aspect, A′ is a 4-substituted heterocycle, such as piperidinyl or piperazinyl. In another aspect, A′ is 4-cyclohexylpiperazinyl. In another aspect, A′ is 4-(piperidinyl)piperidinyl. In another aspect, R⁴ is phenylethenyl substituted with one or more substitutents selected from F, Cl, OMe, SMe, NO₂, CN, CF₃, and OCF₃.

In another embodiment, compounds of formula

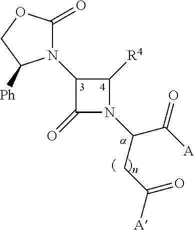

and pharmaceutically acceptable salts, hydrates, and solvates thereof are described, wherein R⁴ is optionally substituted heteroarylethenyl, n is 1 or 2, and A and A′ are as defined herein.

In one aspect, the stereochemistry at C(3) is S. In another aspect, the stereochemistry at C(4) is R. In another aspect, the stereochemistry at C(α) is L and n is 2. In another aspect, A is (R)-1,2,3,4-tetrahydro-1-naphthylamino. In another aspect, A′ is a 4-substituted heterocycle, such as piperidinyl or piperazinyl. In another aspect, A′ is 4-cyclohexylpiperazinyl. In another aspect, R⁴ is fur-2-yl. In another aspect, R⁴ is thien-2-yl. In another aspect, R⁴ is benzothiophen-7-yl.

In another embodiment of the compounds of formulae (I)-(V), A and/or A′ is —OR⁵, and forms an ester, where R⁵ is independently selected in each instance from alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted arylalkyl, including aryl(C₁-C₄ alkyl), Y—, Y—(C₁-C₄ alkyl), and R⁶R⁷N—(C₂-C₄ alkyl); where Y is a heterocycle, including tetrahydrofuryl, morpholinyl, pyrrolidinyl, piperidinyl, piperazinyl, homopiperazinyl, and quinuclidinyl; where said morpholinyl, pyrrolidinyl, piperidinyl, piperazinyl, homopiperazinyl, or quinuclidinyl is optionally N-substituted with alkyl, including C₁-C₄ alkyl or optionally substituted arylalkyl, including aryl(C₁-C₄ alkyl);

R⁶ is hydrogen or alkyl, including C₁-C₆ alkyl; and R⁷ is alkyl, including C₁-C₆ alkyl, cycloalkyl, including C₃-C₈ cycloalkyl, optionally substituted aryl, or optionally substituted arylalkyl, including aryl(C₁-C₄ alkyl); or R⁶ and R⁷ are taken together with the attached nitrogen atom to form an heterocycle, such as pyrrolidinyl, piperidinyl, morpholinyl, piperazinyl, and homopiperazinyl; where said piperazinyl or homopiperazinyl is optionally N-substituted with R¹³; and

R¹³ is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

In another aspect of the compounds of formulae (I)-(V), A and/or A′ are each an independently selected monosubstituted amino. In another aspect of the compounds of formulae (I)-(V), A and/or A′ are each an independently selected acyclic disubstituted amino. In another aspect of the compounds of formulae (I)-(V), A and/or A′ are each an independently selected cyclic disubstituted amino.

In another aspect of the compounds of formulae (I)-(V), A and/or A′ are each an independently selected monosubstituted amino having the formula XNH— where X is independently selected in each instance from the group consisting of alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including optionally substituted aryl(C₁-C₄ alkyl), and a group Y, Y—(C₁-C₄ alkyl), R⁶R⁷N—, and R⁶R⁷N—(C₂-C₄ alkyl), where Y is an heterocycle.

In another aspect of the compounds of formulae (I)-(V), A and/or A′ are each an independently selected acyclic disubstituted amino having the formula R¹⁴XN—; where R¹⁴ is independently selected in each instance from the group consisting of hydroxy, alkyl, including C₁-C₆ alkyl, alkoxycarbonyl, including C₁-C₄ alkoxycarbonyl, and benzyl; and where X is independently selected in each instance from the group consisting of alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including optionally substituted aryl(C₁-C₄ alkyl), and a group Y, Y—(C₁-C₄ alkyl), R⁶R⁷N—, and R⁶R⁷N—(C₂-C₄ alkyl), where Y is an heterocycle.

In another aspect of the compounds of formulae (I)-(V), A and/or A′ are each an independently selected cyclic disubstituted amino having the formula R¹⁴XN—; where R¹⁴ and X are taken together with the attached nitrogen atom to form an independently selected heterocycle, such as an heterocycle selected from the group consisting of pyrrolidinyl, piperidinyl, piperazinyl, and homopiperazinyl; where the heterocycle is optionally substituted with R¹⁰, R¹², R⁶R⁷N—, or R⁶R⁷N—(C₁-C₄ alkyl), where R⁶, R⁷, R¹⁰, and R¹² are as defined herein.

In another aspect of the compounds of formulae (I)-(V), A and/or A′ are each an independently selected from piperidinyl optionally substituted at the 4-position with hydroxy, alkyl, including C₁-C₆ alkyl, cycloalkyl, including C₃-C₈ cycloalkyl, alkoxy, including C₁-C₄ alkoxy, alkoxycarbonyl, including (C₁-C₄ alkoxy)carbonyl, hydroxyalkyloxyalkyl, including (hydroxy(C₂-C₄ alkyloxy))—(C₂-C₄ alkyl), R⁶R⁷N—, R⁶R⁷N-alkyl, including R⁶R⁷N—(C₁-C₄ alkyl), diphenylmethyl, optionally substituted aryl, optionally substituted aryl(C₁-C₄ alkyl), or piperidin-1-yl(C₁-C₄ alkyl).

In another aspect of the compounds of formulae (I)-(V), A and/or A′ are each an independently selected from piperazinyl optionally substituted at the 4-position with alkyl, including C₁-C₆ alkyl, cycloalkyl, including C₃-C₈ cycloalkyl, optionally substituted aryl, optionally substituted arylalkyl, including optionally substituted aryl(C₁-C₄ alkyl), α-methylbenzyl, and the like, N-alkyl acetamid-2-yl, including N—(C₁-C₅ alkyl)acetamid-2-yl, N-(cycloalkyl)acetamid-2-yl, including N—(C₃-C₈ cycloalkyl)acetamid-2-yl, R⁶R⁷N—, or alkoxycarbonyl, including (C₁-C₄ alkoxy)carbonyl.

In another aspect of the compounds of formulae (I)-(V), A and/or A′ are each an independently selected from homopiperazinyl optionally substituted in the 4-position with alkyl, including C₁-C₄ alkyl, aryl, or aryl(C₁-C₄ alkyl). In another aspect of the compounds of formulae (I)-(V), A and/or A′ are each an independently selected from pyrrolidinonyl, piperidinonyl, 2-(pyrrolidin-1-ylmethyl)pyrrolidin-1-yl, 1,2,3,4-tetrahydroisoquinolin-2-yl.

In another embodiment, compounds of formula (I)-(V) are described where A is selected from arylalkylamino, including aryl(C₁-C₂ alkyl)amino, 3-cycloalkylpyrollidinyl, 4-cycloalkylpiperidinyl, 4-cycloalkylpiperazinyl, tetrahydroquinolinyl, and tetrahydroisoquinolinyl, each of which is optionally substituted. Optional substituents are illustratively selected from halo, alkyl, alkoxy, alkylenedioxy, haloalkyl, haloalkoxy, and the like.

It is also to be understood that those compounds of formula (I) that include a carbon-carbon double bond, the double bond geometry may have either the E or Z configuration, or the compound may be a mixture of E or Z geometric isomers.

It is also to be understood that those compounds that include functional groups that may exist in two or more tautomeric forms, such ketones and the corresponding enol forms, also known as keto-enol tautomers, the individual tautomers as well as the mixtures thereof are collectively encompassed by the chemical formulae used herein.

In embodiments described herein that include enantiomeric or diastereomeric mixtures, such mixtures may be separated into pure enantiomers or diastereomers using conventional techniques, including fractional crystallization from a suitable solvent, by using an optically active resolving agent, such as a optically active amine or carboxylic acid to form a chiral resolvable salt, by chiral HPLC on a suitable chiral column, and others. Alternatively, it is to be understood that the syntheses described herein may be modified to prepare optically active intermediates or final products in a stereoselective or stereospecific manner.

In another embodiment, the compounds described herein may exist in alternate crystalline forms, such as polymorphs, each of which is also contemplated herein. In another embodiment, the compounds described herein may exist in alternate hydrated or solvated forms, with water or common organic solvents, each of which is also contemplated herein.

It is also appreciated that while the racemic form may be used without limitation, it may be generally preferable to the administer compounds of formula (I) in formulations having high enantiomeric purity.

As used herein, the term “pharmaceutically acceptable salt” generally refers to a to salts prepared from pharmaceutically acceptable non-toxic bases or acids including inorganic or organic bases and inorganic or organic acids. Salts derived from inorganic bases can be chosen from aluminum, ammonium, calcium, copper, ferric, ferrous, lithium, magnesium, manganic salts, manganous, potassium, sodium, zinc, and the like, such as for example, ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N, diethylamine, 2-diethylaminoethanol, 2-dimethylaminoethanol, ethanolamine, ethylenediamine, N-ethyl-morpholine, N-ethylpiperidine, glucamin, glucosamine, histidine, hydrabamine, isopropylamine, lysine, methylglucamine, morpholine, piperazine, piperidine, polyamine resins, procaine, purines, triethylamine, tripropylamine, tromethamine, and the like. The term further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

In another embodiment, pharmaceutical compositions comprising one or more compounds of formulae (I)-(V), including the many variations, aspects, and alternative embodiments described herein, and one or more pharmaceutically acceptable excipients, diluents, and/or carriers are described herein. Illustratively, the pharmaceutical compositions comprise a therapeutically effective amount of the one or more compounds of formula (I)-(V).

In another embodiment, pharmaceutical compositions are described herein, where the pharmaceutical compositions include one or more of the compounds described herein, including but not limited to the compounds of formulae (I)-(V), substituted 2-(azetidin-2-on-1-yl)alkanedioic acids, substituted 2-(azetidin-2-on-1-yl)hydroxyalkylalkanoic acids, substituted 2-(azetidin-2-on-1-yl)hydroxyalkylalkanoic acids, and/or substituted 2-(azetidin-2-on-1-yl)alkylalkanoic acids, including analogs and derivatives thereof described herein, and combinations thereof. The substituted 2-(azetidin-2-on-1-yl)alkanedioic acids, substituted 2-(azetidin-2-on-1-yl)hydroxyalkylalkanoic acids, substituted 2-(azetidin-2-on-1-yl)alkylalkanoic acids, and derivatives thereof include compounds of formulae (I)-(V). The pharmaceutical compositions described herein also include one or more pharmaceutically acceptable carriers, diluents, and/or excipients. In one illustrative aspect, pharmaceutical compositions are described that exhibit oral activity and/or oral bioavailability. In another illustrative aspect, pharmaceutical compositions are described that allow the substituted 2-(azetidin-2-on-1-yl)alkanedioic acids, substituted 2-(azetidin-2-on-1-yl)hydroxyalkylalkanoic acids, substituted 2-(azetidin-2-on-1-yl)alkylalkanoic acids, and analogs and derivatives thereof to cross the blood brain barrier.

It is to be understood that when used in combination as described hereinabove, such combination therapy may be administered as (a) a single pharmaceutical composition that includes one or more compounds of formulae (I)-(V), an additional pharmaceutical agent described herein, and a pharmaceutically acceptable excipient, diluent, or carrier; or (b) two separate pharmaceutical compositions where the first is a one or more compounds of formulae (I)-(V), and a pharmaceutically acceptable excipient, diluent, or carrier; and the second is an additional pharmaceutical agent described herein, and a pharmaceutically acceptable excipient, diluent, or carrier. In this latter variation, the pharmaceutical compositions may be administered simultaneously, contemporaneously, or separated in time according to any number of a variety of predetermined dosing regimens or protocols. In addition, the two compositions may be administered in either order as determined by the predetermined dosing regimen or protocol.

The compounds described herein may be prepared by general synthetic organic reactions. In particular, the compounds described herein may be prepared by methods described in U.S. Pat. Nos. 6,610,680, 6,521,611, 6, 204,260, and in U.S patent application publication 2004/0266750, and additional bond forming reactions are described in Richard C. Larock, “Comprehensive Organic Transformations, a guide to functional group preparations,” VCH Publishers, Inc. New York (1989); the disclosures of each of which are incorporated herein by reference.

### EXAMPLES

The following reagent abbreviations are used herein: BSA—bovine serum albumin; DMSO—dimethylsulfoxide EDTA—ethylenediamine tetracetic acid; PBS—phosphate-buffered saline; EGTA—ethylene glycol-ib/s(β-aminoethyl ether); N,N,N′,N′-tetraacetic acid GDP—guanosine diphosphate; sc—subcutaneous; po—orally; ip—intraperitoneal; icv—intra cerebro ventricular; iv—intravenous; [³H]SRI 41716A-radiolabeled N-(piperidin-1-yl)-5-(4-chlorophenyl)-1-(2,4-dichlorophenyl)-4-methyl-1H-pyrazole-3-carboxamide hydrochloride available from Amersham Biosciences, Piscataway, N.J.; [³H]CP-55940-radiolabled 5-(1,1-dimethylheptyl)-2-[5-hydroxy-2-(3-hydroxypropyl)-cyclohexyl]-phenol available from NEN Life Science Products, Boston, Mass.; AM251-N-(piperidin-1-yl)-1-(2,4-dichlorophenyl)-5-(4-iodophenyl)-4-methyl-1H-pyrazole-3-carboxamide available from TOCRIS, Ellisville, Mo.

Cannabinoid receptor binding (and thus ligand affinity) can readily be determined by looking for displacement of a suitable known ligand by a test ligand from mouse brain and spleen membranes. Examples of suitable known ligands include tritium labeled SR141716A (a CB1-specific receptor inverse agonist) and tritium labeled CP55940 (a CB1/CB2 receptor agonist).

Cannabinoid Receptor-1 (CB1) Binding Assay on Cells.

Method A using cell membranes. A compound is dissolved in DMSO and diluted to 1 M as a stock solution. The cannabinoid receptor-1 agonist ³H—CP55940 (PerkinElmer Life Sciences, Inc, Boston, Mass.) is diluted with binding buffer (50 mM Tris-HCl at pH 7.5, 5 mM MgCl₂, 2.5 mM EDTA, 5 mg/ml BSA) to a working concentration of 2 nM. A CB1R-containing cell membrane (Applied Cell Sciences Inc, Rockville, Md.) is diluted with binding buffer to a protein concentration of 28 μg/ml. The following mixture is added to the CB1R-containing cell membrane: 200 μl containing stock solution of compound (2 μl), ³H-CP55940 (100 μl), and CB1R-containing cell membrane (98 μl). The final concentrations are compound (10 μM), and agonist CP55940 (1 nM), and amount of cell membrane protein of 2.74 μg per tube.

The mixture is incubated for 90 min at room temperature, then filtered through Whatman glass fiber C disc filter in a vacuum manifold. The filter is washed three times each with 2.5 ml cold wash buffer (binding buffer with only 2.5 mg/ml BSA). The vacuum-dried filter is transferred to a scintillation vial, treated with 3 ml Eco-Scint, and the radioactivity is read on a Beckman LS6500 scintillation counter from which an IC₅₀ value is calculated. The corresponding Kᵢ values are obtained from serial dilutions according to this method, and calculated through the use of a non-linear regression program (Graphpad Inc, San Diego, Calif.). The compounds described herein were tested according to this method, and selected data is presented in the following table:

| Example | IC₅₀ (μM) | Kᵢ (μM) |
|---|---|---|
| 20Q | <10 | — |
| 35AJ | <10 | — |
| 35AK | <10 | — |
| 37L | <10 | — |
| 37M | <10 | — |
| 37N | <10 | — |
| 37O | <10 | — |
| 37P | <10 | — |
| 37Q | <10 | — |
| 37R | <10 | — |
| 37S | 2.3 | 1.15 |
| 37T | <10 | — |
| 37U | 2.1 | 1.07 |
| 37V | <10 | — |
| 37W | <10 | — |
| 37AA | <10 | — |
| 37AB | <10 | — |
| 37AC | <10 | — |
| 37AD | <10 | — |
| 37AE | <10 | — |
| 38K | ~10 | — |
| 38L | ~10 | — |
| 38M | ~10 | — |
| 38N | ~10 | — |
| 38O | <10 | — |
| 38V | <10 | — |
| 38AC | <10 | — |
| 38AD | <10 | — |
| 38AE | <10 | — |
| 38AF | <10 | — |
| 38AG | <10 | — |
| 38BH | <10 | — |
| 38BL | <10 | — |
| 38BM | <10 | — |
| 44G | <10 | — |
| 53A | <10 | — |
| 70A | <10 | — |
| 70B | <10 | — |
| 70C | 1.4 | 0.67 |
| 71A | 1.19 | 0.595 |
| 71B | <10 | — |
| 71C | <10 | — |
| 71D | ~10 | — |
| 71E | ~10 | — |
| 71F | ~10 | — |
| 71H | <10 | — |
| 72A | ~10 | — |
| 72C | ~10 | — |
| 73A | <10 | — |
| 73B | ~10 | — |
| 73C | ~10 | — |
| 73D | ~10 | — |
| 73E | <10 | — |
| 73F | ~10 | — |
| 73G | ~10 | — |
| 73H | ~10 | — |

The K_{d} and the IC₅₀ values for ³H-CP55940 were about 1 nM, and about 2 nM, respectively. For about 2500 cpm of specific binding, a 5 to 1 ratio of signal to noise was observed.

Method B using CHO cells transfected with human CB1. Binding affinity determination is based on recombinant human receptor expressed in Chinese Hamster Ovary (CHO) cells (Felder et al, Mol. Pharmacol. 48:443-450, 1995). Total assay volume is 250 μL (240 μL CB1 receptor membrane solution plus 5 μL test compound solution plus 5 μL [3H] CP-55940 solution). Final concentration of [3H] CP-55940 is 0.6 nM. Binding buffer contains pH 7.4, 2.5 mM EDTA, 5 mM MgCl₂, 0.5 mg/μL fatty acid free bovine serum albumin and protease inhibitors (Cat#P8340, from Sigma). To initiate the binding reaction, 5 μL solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantified using a scintillation counter. Apparent binding affinities for various compounds are calculated from IC₅₀ values (DeBlasi et al., Trends Pharmacol Sci 10:227-229, 1989).

Method C using HEK cells transfected with human CB1. Human embryonic kidney 293 (HEK 293) cells transfected with the CB-1 receptor cDNA (obtained from Dr. Debra Kendall, University of Connecticut) are harvested in homogenization buffer (10 mM EDTA, 10 mM EGTA, 10 mM Na Bicarbonate, protease inhibitors; pH=7.4), and homogenized with a Dounce Homogenizer. The homogenate was then spun at 1,000×g for 5 minutes at 4° C. The supernatant was recovered and centrifuged at 25,000×G for 20 minutes at 4° C. The pellet was then re-suspended in 10 ml of homogenization buffer and re-spun at 25,000×G for 20 minutes at 4° C. The final pellet was re-suspended in 1 ml of TME (25 mM Tris buffer (pH=7.4) containing 5 mM MgCl₂ and 1 mM EDTA). A protein assay was performed and 200 g of tissue totaling 20 μg was added to the assay. The test compounds were diluted in drug buffer (0.5% BSA, 10% DMSO and TME) and then 25 μl were added to a deep well polypropylene plate. [3H] SR141716A was diluted in a ligand buffer (0.5% BSA plus TME) and 25 μl were added to the plate. The plates were covered and placed in an incubator at 30° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

Cannabinoid Receptor-2 (CB2) Binding Assay on Cells.

Method A using cell membranes. A compound is dissolved in DMSO and diluted to 1 M as a stock solution. The cannabinoid receptor-2 agonist ³H—CP55940 (PerkinElmer Life Sciences, Inc, Boston, Mass.) is diluted with binding buffer (50 mM Tris-HCl at pH 7.5, 5 mM MgCl₂, 2.5 mM EDTA, 5 mg/ml BSA) to a working concentration of 2 nM. A CB2R-containing cell membrane (Applied Cell Sciences Inc, Rockville, Md.) is diluted with binding buffer to a protein concentration of 28 μg/ml. The following mixture is added to the CB2R-containing cell membrane: 200 μl containing stock solution of compound (2 μl), ³H-CP55940 (100 μl), and CB2R-containing cell membrane (98 μl). The final concentrations are compound (10 μM), and agonist CP55940 (1 nM), and amount of cell membrane protein of 2.74 μg per tube.

The mixture is incubated for 90 min at room temperature, then filtered through Whatman glass fiber C disc filter in a vacuum manifold. The filter is washed three times each with 2.5 ml cold wash buffer (binding buffer with only 2.5 mg/ml BSA). The vacuum-dried filter is transferred to a scintillation vial, treated with 3 ml Eco-Scint, and the radioactivity is read on a Beckman LS6500 scintillation counter from which an IC₅₀ value is calculated. The corresponding Kᵢ values are obtained from serial dilutions according to this method, and calculated through the use of a non-linear regression program (Graphpad Inc, San Diego, Calif.). The compounds described herein were tested according to this method, and selected data is presented in the following table:

| Example | IC₅₀ (μM) | Kᵢ (μM) |
|---|---|---|
| 19E | <10 | — |
| 19F | <1 | — |
| 19H | <1 | — |
| 28A | <10 | — |
| 31A | 0.379 | 0.253 |
| 31I | ~1 | — |
| 33G | 0.273 | 0.182 |
| 42H | ~10 | — |
| 42I | ~10 | — |
| 42J | ~10 | — |
| 42K | ~10 | — |
| 42L | ~10 | — |
| 42M | ~10 | — |
| 44B | <1 | — |
| 44G | ~1 | — |
| 45U | <10 | — |
| 62B | 0.480 | 0.320 |
| 62E | 0.539 | 0.360 |
| 70B | ~1 | — |

The K_{d} and the IC₅₀ values for ³H-CP55940 were about 1 nM, and about 2 nM, respectively. For about 2500 cpm of specific binding, a 5 to 1 ratio of signal to noise was observed.

Method B using CHO cells transfected with human CB2. Binding affinity determination is based on recombinant human receptor expressed in Chinese Hamster Ovary (CHO) cells (Felder et al, Mol. Pharmacol. 48:443-450, 1995). Total assay volume is 250 μL (240 μL CB2 receptor membrane solution, 5 μL test compound solution, 5 μL [³H] CP-55940 solution). Final concentration of [³H] CP-55940 is 0.6 nM. Binding buffer contains pH 7.4, 2.5 mM EDTA, 5 mM MgCl₂, 0.5 mg/μL fatty acid free bovine serum albumin and protease inhibitors (Cat#P8340, from Sigma). To initiate the binding reaction, 5 μL solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantified using a scintillation counter. Apparent binding affinities for various compounds are calculated from IC₅₀ values (DeBlasi et al., Trends Pharmacol Sci 10:227-229, 1989).

Method C using HEK cells transfected with human CB2. Human embryonic kidney 293 (HEK 293) cells transfected with the CB-2 receptor cDNA (obtained from Dr. Debra Kendall, University of Connecticut) are harvested in homogenization buffer (10 mM EDTA, 10 mM EGTA, 10 mM Na Bicarbonate, protease inhibitors; pH=7.4), and homogenized with a Dounce Homogenizer. The homogenate is spun at 1,000×g for 5 minutes at 4° C. The supernatant was recovered and centrifuged at 25,000×G for 20 minutes at 4° C. The pellet was then re-suspended in 10 ml of homogenization buffer and re-spun at 25,000×G for 20 minutes at 4° C. The final pellet was re-suspended in 1 ml of TME (25 mM Tris buffer (pH=7.4) containing 5 mM MgCl₂ and 1 mM EDTA). A protein assay was performed and 200 μl of tissue totaling 20 μg was added to the assay. The test compounds were diluted in drug buffer (0.5% BSA, 10% DMSO and TME) and then 25 μl were added to a deep well polypropylene plate. The CB2 agonist ³H-CP55940 is diluted in a ligand buffer (0.5% BSA plus TME) and 25 μl were added to the plate. The plates were covered and placed in an incubator at 30° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

Cannabinoid Receptor Binding Assays on Tissue. MF1 mice are killed by cervical dislocation and the desired tissues (e.g. brain) dissected out and placed into cold centrifugation buffer (320 mM sucrose, 2 mM Tris EDTA, 5 mM MgCl2) on ice. Tissue is then homogenized with an ultra-turrax poltron homogeniser. The homogenate is centrifuged at 1600×g for 10 minutes, the supernatant saved on ice and the pellet re-suspended in cold centrifugation buffer and centrifuged at 1600×g for 10 minutes. The supernatants are combined and centrifuged at 32000×g for 20 minutes. This supernatant is discarded and 50 mL of Tris Buffer (50 mM Tris, 2 mM EDTA and 5 mM MgCl₂) incubated at 37° C. for 10 minutes and centrifuged at 23000×g for 20 minutes. The final pellet is resuspended in 40 mL Tris Buffer and left to stand at room temperature for 40 minutes. This solution is then centrifuged at 11000×g for 15 minutes and the pellet resuspended in assay buffer (1 mM MgCl2, 50 mM Tris, 1 mM EDTA) to a concentration of 1 mg/mL as determined by the Lowry method (Bio-Rad Dc kit).

Radioligand binding assays are performed, for example, with the CB1 receptor inverse agonist [³H] SR141716A (0.5 nM) or agonist [³H] CP55940 (0.5 nM) on brain membranes in assay buffer containing 1 mg/mL BSA, the total assay volume being 500 μL. Binding is initiated by the addition of membranes (100 □g). The vehicle concentration of 0.1% DMSO is kept constant throughout. Assays are carried out at 37° C. for 60 minutes before termination by addition of ice-cold wash buffer (50 mM Tris buffer, 1 mg/mL BSA) and vacuum filtration using a 12-well sampling manifold (Brandel Cell Harvester) and Whatman GF/B glass-fibre filters that had been soaked in wash buffer at 4° C. for 24 hours. Each reaction tube is washed five times with 4 mL aliquot of buffer. The filters are oven-dried for 60 minutes and then placed in 5 mL of scintillation fluid (Ultima Gold XR, and radioactivity by liquid scintillation spectrometry.

Further, radioligand binding assays are performed, for example, with the CB2 receptor agonist [³H] CP55940 (0.5 nM) (spleen membranes) in assay buffer containing 1 mg/mL BSA, the total assay volume being 500 μL. Binding is initiated by the addition of membranes (100 □g). The vehicle concentration of 0.1% DMSO is kept constant throughout. Assays are carried out at 37° C. for 60 minutes before termination by addition of ice-cold wash buffer (50 mM Tris buffer, 1 mg/mL BSA) and vacuum filtration using a 12-well sampling manifold (Brandel Cell Harvester) and Whatman GF/B glass-fibre filters that had been soaked in wash buffer at 4° C. for 24 hours. Each reaction tube is washed five times with 4 mL aliquot of buffer. The filters are oven-dried for 60 minutes and then placed in 5 mL of scintillation fluid (Ultima Gold XR, and radioactivity by liquid scintillation spectrometry.

Specific binding is defined as the difference between the binding that occurred in the presence and absence of 1 μM unlabelled ligand and reported as a percentage of the total radio-ligand bound in brain. The concentrations of competing ligands (test compounds) to produce 50% displacement of the radioligand (IC50) from specific binding sites is calculated, for example, using GraphPad Prism (GraphPad Software, San Diego). Inhibition constant (Ki) values are calculated using the equation of Cheng & (see, e.g., Cheng et 1973).

Additional details for performing assays for determining cannabinoid receptor affinity are described in Ross et 1999a; Ross et 1999b; Huffman et 2000; Huffman et 2001. For example, radio-ligand displacement assays using tissues that contain the CB1 receptor (brain, CB1 transfected cell lines) are common. Examples of suitable radio-labelled known (reference) ligands include receptor inverse agonist), tritium-labeled CP55940 (a receptor agonist), the disclosures of each of which are incorporated herein by reference.

Rat CB-1 Receptor Binding Protocol. PelFreeze brains (available from Pel Freeze Biologicals, Rogers, Ark.) were cut up and placed in tissue preparation buffer (5 mM Tris HCl, pH=7.4 and 2 mM EDTA), polytroned at high speed and kept on ice for 15 minutes. The homogenate was then spun at 1,000×G for 5 minutes at 4° C. The supernatant was recovered and centrifuged at 100,000×G for 1 hour at 4° C. The pellet was then re-suspended in 25 ml of TME (25 nM Tris, pH=7.4, 5 mM MgCl₂, and 1 mM EDTA) per brain used. A protein assay was performed and 200 μl of tissue totaling 20 μg was added to the assay. The test compounds were diluted in drug buffer (0.5% BSA, 10% DMSO and TME) and then 25 μl were added to a deep well polypropylene plate. [3H] SR141716A was diluted in a ligand buffer (0.5% BSA plus TME) and 25 μl were added to the plate. A BCA protein assay was used to determine the appropriate tissue concentration and then 200 μl of rat brain tissue at the appropriate concentration was added to the plate. The plates were covered and placed in an incubator at 20° C. for 60 minutes. At the end of the incubation period 250 μl of stop buffer (5% BSA plus TME) was added to the reaction plate. The plates were then harvested by Skatron onto GF/B filtermats presoaked in BSA (5 mg/ml) plus TME. Each filter was washed twice. The filters were dried overnight. In the morning the filters were counted on a Wallac Betaplate™ counter (available from PerkinElmer Life Sciences™, Boston, Mass.).

Cannabinoid Receptor-1 (CB1) Functional Activity Assay.

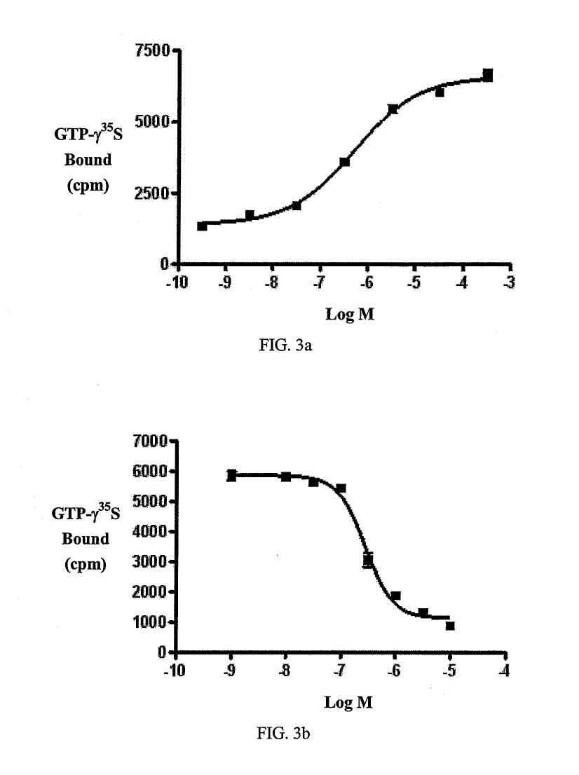

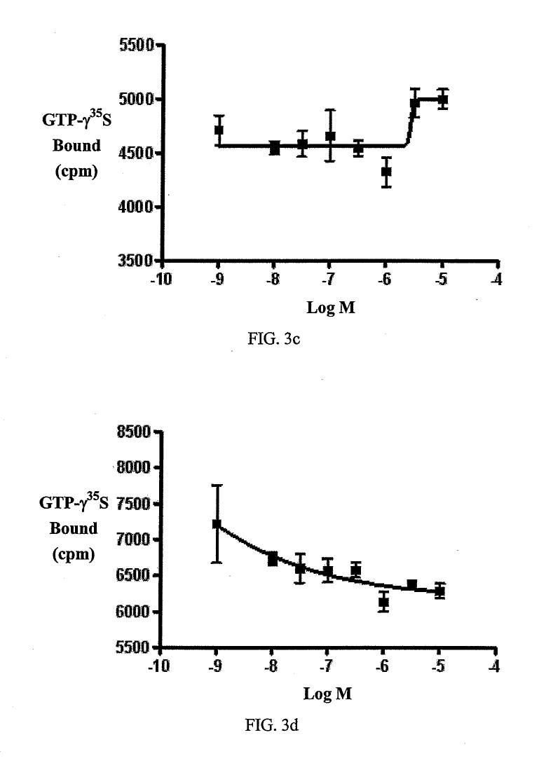

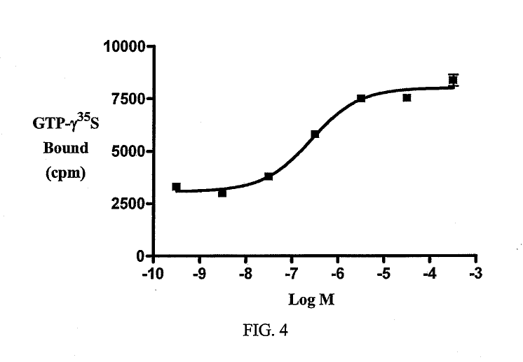

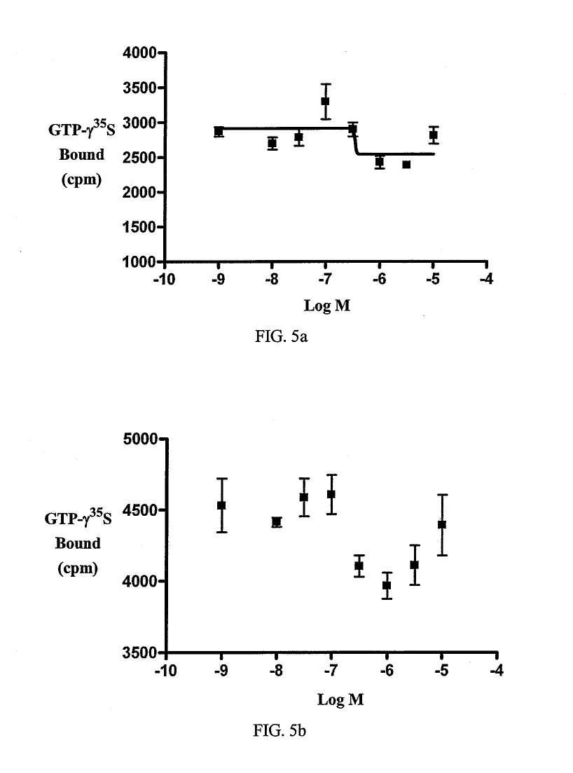

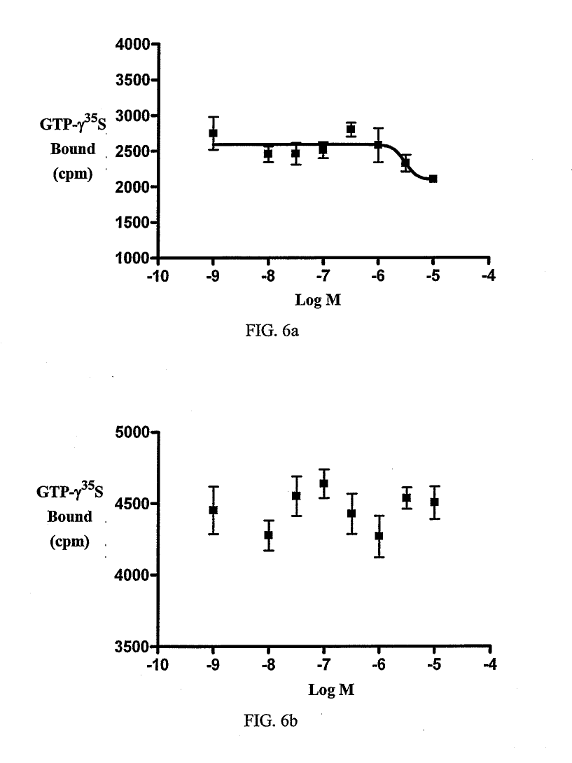

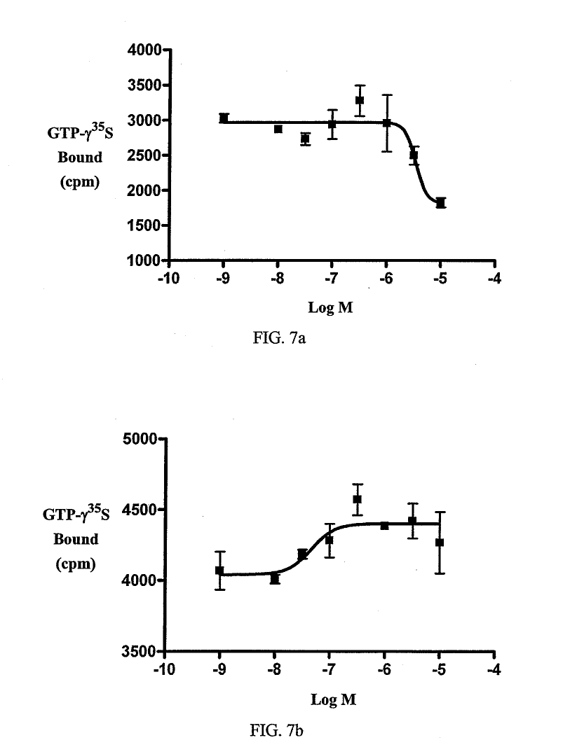

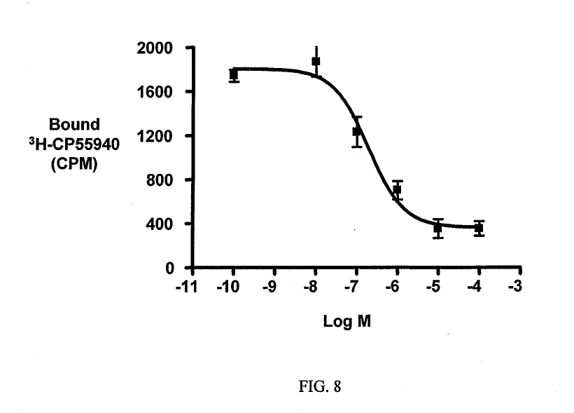

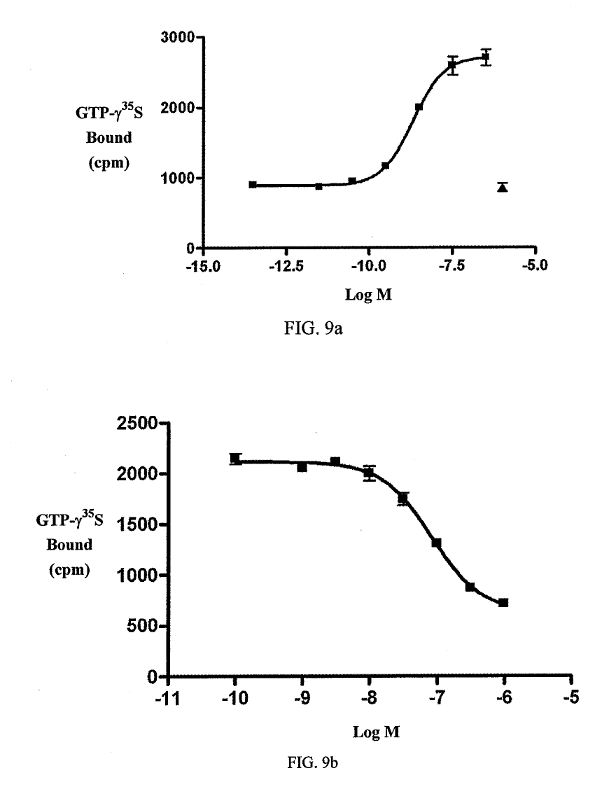

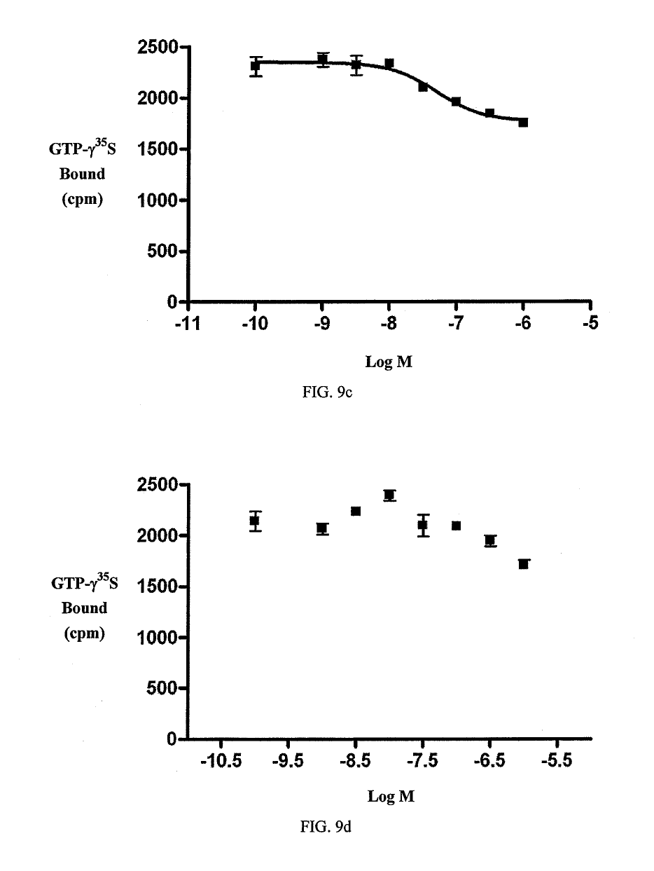

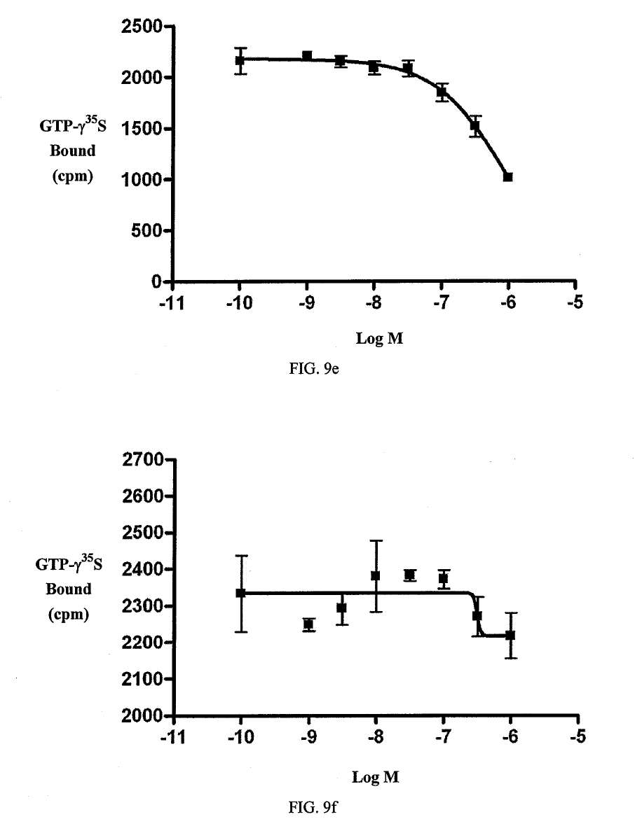

Method A. Competition with [³H]WIN 55212-2. The functional activity of the compounds described herein at the CB1 receptor is determined by the ability of test compounds to displace [³H]WIN 55212-2 from the receptor in membranes derived from hCB1 stably transfected CHO cells (Perkin Elmer/NEN). Membranes are diluted in ice-cold assay buffer (10 mM HEPES, 100 mM NaCl, 10 mM MgCl₂, 2 μM GDP, 0.2% FAF-BSA, 10 μg/mL saponin) to 38 μg membranes/mL. Dilute ³⁵S-GTPγ5 to 1 nM in assay buffer. A positive agonist control with 20 μL of [³H]WIN 55212-2 is performed from 3 nM to 300 μM, with 20 μL of ³⁵S-GTPγ5 and 50 μL of membranes. The compounds described herein (20 μL of 1 nM to 10 μM) are tested alone and then again against 20 μL of [³H]WIN 55212-2 at 30 μM, with 20 μL of ³⁵S-GTPγ5 and 50 μL of membranes. These solutions are incubated for 30 minutes at 30° C. The filters are washed 6 times with 10 mM sodium phosphate buffer (pH 7.4) at ambient temperature. The filters are counted using a liquid scintillation counter on the ³⁵S channel. Each compound/concentration is run in triplicate. A positive antagonism control may be run for AM251 at the same concentrations as test compounds under the same conditions. The results for several examples described herein are shown in FIGS. 3a-3d, and 4a-4-d.

Method B. Measurement of adenylyl cyclase production. Although binding studies measure the affinity of a ligand for the receptor, such studies do not indicate the functional characteristics of the ligand (that is, whether it acts as an agonist, neutral antagonist, inverse agonist, etc.). Thus, many cannabinoid receptor ligands may also be conveniently classified according to their functional characteristics, for example, their effect upon cannabinoid receptor activity, for example, as an agonist, neutral antagonist, inverse agonist, etc. The CB1 receptor (GPCR) super-family is coupled to inhibition of adenylyl cyclase and activation of signal-regulated cascade (ERK).

The traditional model of G protein-coupled receptor (GPCR) action is based on the premise that the binding of an agonist to the receptor is necessary for receptor activation. However, it is now clear that some receptor activation occurs spontaneously, without agonist binding, the receptors being constitutively active. Cannabinoid CB1 receptors appear to be constitutively active. A large body of evidence for this has been obtained from high expression recombinant cell lines where cannabinoid receptor inverse agonists stimulate adenylyl cyclase and inhibit ERK (see, e.g., Bouboula et; Bouboula et 1997; Bouboula et 1999).

By sequestration of Gi proteins, cannabinoid inverse agonists not only inhibit constitutively active CB1 receptors but also inhibit receptor activation by other unrelated Gi-dependent receptors. In general, ligands that do not bind directly to a receptor, but do affect the receptor's function, may be described are numerous examples of so-called allosteric modulators of G-protein coupled receptors that bind to a site closely related to the receptor and modulate the function of the receptor (see, e.g., Vaulquelin et 2002).

Such sites may exist for the cannabinoid receptors; however, none have yet been identified. Thus, many cannabinoid receptor ligands may be further classified as: (a) cannabinoid receptor agonists, which activate the receptor; partial agonists also activate the receptor, but with lower efficacy than full agonist; (b) cannabinoid receptor inverse agonists, which both block the action of the agonist and attenuate receptor-constitutive activity; (c) cannabinoid receptor neutral antagonists, which block the action of the agonist but are ineffective on the receptor-constitutive activity; they may also be low efficacy partial agonists that behave

The functional activation of receptor is based on recombinant human receptor expressed in CHO cells (Felder et al., Mol. Pharmacol. 48:443-450, 1995). To determine the agonist activity or inverse agonist activity of any test compound, 50 μL of CB1-CHO cell suspension are mixed with test compound and 70 μL assay buffer containing 0.34 mM 3-isobutyl-1-methylxanthine and 5.1 μM of forskolin in 96-well plates. The assay buffer is comprised of Earle's Balanced Salt Solution supplemented with 5 mM MgCl₂, 1 mM glutamin, 10 mM HEPES, and 1 mg/mL bovine serum albumin.

The mixture is incubated at room temperature for 30 minutes, and terminated by adding 30 μL/well of 0.5M HCl. The total intracellular cAMP level is quantified using the New England Nuclear Flashplate and cAMP radioimmunoassay kit. To determine the antagonist activity of test compound, the reaction mixture also contains 0.5 nM of the agonist CP55940, and the reversal of the CP55940 effect is quantified. Alternatively, a series of dose response curves for CP55940 is performed with increasing concentration of the test compound in each of the dose response curves.

Method C. Cyclic AMP Assay. Cannabinoid receptors CB1 and CB2 are coupled to inhibition of adenylyl cyclase (see, e.g., Bidault-Russell et 1990; et 1996). Adenylyl cyclase is an enzyme that catalyses the production of cyclic adenosine monophosphate (AMP). Thus, activation of the receptor leads to the inhibition of the production of cyclic AMP. Certain compounds, such as forskolin, stimulate adenylyl cyclase. Accumulation of cyclic AMP is then measured using a and is indicative of adenylyl cyclase activation. The amount of radioactivity can be measured and will be proportional to the level of cyclic AMP that is produced. The cyclic AMP assay is performed with a phosphodiesterase inhibitor present. This is necessary because phosphodiesterase is an enzyme that rapidly breaks down cyclic AMP. An example of a phosphodiesterase inhibitor is rolipram. The cyclic AMP assay is performed using cells that contain CB1 receptors only (Chinese Hamster Ovary Cells or Human Embryonic Kidney Cells).

The cells or tissues are incubated for 30 minutes at 37° C. with the cannabinoid receptor ligand and the phosphodiesterase inhibitor rolipram (Sigma) (50 μM) in phosphate buffered saline (PBS) containing 1 mg/ml bovine serum albumin (Sigma). The cells or tissues are then incubated for a further 30 minutes incubation with 1 μM forskolin (Sigma). The reaction is terminated by addition 0.1 M hydrochloric acid and the mixture is centrifuged in a to remove cell debris. The resulting pellet contains cell debris and the supernatant contains the [³H]cyclic AMP. A sample of a supernatant is removed and the pH is adjusted to pH using 1 M The cyclic AMP content is then measured using a radioimmunoassay kit ([³H] Biotrack assay TRK432, from Amersham Biosciences), following the manufacturers instructions. The amount of radioactivity in each sample is counted using a Beckman scintillation counter. The amount is cyclic AMP in each sample is calculated from the level of radioactivity.

Method D. [³⁵S]-γ-GTP Assay using cell homogenates. Activation of a G-protein coupled receptor by an agonist leads to the replacement of guanosine diphosphate (GDP) with guanosine triphosphate (GTP). The level of binding of GTP to the receptor is proportional to the level of receptor activation. The level of binding is measured by using a radiolabelled form of GTP called [³⁵S]-γ-GTP. Thus, the radioactivity can be measured and is proportional to the amount of GTP bound to the receptor. The [³⁵S]-γ-GTP binding assay is performed with cells that contain CB1 receptors only or cells that contain CB2 receptors only (Chinese Hamster Ovary cells or human embryonic kidney cells, respectively). The [³⁵S]-γ-GTP binding assay may also be performed with tissues that contain CB1 receptors (e.g., brain) or CB2 receptors (e.g., spleen).

Cells that contain CB1 receptors only are removed from flasks by scraping, and are re-suspended in homogenization buffer (0.32 M sucrose/50 mM Tris), and using an Ultra-Turrex. If tissues are used, the homogenate is prepared as for a radioligand binding assay, as described herein. The homogenate is diluted with Tris buffer (50 mM, pH 7.4) and centrifuged at 50,000 times g for 45 minutes. Cell membranes (20 μg) are incubated in assay buffer containing 2 mg/ml fatty acid free bovine serum albumin (BSA), 20 μM GDP, and 0.1 nM [³⁵S]-γ-GTP (New England Nuclear). The assay buffer contains 50 mM Tris, 10 mM MgCl₂, and 100 mM NaCl, at pH 7.4. Incubation times are for 90 minutes at 30° C. The reaction is terminated by the addition of 4 mL of ice-cold wash buffer (50 mM Tris, 1 mg/mL BSA, pH 7.4) followed by rapid filtration under vacuum through GF/B glass fibre filters using a 12-tube Brandel cell harvester. The filters are washed 3 times with 4 mL of wash buffer. The filters are then dried, placed in scintillation fluid, and bound radioactivity is determined by liquid scintillation counting and reported in units of per minute (cpm). The [³⁵S]-γ-GTP binding of is determined (a) in the presence of 20 μM GDP (“total binding” (TB)), and (b) in the presence of 10 μM [³⁵S]-γ-GTP (“non-specific binding” (NSB)). The level of [³⁵S]-γ-GTP binding is reported as a percentage change with respect to basal levels. The “specific binding” (SB) of [³⁵S]-γ-GTP to the receptor is defined as the total binding less the non-specific binding (SB=TB−NSB), and this value is taken as 100%.

Method E. CB-1 GTPγ F³⁵S Binding Assay using cell membranes. Membranes are prepared from CHO-K1 cells stably transfected with the human CB-1 receptor cDNA. Membranes are prepared from cells as described by Bass et al, in “Identification and characterization of novel somatostatin antagonists,” Molecular Pharmacology. 50:709-715 (1996). GTPγ [35S] binding assays were performed in a 96 well FlashPlate™ format in duplicate using 100 μM GTPy[35S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix is then incubated with increasing concentrations of antagonist (10⁻¹ M to 10⁻⁵ M) for 10 minutes and challenged with the cannabinoid agonist 5-(1,1-dimethylheptyl)-2-[5-hydroxy-2-(3-hydroxypropyl)cyclohexyl]-phenol (10 μM). Assays are performed at 30° C. for one hour. The FlashPlates™ are then centrifuged at 2000 times G for 10 minutes. Stimulation of GTPy[35S] binding is then quantified using a Wallac Microbeta.EC50 calculations done using Prism™ by Graphpad. Inverse agonism is measured in the absence of agonist.

CB-1 FLIPR-based Functional Assay Protocol. CHO-K1 cells co-transfected with the human CB-1 receptor cDNA (obtained from Dr. Debra Kendall, University of Connecticut) and the promiscuous G-protein G16 were used for this assay. Cells were plated 48 hours in advance at 12,500 cells per well on collagen coated 384 well black clear assay plates. Cells were incubated for one hour with 4 μM Fluo-4 AM (Molecular Probes) in DMEM (Gibco) containing 2.5 mM probenicid and pluronic acid (0.04%). The plates were then washed 3 times with HEPES-buffered saline (containing probenicid; 2.5 mM) to remove excess dye. After 20 min the plates were added to the FLIPR individually and fluorescence levels was continuously monitored over an 80 second period. Compound additions were made simultaneously to all 384 wells after 20 seconds of baseline. Assays were performed in triplicate and 6 point concentration-response curves generated. Antagonist compounds were subsequently challenged with 3 μM WIN 55, 212-2 (agonist). Data were analyzed using Graph Pad Prism.

Detection of Inverse Agonists. The following cyclic-AMP assay protocol using intact cells was used to determine inverse agonist activity. Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C. Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01 N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

Cannabinoid agonists such as Δ9-tetrahydrocannabinol (Δ9-THC) and 5-(1,1-dimethylheptyl)-2-[5-hydroxy-2-(3-hydroxypropyl)cyclohexyl]-phenol have been shown to affect four characteristic behaviors in mice, collectively known as the Tetrad. For a description of these behaviors see: Smith, P. B., et al. in “The pharmacological activity of anandamide, a putative endogenous cannabinoid, in mice.” J. Pharmacol. Exp. Ther. 270(1), 219-227 (1994) and Wiley, J., et al. in “Discriminative stimulus effects of anandamide in rats,” Eur. J. Pharmacol., 276(1-2), 49-54 (1995). Reversal of these activities in the Locomotor Activity, Catalepsy, Hypothermia, and Hot Plate assays described below provides a screen for in vivo activity of CB-1 antagonists. All data is presented as % reversal from agonist alone using the following formula: (5-(1,1-dimethylheptyl)-2-[5-hydroxy-2-(3-hydroxypropyl)cyclohexyl]-phenol/agonist-vehicle/agonist)/(vehicle/vehicle-vehicle/agonist). Negative numbers indicate a potentiation of the agonist activity or non-antagonist activity. Positive numbers indicate a reversal of activity for that particular test.

Cannabinoid Receptor-2 (CB2) Functional Activity Assay.

Method A. Competition with [³H]WIN 55212-2. The functional activity of the compounds described herein at the CB2 receptor is determined by the ability of test compounds to displace [³H]WIN 55212-2 from the receptor in membranes derived from hCB2 stably transfected CHO cells (Perkin Elmer/NEN). Membranes are diluted in ice-cold assay buffer (10 mM HEPES, 100 mM NaCl, 10 mM MgCl₂, 2 μM GDP, 0.2% FAF-BSA, 10 μg/mL saponin) to 38 μg membranes/mL. Dilute ³⁵S-GTPγS to 1 nM in assay buffer. A positive agonist control with 20 μL of [³H]WIN 55212-2 is performed from 3 nM to 300 μM, with 20 μL of ³⁵S-GTPγS and 50 μL of membranes. A single-dose negative control with 20 μL of 10 μM Example 38J (IC50>10 μM), with 20 μL of ³⁵S-GTPγS and 50 μL of membranes is performed. The compounds described herein (20 μL of 1 nM to 10 μM) are tested against 20 μL of [³H]WIN 55212-2 at 30 μM, with 20 μL of ³⁵S-GTPγS and 50 μL of membranes. These solutions are incubated for 30 minutes at 30° C. The filters are washed 6 times with 10 mM sodium phosphate buffer (pH 7.4) at ambient temperature. The filters are counted using a liquid scintillation counter on the ³⁵S channel. Each compound/concentration is run in triplicate. A positive antagonism control is run for AM630 at the same concentrations as test compounds under the same conditions. The results for several examples described herein are shown in FIGS. 2a-2f.

Method B. Measurement ofadenylyl cyclase production. Although binding studies measure the affinity of a ligand for the receptor, such studies do not indicate the functional characteristics of the ligand (that is, whether it acts as an agonist, neutral antagonist, inverse agonist, etc.). Thus, many cannabinoid receptor ligands may also be conveniently classified according to their functional characteristics, for example, their effect upon cannabinoid receptor activity, for example, as an agonist, neutral antagonist, inverse agonist, etc. The CB2 receptor (GPCR) super-family is coupled to inhibition of adenylyl cyclase and activation of signal-regulated cascade (ERK).

The traditional model of G protein-coupled receptor (GPCR) action is based on the premise that the binding of an agonist to the receptor is necessary for receptor activation. However, it is now clear that some receptor activation occurs spontaneously, without agonist binding, the receptors being constitutively active. Cannabinoid CB2 receptors appear to be constitutively active. A large body of evidence for this has been obtained from high expression recombinant cell lines where cannabinoid receptor inverse agonists stimulate adenylyl cyclase and inhibit ERK (see, e.g., Bouboula et; Bouboula et 1997; Bouboula et 1999).

By sequestration of Gi proteins, cannabinoid inverse agonists not only inhibit constitutively active CB2 receptors but also inhibit receptor activation by other unrelated Gi-dependent receptors. In general, ligands that do not bind directly to a receptor, but do affect the receptor's function, may be described are numerous examples of so-called allosteric modulators of G-protein coupled receptors that bind to a site closely related to the receptor and modulate the function of the receptor (see, e.g., Vaulquelin et 2002).

Such sites may exist for the cannabinoid receptors; however, none have yet been identified. Thus, many cannabinoid receptor ligands may be further classified as: (a) cannabinoid receptor agonists, which activate the receptor; partial agonists also activate the receptor, but with lower efficacy than full agonist; (b) cannabinoid receptor inverse agonists, which both block the action of the agonist and attenuate receptor-constitutive activity; (c) cannabinoid receptor neutral antagonists, which block the action of the agonist but are ineffective on the receptor-constitutive activity; they may also be low efficacy partial agonists that behave

The functional activation of receptor is based on recombinant human receptor expressed in CHO cells (Felder et al., Mol. Pharmacol. 48:443-450, 1995). To determine the agonist activity or inverse agonist activity of any test compound, 50 μL of CB2-CHO cell suspension are mixed with test compound and 70 μL assay buffer containing 0.34 mM 3-isobutyl-1-methylxanthine and 5.1 μM of forskolin in 96-well plates. The assay buffer is comprised of Earle's Balanced Salt Solution supplemented with 5 mM MgCl2, 1 mM glutamin, 10 mM HEPES, and 1 mg/mL bovine serum albumin.

The mixture is incubated at room temperature for 30 minutes, and terminated by adding 30 μL/well of 0.5M HCl. The total intracellular cAMP level is quantified using the New England Nuclear Flashplate and cAMP radioimmunoassay kit. To determine the antagonist activity of test compound, the reaction mixture also contains 0.5 nM of the agonist CP55940, and the reversal of the CP55940 effect is quantified. Alternatively, a series of dose response curves for CP55940 is performed with increasing concentration of the test compound in each of the dose response curves.

Method C. Cyclic AMP Assay. Cannabinoid receptors CB1 and CB2 are coupled to inhibition of adenylyl cyclase (see, e.g., Bidault-Russell et 1990; et 1996). Adenylyl cyclase is an enzyme that catalyses the production of cyclic adenosine monophosphate (AMP). Thus, activation of the receptor leads to the inhibition of the production of cyclic AMP. Certain compounds, such as forskolin, stimulate adenylyl cyclase. Accumulation of cyclic AMP is then measured using a and is indicative of adenylyl cyclase activation. The amount of radioactivity can be measured and will be proportional to the level of cyclic AMP that is produced. The cyclic AMP assay is performed with a phosphodiesterase inhibitor present. This is necessary because phosphodiesterase is an enzyme that rapidly breaks down cyclic AMP. An example of a phosphodiesterase inhibitor is rolipram. The cyclic AMP assay is performed using cells that contain CB2 receptors only (Chinese Hamster Ovary Cells or Human Embryonic Kidney Cells).

The cells or tissues are incubated for 30 minutes at 37° C. with the cannabinoid receptor ligand and the phosphodiesterase inhibitor rolipram (Sigma) (50 μM) in phosphate buffered saline (PBS) containing 1 mg/ml bovine serum albumin (Sigma). The cells or tissues are then incubated for a further 30 minutes incubation with 1 μM forskolin (Sigma). The reaction is terminated by addition 0.1 M hydrochloric acid and the mixture is centrifuged in a to remove cell debris. The resulting pellet contains cell debris and the supernatant contains the [³H]cyclic AMP. A sample of a supernatant is removed and the pH is adjusted to pH using 1 M The cyclic AMP content is then measured using a radioimmunoassay kit ([³H] Biotrack assay TRK432, from Amersham Biosciences), following the manufacturers instructions. The amount of radioactivity in each sample is counted using a Beckman scintillation counter. The amount is cyclic AMP in each sample is calculated from the level of radioactivity.

Method D. [³⁵S]-γ-GTP Assay using cell homogenates. Activation of a G-protein coupled receptor by an agonist leads to the replacement of guanosine diphosphate (GDP) with guanosine triphosphate (GTP). The level of binding of GTP to the receptor is proportional to the level of receptor activation. The level of binding is measured by using a radiolabelled form of GTP called [³⁵S]-γ-GTP. Thus the radioactivity can be measured and is proportional to the amount of GTP bound to the receptor. The [³⁵S]-γ-GTP binding assay is performed with cells that contain CB1 receptors only or cells that contain CB2 receptors only (Chinese Hamster Ovary cells or human embryonic kidney cells, respectively). The [³⁵S]-γ-GTP binding assay may also be performed with tissues that contain CB1 receptors (e.g., brain) or CB2 receptors (e.g., spleen).

Cells that contain CB2 receptors only are removed from flasks by scraping, and are re-suspended in homogenisation buffer (0.32 M sucrose/50 mM Tris), and using an Ultra-Turrex. If tissues are used, the homogenate is prepared as for a radioligand binding assay, as described herein. The homogenate is diluted with Tris buffer (50 mM, pH 7.4) and centrifuged at 50,000 times g for 45 minutes. Cell membranes (20 μg) are incubated in assay buffer containing 2 mg/ml fatty acid free bovine serum albumin (BSA), 20 μM GDP, and 0.1 nM [³⁵S]-γ-GTP (New England Nuclear). The assay buffer contains 50 mM Tris, 10 mM MgCl₂, and 100 mM NaCl, at pH 7.4. Incubation times are for 90 minutes at 30° C. The reaction is terminated by the addition of 4 mL of ice-cold wash buffer (50 mM Tris, 1 mg/mL BSA, pH 7.4) followed by rapid filtration under vacuum through GF/B glass fibre filters using a 12-tube Brandel cell harvester. The filters are washed 3 times with 4 mL of wash buffer. The filters are then dried, placed in scintillation fluid, and bound radioactivity is determined by liquid scintillation counting and reported in units of per minute (cpm). The [³⁵S]-γ-GTP binding of is determined (a) in the presence of 20 μM GDP (“total binding” (TB)), and (b) in the presence of 10 μM [³⁵S]-γ-GTP (“non-specific binding” (NSB)). The level of [³⁵S]-γ-GTP binding is reported as a percentage change with respect to basal levels. The “specific binding” (SB) of [³⁵S]-γ-GTP to the receptor is defined as the total binding less the non-specific binding (SB=TB−NSB), and this value is taken as 100%.

Method E. CB-2 GTPγ F³⁵S Binding Assay. Membranes are prepared from CHO-K1 cells stably transfected with the human CB-2 receptor cDNA. Membranes are prepared from cells as described by Bass et al, in “Identification and characterization of novel somatostatin antagonists,” Molecular Pharmacology. 50:709-715 (1996). GTPγ [35S] binding assays are performed in a 96 well FlashPlate™ format in duplicate using 100 μM GTPγ[35S] and 10 μg membrane per well in assay buffer composed of 50 mM Tris HCl, pH 7.4, 3 mM MgCl₂, pH 7.4, 10 mM MgCl₂, 20 mM EGTA, 100 mM NaCl, 30 μM GDP, 0.1% bovine serum albumin and the following protease inhibitors: 100 μg/ml bacitracin, 100 μg/ml benzamidine, 5 μg/ml aprotinin, 5 μg/ml leupeptin. The assay mix is then incubated with increasing concentrations of antagonist (10¹ M to 10⁻⁵ M) for 10 minutes and challenged with the cannabinoid agonist 5-(1,1-dimethylheptyl)-2-[5-hydroxy-2-(3-hydroxypropyl)cyclohexyl]-phenol (10 μM). Assays are performed at 30° C. for one hour. The FlashPlates™ is then centrifuged at 2000 times G for 10 minutes. Stimulation of GTPy[35S] binding is then quantified using a Wallac Microbeta.EC50 calculations done using Prism™ by Graphpad. Inverse agonism was measured in the absence of agonist.

Detection of Inverse Agonists. The following cyclic-AMP assay protocol using intact cells was used to determine inverse agonist activity. Cells were plated into a 96-well plate at a plating density of 10,000-14,000 cells per well at a concentration of 100 μl per well. The plates were incubated for 24 hours in a 37° C. incubator. The media was removed and media lacking serum (100 μl) was added. The plates were then incubated for 18 hours at 37° C. Serum free medium containing 1 mM IBMX was added to each well followed by 10 μl of test compound (1:10 stock solution (25 mM compound in DMSO) into 50% DMSO/PBS) diluted 10× in PBS with 0.1% BSA. After incubating for 20 minutes at 37° C., 2 μM of Forskolin was added and then incubated for an additional 20 minutes at 37° C. The media was removed, 100 μl of 0.01 N HCl was added and then incubated for 20 minutes at room temperature. Cell lysate (75 μl) along with 25 μl of assay buffer (supplied in FlashPlate™ cAMP assay kit available from NEN Life Science Products Boston, Mass.) into a Flashplate. cAMP standards and cAMP tracer were added following the kit's protocol. The flashplate was then incubated for 18 hours at 4° C. The content of the wells were aspirated and counted in a Scintillation counter.

Cannabinoid agonists such as Δ9-tetrahydrocannabinol (Δ9-THC) and 5-(1,1-dimethylheptyl)-2-[5-hydroxy-2-(3-hydroxypropyl)cyclohexyl]-phenol have been shown to affect four characteristic behaviors in mice, collectively known as the Tetrad. For a description of these behaviors see: Smith, P. B., et al. in “The pharmacological activity of anandamide, a putative endogenous cannabinoid, in mice.” J. Pharmacol. Exp. Ther. 270(1), 219-227 (1994) and Wiley, J., et al. in “Discriminative stimulus effects of anandamide in rats,” Eur. J. Pharmacol., 276(1-2), 49-54 (1995). Reversal of these activities in the Locomotor Activity, Catalepsy, Hypothermia, and Hot Plate assays described below provides a screen for in vivo activity of CB-1 antagonists. All data is presented as % reversal from agonist alone using the following formula: (5-(1,1-dimethylheptyl)-2-[5-hydroxy-2-(3-hydroxypropyl)cyclohexyl]-phenol/agonist-vehicle/agonist)/(vehicle/vehicle-vehicle/agonist). Negative numbers indicate a potentiation of the agonist activity or non-antagonist activity. Positive numbers indicate a reversal of activity for that particular test.

Locomotor Activity. Male ICR mice (n=6; 17-19 g, Charles River Laboratories, Inc., Wilmington, Mass.) were pre-treated with test compound (sc, po, ip, or icv). Fifteen minutes later, the mice were challenged with 5-(1,1-dimethylheptyl)-2-[5-hydroxy-2-(3-hydroxypropyl)cyclohexyl]-phenol (sc). Twenty-five minutes after the agonist injection, the mice were placed in clear acrylic cages (431.8 cm×20.9 cm×20.3 cm) containing clean wood shavings. The subjects were allowed to explore surroundings for a total of about 5 minutes and the activity was recorded by infrared motion detectors (available from Coulbourn Instruments™, Allentown, Pa.) that were placed on top of the cages. The data was computer collected and expressed as “movement units.”

Catalepsy. Male ICR mice (n=6; 17-19 g upon arrival) were pre-treated with test compound (sc, po, ip or icv). Fifteen minutes later, the mice were challenged with 5-(1,1-dimethylheptyl)-2-[5-hydroxy-2-(3-hydroxypropyl)cyclohexyl]-phenol (sc). Ninety minutes post injection, the mice were placed on a 6.5 cm steel ring attached to a ring stand at a height of about 12 inches. The ring was mounted in a horizontal orientation and the mouse was suspended in the gap of the ring with fore- and hind-paws gripping the perimeter. The duration that the mouse remained completely motionless (except for respiratory movements) was recorded over a 3-minute period. The data were presented as a percent immobility rating. The rating was calculated by dividing the number of seconds the mouse remains motionless by the total time of the observation period and multiplying the result by 100. A percent reversal from the agonist was then calculated.

Hypothermia. Male ICR mice (n=5; 17-19 g upon arrival) were pretreated with test compounds (sc, po, ip or icv). Fifteen minutes later, mice were challenged with the cannabinoid agonist 5-(1,1-dimethyl-heptyl)-2-[5-hydroxy-2-(3-hydroxy-propyl)-cyclohexyl]-phenol (sc). Sixty-five minutes post agonist injection, rectal body temperatures were taken. This was done by inserting a small thermostat probe approximately 2-2.5 cm into the rectum. Temperatures were recorded to the nearest tenth of a degree

Hot Plate. Male ICR mice (n=7; 17-19 g upon arrival) are pre-treated with test compounds (sc, po, ip or iv). Fifteen minutes later, mice were challenged with a cannabinoid agonist 5-(1,1-dimethyl-heptyl)-2-[5-hydroxy-2-(3-hydroxy-propyl)-cyclohexyl]-phenol (sc). Forty-five minutes later, each mouse was tested for reversal of analgesia using a standard hot plate meter (Columbus Instruments). The hot plate was 10″×10″×0.75″ with a surrounding clear acrylic wall. Latency to kick, lick or flick hindpaw or jump from the platform was recorded to the nearest tenth of a second. The timer was experimenter activated and each test had a 40 second cut off. Data were presented as a percent reversal of the agonist induced analgesia.

Acute food intake studies in rats or mice: General Procedure. Adult rats or mice are used in these studies. After at least 2 days of acclimation to the vivarium conditions (controlled humidity and temperature, lights on for 12 hours out of 24 hours) food is removed from rodent cages. Experimental compounds or their vehicles are administered orally, intraperitoneally, subcutaneously or intravenously before the return of a known amount of food to cage. The optimal interval between dosing and food presentation is based on the half-life of the compound based on when brain concentrations of the compound is the highest. Food remaining is measured at several intervals.

Food intake is calculated as grams of food eaten per gram of body weight within each time interval and the appetite-suppressant effect of the compounds are compared to the effect of vehicle. In these experiments many strains of mouse or rat, and several standard rodent chows can be used.

Chronic weight reduction studies in rats or mice: General Procedure. Adult rats or mice are used in these studies. Upon or soon after weaning, rats or mice are made obese due to exclusive access to diets containing fat and sucrose in higher proportions than in the control diet. The rat strains commonly used include the Sprague Dawley bred through Charles River Laboratories. Although several mouse strains may be used, c57B1/6 mice are more prone to obesity and hyperinsulinemia than other strains. Common diets used to induce obesity include: Research Diets D12266B (32% fat) or D12451 (45% fat) and BioServ S3282 (60% fat). The rodents ingest chow until they are significantly heavier and have a higher proportion of body fat than control diet rats, often 9 weeks. The rodents receive injections (1 to 4 per day) or continuous infusions of experimental compounds or their vehicles either orally, intraperitoneally, subcutaneously or intravenously. Food intake and body weights are measured daily or more frequently. Food intake is calculated as grams of food eaten per gram of body weight within each time interval and the appetite-suppressant and weight loss effects of the compounds are compared to the effects of vehicle.

Food Intake. The following screen was used to evaluate the efficacy of test compounds for inhibiting food intake in Sprague-Dawley rats after an overnight fast. Male Sprague-Dawley rats were obtained from Charles River Laboratories, Inc. (Wilmington, Mass.). The rats were individually housed and fed powdered chow. They were maintained on a 12-hour light/dark cycle and received food and water ad libitum. The animals were acclimated to the vivarium for a period of one week before testing was conducted. Testing was completed during the light portion of the cycle. To conduct the food intake efficacy screen, rats were transferred to individual test cages without food the afternoon prior to testing, and the rats were fasted overnight. After the overnight fast, rats were dosed the following morning with vehicle or test compounds. A known antagonist was dosed (3 mg/kg) as a positive control, and a control group received vehicle alone (no compound). The test compounds were dosed at ranges between 0.1 and 100 mg/kg depending upon the compound. The standard vehicle was 0.5% (w/v) methylcellulose in water and the standard route of administration was oral. However, different vehicles and routes of administration were used to accommodate various compounds when required. Food was provided to the rats 30 minutes after dosing and the Oxymax automated food intake system (Columbus Instruments, Columbus, Ohio) was started. Individual rat food intake was recorded continuously at 10-minute intervals for a period of two hours. When required, food intake was recorded manually using an electronic scale; food was weighed every 30 minutes after food was provided up to four hours after food was provided. Compound efficacy was determined by comparing the food intake pattern of compound-treated rats to vehicle and the standard positive control.

Alcohol Intake. The following protocol evaluates the effects of alcohol intake in alcohol preferring (P) female rats (bred at Indiana University) with an extensive drinking history. The following references provide detailed descriptions of P rats: Li, T.-K., et al., “Indiana selection studies on alcohol related behaviors” in Development of Animal Models as Pharmacoqenetic Tools (eds McCleam C. E., Deitrich R. A. and Erwin V. G.), Research Monograph 6, 171-192 (1981) NIAAA, ADAMHA, Rockville, Md.; Lumeng, L, et al., “New strains of rats with alcohol preference and nonpreference” Alcohol And Aldehyde Metabolizing Systems. 3, Academic Press, New York, 537-544 (1977); and Lumeng, L, et al., “Different sensitivities to ethanol in alcohol-preferring and -nonpreferring rats,” Pharmacol. Biochem Behav. 16, 125-130 (1982). Female rats were given 2 hours of access to alcohol (10% v/v and water, 2-bottle choice) daily at the onset of the dark cycle. The rats were maintained on a reverse cycle to facilitate experimenter interactions. The animals were initially assigned to four groups equated for alcohol intakes: Group 1-vehicle (n=8); Group 2-positive control (e.g., 5.6 mg/kg AM251; n=8); Group 3-low dose test compound (n=8); and Group 4-high dose of test compound (n=8). Test compounds were generally mixed into a vehicle of 30% (w/v) β-cyclodextrin in distilled water at a volume of 1-2 ml/kg. Vehicle injections were given to all groups for the first two days of the experiment. This was followed by 2 days of drug injections (to the appropriate groups) and a final day of vehicle injections. On the drug injection days, drugs were given sc 30 minutes prior to a 2-hour alcohol access period. Alcohol intake for all animals was measured during the test period and a comparison was made between drug and vehicle-treated animals to determine effects of the compounds on alcohol drinking behavior. Additional drinking studies were done utilizing female C57BI/6 mice (Charles River). Several studies have shown that this strain of mice will readily consume alcohol with little to no manipulation required (Middaugh et al., “Ethanol Consumption by C57BL/6 Mice: Influence of Gender and Procedural Variables” Alcohol, 17 (3), 175-183, 1999; Le et al., “Alcohol Consumption by C57BL/6, BALA/c, and DBA/2 Mice in a Limited Access Paradigm” Pharmacology Biochemistry and Behavior, 47, 375-378, 1994). For our purposes, upon arrival (17-19 g) mice were individually housed and given unlimited access to powdered rat chow, water and a 10% (w/v) alcohol solution. After 2-3 weeks of unlimited access, water was restricted for 20 hours and alcohol was restricted to only 2 hours access daily. This was done in a manner that the access period was the last 2 hours of the dark part of the light cycle. Once drinking behavior stabilized, testing commenced. Mice were considered stable when the average alcohol consumption for 3 days was ±20% of the average for all 3 days. Day 1 of test consisted of all mice receiving vehicle injection (sc or ip). Thirty to 120 minutes post injection access was given to alcohol and water. Alcohol consumption for that day was calculated (g/kg) and groups were assigned (n=7-10) so that all groups had equivocal alcohol intake. On day 2 and 3, mice were injected with vehicle or drug and the same protocol as the previous day was followed. Day 4 was wash out and no injections were given. Data was analyzed using repeated measures ANOVA. Change in water or alcohol consumption was compared back to vehicle for each day of the test. Positive results would be interpreted as a compound that was able to significantly reduce alcohol consumption while having no effect on water

Oxygen Consumption. Whole body oxygen consumption is measured using an indirect calorimeter (Oxymax from Columbus Instruments, Columbus, Ohio) in male Sprague Dawley rats (if another rat strain or female rats are used, it will be specified). Rats (300-380 g body weight) are placed in the calorimeter chambers and the chambers are placed in activity monitors. These studies are done during the light cycle. Prior to the measurement of oxygen consumption, the rats are fed standard chow ad libitum. During the measurement of oxygen consumption, food is not available. Basal pre-dose oxygen consumption and ambulatory activity are measured every 10 minutes for 2.5 to 3 hours. At the end of the basal pre-dosing period, the chambers are opened and the animals are administered a single dose of compound (the usual dose range is 0.001 to 10 mg/kg) by oral gavage (or other route of administration as specified, i.e., sc, ip, iv). Drugs are prepared in methylcellulose, water or other specified vehicle (examples include PEG400, 30% beta-cyclo dextran and propylene glycol). Oxygen consumption and ambulatory activity are measured every 10 minutes for an additional 1-6 hours post-dosing. The Oxymax calorimeter software calculates the oxygen consumption (ml/kg/h) based on the flow rate of air through the chambers and difference in oxygen content at inlet and output ports. The activity monitors have 15 infrared light beams spaced one inch apart on each axis, ambulatory activity is recorded when two consecutive beams are broken and the results are recorded as counts. Resting oxygen consumption, during pre- and post-dosing, is calculated by averaging the 10-min O2 consumption values, excluding periods of high ambulatory activity (ambulatory activity count >100) and excluding the first 5 values of the pre-dose period and the first value from the post-dose period. Change in oxygen consumption is reported as percent and is calculated by dividing the post-dosing resting oxygen consumption by the pre-dose oxygen consumption *100. Experiments will typically be done with n=4-6 rats and results reported are mean+/−SEM.

An increase in oxygen consumption of >10% is considered a positive result. Historically, vehicle-treated rats have no change in oxygen consumption from pre-dose basal.

MTT J774 Murine Macrophage Assay. Cultures of the murine macrophage cell line J774 is an established model system with which to screen for activity of agents that influence osteoclast function as described by Luckman et al., “Heterocycle-containing bisphosphonates cause apoptosis and inhibit bone resorption by preventing protein: evidence from structure-activity relationships in J774,” Bone Miner. 13:1668-1678 (1998). MTT (3-(4,5-dimethylthiazol-2-yl)-2,5-diphenyl tetrazolium bromide) has an orange color and is soluble in cell culture media. The mitochondrial enzyme succinate dehydrogenase acts upon MTT in living cells to produce the insoluble purple colored formazan. The amount of formazan produced, as measured by spectroscopy, is proportional to the number of viable cells.

J774 cells are plated at 10⁴ cells per well in 150 μL αMEM in 96-well plates and grown overnight. The next day test compounds are added to the cultures, and cultures are continued for another hours. At the end of the culture period, cell survival is determined using the tetrazolium dye-based MTT assay as described by MacPherson et al., “Expression and functional role of nitric oxide synthase isoforms in human osteoblast-like cells,” Bone 24:179-185 (1999). MTT (5 mg/mL MTT in αMEM) is added to each well (1:10 v/v, 15 μL) and the cells incubated for 4 hours. The medium is carefully removed using a needle without dislodging the crystal layer. 100 μL acidified isopropanol (4 M HCl 1:100 v/v in isopropanol) is added to each well and the purple crystals allowed to dissolve. The absorbance is measured in a plate reader at 540 nm, with 690 nm as reference. The controls are a deep purple color, indicating a high number of live cells. The results for each test compound are expressed as a percentage of the average control value.

Test compounds are prepared as 100 mM solutions in DMSO, then diluted 100× in culture medium. From these 1 mM solutions, convenient quantities (3-15 μL) are added directly to the wells so as to give the desired final compound concentration. IC₅₀ values for each compound are calculated using GraphPad Prism (GraphPad Software, San Diego) and were defined as the concentration of agent required to reduce cell survival to 50% of the control value at 72 hours

Alamar Blue J774 Murine Macrophage Viability Assay. Alamar Blue J774 Murine J774 cells are plated at 10⁴ cells per well in 150 μL αMEM (a Modified Eagle Medium) in 96-well plates and grown overnight. The next day, compounds were added to the cultures, and culture was continued for another 72 hours. At the end of the culture period cell survival was determined using an Alamar Blue assay as described by Nociari et al., “A novel one-step, highly sensitive fluorimetric assay to evaluate cell-mediated cytotoxicity,” Journal of Immunological Methods, 213:157-167 (1998).

Alamar Blue is an oxidation-reduction sensitive indicator. The dye itself is in the oxidized state, which is blue and non-fluorescent. The dye can accept electrons from reducing species, such as NADPH and FADH, to form a reduced dye species, which is red and fluorescent, and the transformation from oxidized form to reduced form can be measured by fluorimetric or colorimetric means. For fluorescence measurements, 530-560 nm excitation and 590 nm emission wavelengths may be used. For measurements, absorbance is measured at 570 nm (reduced form) and 600 nm (oxidized form) followed by a calculation to determine the relative quantities of the two species. A high ratio of the reducing species, NADPH and FADH, to the corresponding oxidized species, NADP and FAD, is an indicator that cells are proliferating and viable. A low ratio indicates cells that are quiescent or non-viable.

Alamar Blue (Biosource International) is added undiluted to each well (1:10 v/v, 15 μL), the plate is incubated at 37° C. for 3-4 hours, and the fluorescence is measured at 570 nm, with a 25 nm bandwidth. A high reading indicate cells with normal viability, and a low reading indicates cells that have been damaged and are no longer proliferating normally. The controls give a high fluorescence reading, indicating a high number of live, healthy cells. A potent test compound gives a low fluorescence reading. The average results for each test compound, such as n=5, is expressed as a percentage of the average control value.

In general, compounds studied are made up as 100 mM solutions in DMSO, which are then diluted 100 or 1000× in culture medium (αMEM). From the resulting 1 mM or 100 μM solutions, convenient quantities (e.g. 3-15 μL) are added directly to the wells so as to give the desired final compound concentration. IC₅₀ values for individual agents are calculated using GraphPad Prism (GraphPad Software, San Diego) and are defined as the concentration of agent required to reduce cell survival to of the control value at 72 hours.

It is appreciated that this assay may offer advantages over other assays, including MTT assays, by permitting a higher throughput, higher sensitivity, the feature that it is non-damaging to the cells, and that it generally gives an identical result to MTT assay.

Rabbit Osteoclast Culture Assay. Osteoclast survival and activity are studied in cultures of rabbit osteoclasts. Osteoclasts are isolated from the long bones of 2-3 day-old rabbits as described by Coxon et al., “Protein geranylgeranylation is required for osteoclast formation, function, and survival: inhibition by bisphosphonates and GGTI-298,” J. Bone Miner. Res. 15:1467-1476 (2000), plated on dentine slices and cultured in αMEM supplemented with 10% FCS and penicillin and streptomycin at 37° C. in 5% CO₂ for 48 hours in the presence or absence of test compounds. At the end of the culture, the osteoclasts are identified by staining for tartrate-resistant acid phosphatase (TRAcP), and the resorption pit area is quantified by reflected light microscopy as described by van't H of et al., “Cytokine-induced nitric oxide inhibits bone resorption by inducing-apoptosis of osteoclast progenitors and J. Bone Miner. Res. 12:1797-1804 (1997).

Bone Marrow Assay Co-Culture Assay. Osteoclast formation and activity is studied using an adaptation of the osteoblast-bone marrow c-culture assay, as described by van't H of et al. (1997) and Takahashi et al., “Osteoblastic cells are involved in osteoclast formation,” Endocrinology 123:2600-2602 (1988) to screen for osteoclast-inhibitor and osteoclast-stimulatory agents.

Osteoblasts are isolated from the bones of 2-day-old mice by sequential collagenase digestion (type I collagenase, Sigma) and cultured in αMEM supplemented with 10% FCS and penicillin and streptomycin at 37° C. in 5% CO₂. Bone marrow cell populations containing osteoclast precursors are isolated from the long bones of 3-5 month old mice and erythrocytes are removed by Ficoll Hypaque density gradient centrifugation. The resulting bone marrow cells are washed with PBS and resuspended in culture medium. Osteoblasts and bone marrow cells are plated at 10⁴ cells/well and 2×10⁵ cells/well, respectively, in 96-well plates in 150 μL of αMEM supplemented with 10% FCS, antibiotics and 10 nM 1,25-dihydroxyvitamin D₃. Test substances are added on day 7 and the cultures are terminated on day 10. At the end of the culture period, osteoclasts are identified by TRAcP staining and resorption pits are quantified by reflected light microscopy.

Mouse Osteoblast Culture Assay. Osteoblasts are isolated as described above and plated at 10⁴ cells/well in 96-well plates in 100 μL of αMEM supplemented with 10% FCS and antibiotics. Test compounds are added after 24 hours and left for 72 hours. Cell viability is assessed using the Alamar Blue assay.

In Vivo Assay of C57/BL6 mice. Female 9 week-old C57/BL6 mice are housed in a designated animal facility and routinely maintained on a 12h:12h light/dark cycle and given ad libitum access to food and water.

Bilateral ovariectomy (Ovx) is performed on test animals under general anaesthesia. Sham ovariectomy (Sham) is similarly performed but with externalization and replacement of the ovaries. Animals are given a daily injection of (a) compound described herein (e.g. about a 6 mg/kg dose) in vehicle or (b) vehicle alone (e.g. corn oil). After 21 days, the animals are killed, and the tibial bones are dissected and used for bone mineral density measurements and analysis. Measurements of bone mineral density (BMD) at the left proximal tibial metaphysis are determined by peripheral quantitative computed tomography (pQCT) using an XCT Research M bone densitometer with voxel size of 70 μm and analysis software version 5.1.4. (Stratec Medizintechnik, Pforzheim, Germany). Daily quality assurance measurements are performed using a plexi-coated PVC-fluorinated hydrocarbon phantom.

Bone Histomorphometry is performed on left tibiae. The bones are dissected free of soft tissues, fixed in 4% buffered formalin/saline (pH 7.4) and embedded in methyl methacrylate. Longitudinal sections (4 μm) are then prepared and stained with Von Kossa and counterstained with Paragon. Histomorphometric measurements are made on sections of the proximal metaphysis distal to the epiphyseal growth plate at 20× magnification using a Zeiss Axioskop (Carl Zeiss, Welwyn Garden City, UK) coupled to an image analysis system. Bone histomorphometric variables are expressed according to the guidelines of the American Society of Bone and Mineral Research Nomenclature Committee (see, Eriksen, E. F., Axelrod, D. W., Melsen., F, 1994, Bone Raven Press, New York, USA).

Statistical-analyses are performed using SPSS for Windows version 9. Significant differences between groups are determined by ANOVA followed by post-hoc testing using Dunnet's post-test. All data are presented as means±SEM. Values of p less than 0.05 are considered significant.

Type I and Type IV Animal Models of Allergy.

Method A. Murine DNFB-induced allergic dermatitis. The model is produced by repeating antigen sensitization and induction in mouse to induce contact dermatitis that involves increase of IgE antibody titer, i.e., inflammation similar to atopic dermatitis, as described in J. Allergy Clin. Immunol. 100(6Pt2):39-44 (1997). Inflammation may occur via a delayed type allergic reaction due to T cells and the late phase allergic reaction due to mast cells. The weight of the spleen is measured simultaneously to examine systemic immunosuppressive action of a test compound.

An 0.15% (w/v) antigen solution of DNFB (2,4-dinitrofluorobenzene) is prepared in a mixed solution of acetone and olive oil (3:1, v/v), and 25 μL of the antigen solution is applied to each side of each ear of a 9-week-old female BALB/c mouse, once a week for 5 weeks.

The test compound is suspended in a 0.5% (w/v) methyl cellulose (MC) solution, in distilled water to prepare a 1 mg/mL suspension. By dilution, 0.1 mg/mL and 0.01 mg/mL suspensions are prepared. Positive controls, 0.5 mg/mL, 0.2 mg/mL and 0.1 mg/mL solutions of prednisolone (Sigma) are similarly prepared. Prednisolone is one of the adrenocorticosteroids that are suggested to be effective for the treatment of atopic dermatitis.

During the period from the next day of the third antigen application to the next day of the fifth antigen application, 10 mL/kg of the above-described test compound is administered once a day for 15 days. On the day of antigen application, the test compound is administered one hour before the antigen application, but administered 23 hours after the antigen application on the next day of the antigen application.

Before and 24 hours after the antigen application, the thickness of the ears is measured with a dial thickness gauge. The difference of thickness is used as an index of swelling, and compared with the positive controls. In addition, 24 hours after the fifth antigen application, the spleen is harvested from the mouse exsanguinated under anesthesia with ether. The wet weight of the spleen is measured.

Method B. Murine IgE-dependent allergic dermatitis. A model is produced by passive sensitization of a mouse with IgE and repeated antigen challenge to trigger triphase (early phase, late phase and very late phase) dermatitis, as described in Pharmacology, 60(2):97-104 (2000). The relation of mast cells and T cells, as well as invasion of eosinophils in local inflammatory sites is evaluated in this model, and reflect a part of the symptoms of atopic dermatitis.

A passive sensitization solution of Anti-DNP IgE (Yamasa Corp.) is prepared to 15 μg/mL with physiological saline, and 0.2 mL of the resulting solution is administered via the caudal vein to a 9 week-old female BALB/c mouse. A 0.15% (w/v) antigen solution of DNFB (2,4-dinitrofluorobenzene) is prepared in a mixed solution of acetone and olive oil (3:1, v/v), and 24 hours after the administration of the anti-DNP IgE, 25 μL of the antigen solution is applied to each side of each ear.

The test compound is suspended in an aqueous 0.5% MC solution in distilled water to prepare a 1 mg/mL suspension. In addition, as positive controls, 1 mg/mL ketotifen fumarate (Sigma) and 3 mg/mL pranlukast hydrate (extracted from Onon™ (Ono Pharmaceutical Co., Ltd.)) are prepared. Pranlukast hydrate is used as a leukotriene inhibitor for therapeutic agents for asthma and allergic rhinitis. Ketotifen fumarate is used as a chemical mediator release suppressor for asthma, allergic rhinitis, eczema, dermatitis, urticaria, dermal pruritis and allergic conjunctivitis.

From the antigen application day up to day 8 after the antigen application, 10 mL/kg of test compound is orally administered once a day for 9 days. To another group of mice, 10 mL/kg of test compound is orally administered once a day for 8 days from one day after the antigen application up to day 8 after the antigen application. To a third group of mice, 10 mL/kg of test compound is orally administered once a day for 7, 5 or 3 days in total from two, four or six days after the antigen application, and up to day 8 after the antigen application. During the period from the antigen application day to the start of the dosing of the test compound, and in place of the test compound, only an equal volume of the solvent is orally administered once a day. The test compound is administered one hour before the antigen application on the day of antigen application and one hour before the measurement of the thickness of the ear on day 8 after the antigen application.

Before the antigen application and one hour, 24 hours and 8 days after the antigen application, the thickness of the ear is measured with a dial thickness gauge. The difference between the values measured before the antigen application and at each time point was used as the index of swelling, and is compared with the positive controls.

Animal model of asthma. This model measures the effect of test compounds on antigen-induced immediate type asthma, late-type asthma, and airway hypersensitivity in guinea pigs. Animals are sensitized using an ultrasonic nebulizer (NE-U12; OMRON); 6-week old male Hartley guinea pig (Kudo, Co., Ltd.) continuously inhales 1% OVA (ovalbumin; Sigma)-containing physiological saline for 10 minutes per day for 8 consecutive days. Antigen challenge is performed one week after the last sensitization, 2% OVA is similarly inhaled for 5 minutes. 24 hours before and one hour after the OVA challenge, metyrapone-containing physiological saline (Aldrich, 10 mg/mL) is administered intravenously, and 30 minutes before the OVA induction, pyrilamine-containing physiological saline (Sigma, 10 mg/kg) is administered intraperitoneally.

Test compound is suspended in the aqueous 0.5% MC solution to obtain a solution of 60 mg/mL. The test compound is further diluted to 20, 6, and 2 mg/mL. As positive controls, 6 mg/mL pranlukast hydrate (extracted from Onon™ (Ono Pharmaceutical Co., Ltd.)) and 6 mg/mL prednisolone (Sigma) are similarly prepared.

During the 15-days from the start of sensitization to the antigen challenge, 5 mL/kg test compound is orally administered once a day. During the 8 days of sensitization, the test compound is given one hour before the sensitization. On the day of the antigen challenge, the test compound is given one hour before the challenge. As solvent controls, vehicle is similarly administered in accordance with OVA induction and physiological saline induction.

As positive controls, pranlukast hydrate is administered one hour before the challenge, while prednisolone is administered 16 hours and 2 hours before the challenge. The animals are fasted 16 to 18 hours before the oral administration.

Airway resistance is measured using a total respiratory function analysis system (Pulmos-I, M.I.P.S. Company). The pre value is measured, and subsequently, specific airway resistance (sRaw) per 100 breathes is measured one minute and 2, 4, 5, 6, 7 and 8 hours after the OVA challenge, and further once between 22 to 26 hours after the challenge, respectively. The average is used as sRaw at each measured time point. The increment ratio of sRaw is calculated by the following formula:

Increment ratio of sRaw (%)=[(sRaw at each measured time point−sRaw before the challenge)/(sRaw before the challenge)]×100.

Airway reactivity is measured 22 to 26 hours after the antigen challenge, for each of 0.0625, 0.125, 0.25, 0.5, 1 and 2 mg/mL solutions of physiological saline and acetylcholine (ACh) that are sequentially inhaled for one minute, until the sRaw is 2-fold or more to the baseline sRaw (sRaw after inhalation of physiological saline). Based on the ACh concentration and the concentration-resistance curve of sRaw, the ACh concentration required for the sRaw to achieve 100% increase from the baseline sRaw, i.e., PC100ACh is determined.

In vitro assay of leukotriene production from basophils. Leukotrienes (LTs) are known to be produced by basophils, mast cells, and other cells, and to be involved in the exacerbation of allergic disease, particularly allergic bronchial asthma. Test compound is prepared in dimethyl sulfoxide (DMSO) to a concentration of 0.01 mM, and then diluted with Tyrode solution (Sigma) to prepare 100 μM to 0.1 μM solutions (1% DMSO solution). For the cell assay, the solutions are further diluted to 10 μM to 0.01 μM (0.1% DMSO solution).

Using a syringe charged with 3.8% sodium citrate solution, 100 mL blood was obtained from human blood. Using 10×HBSS (−) (10× Hank's balanced salt solution, GIBCO), Percoll (Amersham) and Milli Q water, 1.070 g/mL, 1.079 g/mL and 1.088 g/mL Percoll-HBSS (−) is prepared and layered, and the obtained blood is loaded on the layers and centrifuged at 300 times g for 25 minutes. A cell fraction between the 1.070 g/mL Percoll-HBSS (−) layer and the 1.079 g/mL Percoll-HBSS (−) layer is recovered. Three volumes of HBSS (−) is added to the recovered cell suspension and centrifuged at 300 times g at 4° C. for 7 minutes. After centrifugation, the supernatant is discarded and the cells are rinsed once with HBSS (−) to recover a cell population of basophils.

The basophils are prepared to 2.5×10⁶ cells/mL with Tyrode solution, and 10 μg/mL recombinant human IL-3 (Genzyme/Techne) is added to a final concentration of 100 ng/mL. Immediately thereafter, 80 μL/well (2.5×10⁵ cells/well) of basophils are seeded on a round-bottom 96-well plate and incubated in 5% CO₂ at 37° C. for 30 minutes.

After preincubation, 10 μL/well of the above-described test compound is added and incubated in 5% CO₂ at 37° C. for 10 minutes. 10 μL/well Tyrode solution containing 1% DMSO is added as the solvent control group. 10 μL/well of anti-human IgE antibody diluted with Tyrode solution to 1, 3, 10, 30 and 100 μg/mL is added and incubated in 5% CO₂ at 37° C. for 30 minutes (final concentrations were 0.1, 0.3, 1, 3 and 10 μg/mL, respectively).

30 minutes after stimulation, the reaction mixtures are centrifuged at 3000 rpm at 4° C. for 5 minutes to recover 80 μL/well supernatants. The LT amount in the supernatants is assayed according to the manufacturer's protocol of LTs EIA kit (Amersham Pharmacia). The samples are diluted with Tyrode solution to 3-fold and 24-fold for the assay.

In vitro assay of leukotriene production from a rat mast cell line. Test compound is diluted and adjusted to 3, 1, 0.3 and 0.1 mM (100% DMSO solution), then diluted with E-MEM (EAGLE-MEM; Nikken Biomedical Lab.) to prepare solutions of 100 to 1 μM (1% DMSO solution). For the cell assay, the solutions are further diluted to 10 μM to 0.1 μM (0.1% DMSO solution).

1 mM PIPES (Dojin Molecular Technologies, Inc.), 14 mM NaCl, 0.5 mM KCl, 0.06 mM MgCl₂, 0.1 mM CaCl₂, 0.55 mM glucose and 0.1% BSA (bovine serum albumin; Sigma) are prepared with purified water and then adjusted to pH 7.4 using NaOH. 1 mg/mL anti-DNP IgE (monoclonal murine anti-DNP IgE; Yamasa Corp.) is diluted to 1000 fold with the PIPES buffer to prepare a 1 μg/mL solution. 10 mg/mL DNP-BSA is diluted to a concentration of 10 μg/mL with the PIPES buffer.

Culture medium: E-MEM containing heat-inactivated 10% FCS (fetal calf serum; Morgate Biotech), 100 units/mL penicillin and 100 μg/mL streptomycin (in the form of penicillin/streptomycin; GIBCO). After centrifuging and washing rat mast cell line RBL-2H3 (Human Science; 1×10⁶ cells/mL/tube) with the above-described culture medium, the cells are resuspended in the culture medium and cultured in a 75-cm² flask (Falcon 353136) for 3 days. After subculture, the cells are further cultured in a 225-cm² flask (Corning 431082) for 2 days. Semi-confluency (60 to 70% confluency) of the cells is confirmed and the cells are rinsed with HBSS and detached with trypsin-EDTA. The cells are recovered, washed by centrifugation with the culture medium, and resuspended in the culture medium. The concentration of the cells is adjusted to 2×10⁵ cells/mL and are seeded at 250 μl/well on a 96-well flat bottom culture plate (Falcon 3072) and cultured in 5% CO₂ at 37° C. for 20 hours.

The culture medium is discarded from the plate, the cells are rinsed with HBSS, 100 μL/well of 150 ng/mL anti-DNP IgE is added and incubated at 37° C. for 30 minutes for cell sensitization. The culture medium is discarded from the plate, the cells are rinsed with HBSS, 80 μL/well culture medium is added followed by 10 μL/well of test compound diluted with the culture medium to 1, 3, 10, 30 and 100 μM (the final concentrations are 0.1, 0.3, 1, 3 and 10 μM, respectively, in DMSO at a final concentration of 0.1%) and then incubated at 37° C. for 10 minutes.

10 μL/well DNP-BSA diluted with the culture medium to 150, 500, 1500 and 5000 ng/mL is added (final concentrations are 15, 50, 150 and 500 ng/mL, respectively) and incubated at 37° C. for 30 minutes. 30 minutes after the antigen stimulation, 20 μL/well supernatant is recovered and the LT amount is measured according to manufacturer's protocol of LTs EIA kit (Amersham Pharmacia).

Animal model of spontaneous scratching in NC mice. Itching is one of the main symptoms in the field of dermatology encompassing atopic dermatitis, urticaria and contact dermatitis. Currently, NC mouse is used as an animal model of atopic dermatitis. No dermatitis or scratching action is observed when the mouse is kept in an environment under the control of atmospheric microorganisms (in SPF environment). However, when the mouse is kept in a conventional environment, scratching action together with the onset of dermatitis can be observed from about week 8 and the symptom progresses into chronic symptom, as described in J. Dermatol. Sci. 25:20-28 (2001).

Test compound is suspended in MC dissolved in tap water (0.5% w/v) to prepare 1 mg/mL and 0.1 mg/mL suspensions. As positive controls, betamethasone valerate (Sigma) and tacrolimus hydrate (extracted from Prograf (Fujisawa Pharmaceutical Co., Ltd.)) are similarly prepared to 1 mg/mL. Betamethasone valerate is one of the adrenocorticosteroids that is considered to be effective for the treatment of atopic dermatitis. Tacrolimus hydrate is a therapeutic agent of atopic dermatitis which is described as an immunosuppressor.

Four week-old male NC/Jic mice (CLEA JAPAN, Inc.) are kept in the same cage for 12 days with mice (A) infected with rodent mite (Myoba musculi) that exhibit severe dermal lesions. The mice (A) are taken away from the cage and the NC/Jic mice are used at the age of 16 weeks. The mice are fed with solid feed CA-1 (CLEA JAPAN INC.) ad libitum and tap water as drinking water ad libitum, and kept at a temperature of 22±2° C. and a humidity of 55±10% under lighting from 8:00 a.m. to 20:00 p.m. From 10 days before the start of the experiment, the number of scratching movements with hind legs of the mice is visually counted (for 20 minutes; once daily) over 2 days or 3 days. Among the plural mice counted, mice with 50 or more average scratching movements per day are selected for use.

10 mL/kg test compound is orally administered once a day for 3 weeks. The behavior of the mice is observed (e.g. filmed) under unattended environment with a video camera to count the scratching motion with hind legs for one hour. Generally, the mice show several scratching motions for about one second. This series of scratching motions is counted as one scratching movement, and all such scratching movements are counted irrespective of scratched sites. The measurement is done on the starting day of administration, and 1, 3, 6, 10, 13, 17 and 20 days after the start of administration.

Additional Animal Models of Disease. The compounds described herein may be evaluated in a number of disease states known to be responsive to modulation of cannabinoid receptor function, including but not limited to:

(a) suppression of food intake and resultant weight loss in rats (Life Sciences, 63:113-117 (1998)); (b) reduction of sweet food intake in marmosets (Behavioural Pharm. 9:179 (1998)); (c) reduction of sucrose and ethanol intake in mice (Psychopharm. 132:104-106 (1997)); (d) increased motor activity and place conditioning in rats (Psychopharm. 135:324-332 (1998); Psychopharmacol 151:25-30 (2000)); (e) spontaneous locomotor activity in mice (J. Pharm. Exp. Ther. 277:586-594 (1996)); (f) reduction in opiate self-administration in mice (Sci. 283:401-404 (1999)); (g) bronchial in sheep and guinea pigs as models for the various phases of asthma (Abraham et al. “mediate antigen-induced late bronchial responses and prolonged airway hyper-responsiveness in sheep,” J. Clin. Invest. 93:776 (1993); Milne et al., “Role of VLA-4 integrin in leucocyte recruitment and bronchial in the guinea-pig,” Eur. J. Pharmacol. 282:243 (1995)); (h) mediation of the vasodilated state in advanced liver cirrhosis induced by carbon tetrachloride (Nature Medicine 7(7):827-832 (2001)); (i) amitriptyline-induced constipation in cynomologus monkeys is beneficial for the evaluation of laxatives (Biol. Pharm. Bulletin (Japan) 23(5)657-9 (2000)); 0) neuropathology of pediatric chronic intestinal pseudo-obstruction and animal models related to the neuropathology of pediatric chronic intestinal pseudo-obstruction (Journal of Pathology (England) 194(3)277-88 (2001)).

The disclosures of each of these literature citations are incorporated herein by reference for all they teach regarding the preparation, study, and evaluation of cells, tissues, animals, and animal models of disease.

### COMPOUND EXAMPLES

Each of the Examples prepared below exhibited an ¹H NMR spectrum consistent with the assigned structure. For selected Examples, additional compound characterization is described, including mass spectral analysis performed using FAB⁺ to observe the corresponding (M+H)⁺ parent ion.

### Example 1A

(4(S)-phenyloxazolidin-2-on-3-yl)acetyl chloride. A solution of 1.0 equivalent of (4(S)-phenyloxazolidin-2-on-3-yl)acetic acid (Evans, U.S. Pat. No. 4,665,171) and 1.3 equivalent of oxalyl chloride in 200 mL dichloromethane was treated with a catalytic amount of anhydrous dimethylformamide (85 μL/milliequivalent of acetic acid derivative) resulting in vigorous gas evolution. After 45 minutes all gas evolution had ceased and the reaction mixture was concentrated under reduced pressure to give an off-white solid after drying for 2 h under vacuum.

### Example 1B

(4(R)-phenyloxazolidin-2-on-3-yl)acetyl chloride. Prepared according to Example 1A, except that (4(R)-phenyloxazolidin-2-on-3-yl)acetic acid was used instead of (4(S)-phenyloxazolidin-2-on-3-yl)acetic acid (see, Evans & Sjogren, Tetrahedron Lett. 26:3783 (1985)).

### Example 1C

2-(4(S)-Phenyloxazolidin-2-on-3-yl)propanoyl chloride. A solution of 1 equivalent of Example 3A and 1.3 equivalent of oxalyl chloride in 200 mL CH₂Cl₂ (150 mL/g of propanoic acid derivative) was treated with a catalytic amount of anhydrous DMF (85 μL/mmole of propanoic acid derivative) resulting in vigorous gas evolution. After 45 min., all gas evolution had ceased and the reaction mixture was concentrated under reduced pressure to give an off-white solid after drying for 2 h. under vacuum.

### Example 2A

Methyl (4(S)-phenyloxazolidin-2-on-3-yl)acetate. A solution of (4(S)-phenyloxazolidin-2-on-3-yl)acetic acid (1 g, 4.52 mmol) (Evans in U.S. Pat. No. 4,665,171) in 20 mL of anhydrous methanol was treated hourly with 5 equivalents of acetyl chloride, for a total of 20 equivalents. The resulting solution was stirred overnight. The residue obtained after evaporation of the MeOH was redissolved in 30 mL of CH₂Cl₂ and treated with 50 mL of saturated aqueous Na₂CO₃. The organic layer was evaporated and dried (MgSO₄) to give a colorless oil (1.001 g, 94%); ¹H NMR (CDCl₃) δ 3.37 (d, J==18.0 Hz, 1H), 3.69 (s, 3H), 4.13 (t, J=8.3 Hz, 1H), 4.28 (d, J=18.0 Hz, 1H), 4.69 (t, J=8.8 Hz, 1H), 5.04 (t, J=8.4 Hz, 1H), 7.26-7.29 (m, 2H), 7.36-7.42 (m, 3H).

### Example 2B

Methyl 2-(4(S)-phenyloxazolidin-2-on-3-yl)propanoate. A solution of Example 2A (1 g, 4.25 mmol) in 10 mL of anhydrous THF at −78° C. was treated with 4.68 mL (4.68 mmol) of a 1 M solution of lithium bis(trimethylsilyl)amide in THF. The reaction mixture was stirred for 1 h. at about −70° C. before adding MeI (1.59 mL, 25.51 mmol). Upon complete conversion of the azetidinone, the reaction was quenched with saturated aqueous NH₄Cl and partitioned between EtOAc and water. The organic layer was washed sequentially with saturated aqueous sodium bisulfite, and saturated aqueous NaCl. The resulting organic layer was dried (MgSO₄) and evaporated to give a white solid (1.06 g, 93%, mixture of diasteromers); ¹H NMR (CDCl₃) δ 1.07/1.53 (d/d, J=7.5 Hz, 3H), 3.59/3.74 (s/s, 3H), 3.85/4.48 (q/q, J=7.5 Hz, 1H), 4.10-4.14 (m, 1H), 4.60-4.64/4.65-4.69 (m/m, 1H), 4.88-4.92/4.98-5.02 (m/m, 1H), 7.24-7.40 (m, 5H).

### Example 3A

2-(4(S)-Phenyloxazolidin-2-on-3-yl)propanoic acid. To a solution of Example 2B (1 g, 4.01 mmol) in 35 mL of MeOH was added, at 0° C., 14.3 mL (12.04 mmol) of a 0.84 M solution of LiOH in water. The reaction mixture was then stirred for 3 h. at ambient temperature. Upon complete hydrolysis of the azetidinone, the MeOH was removed by evaporation, the crude residue dissolved in CH₂Cl₂ and treated with saturated aqueous NaCl. The resulting organic layer was dried (MgSO₄) and evaporated to give a white solid (0.906 g, 96%, racemic mixture); ¹H NMR (CDCl₃) δ 1.13/1.57 (d/d, J=7.5 Hz, 3H), 3.75/4.50 (q/q, J=7.5 Hz, 1H), 4.10-4.16 (m, 1H), 4.62-4.72 (m, 1H), 4.92-5.03 (m, 1H), 7.32-7.43 (m, 5H).

### Example 4A

General procedure for amide formation from an activated ester derivative. N-Benzyloxycarbonyl-L-aspartic acid β-t-butyl ester β-(3-trifluoromethyl)benzylamide. A solution of N-benzyloxyvcarbonyl-L-aspartic acid β-t-butyl ester α-N-hydroxysuccinimide ester (1.95 g, 4.64 mmol, Advanced ChemTech) in 20 mL of dry tetrahydrofuran was treated with 0.68 mL (4.74 mmol) of 3-(trifluoromethyl)benzyl amine. Upon completion (TLC, 60:40 hexanes/ethyl acetate), the mixture was evaporated, and the resulting oil was partitioned between dichloromethane and a saturated aqueous solution of sodium bicarbonate. The organic laer was evaporated to give 2.23 g (quantitative yield) as a white solid; ¹H NMR (CDCl₃) δ 1.39 (s, 9H), 2.61 (dd, J=6.5 Hz, J=17.2 Hz, 1H), 2.98 (dd, J=3.7 Hz, J=17.0 Hz, 1H), 4.41 (dd, J=5.9 Hz, J=15.3 Hz, 1H), 4.50-4.57 (m, 2H), 5.15 (s, 2H), 5.96-5.99 (m, 1H), 6.95 (s, 1H), 7.29-7.34 (m, 5H), 7.39-7.43 (m, 2H), 7.48-7.52 (m, 2H).

### Examples 4

were prepared according to the procedure of Example 4A, except that N-benzyloxycarbonyl-L-aspartic acid β-t-butyl ester α-N-hydroxysuccinimide ester was replaced by the appropriate amino acid derivative, and 3-(trifluoromethyl)benzyl amine was replaced with the appropriate amine.

### Example 4B

N-Benzyloxycarbonyl-L-aspartic acid β-t-butyl ester α-[4-(2-phenylethyl)]piperazinamide. N-benzyloxycarbonyl-L-aspartic acid β-t-butyl ester α-N-hydroxysuccinimide ester (5.0 g, 12 mmol, Advanced ChemTech) and 4-(phenylethyl)piperazine 2.27 mL (11.9 mmol) gave 5.89 g (quantitative yield) as an off-white oil; ¹H NMR (CDCl₃) δ 1.40 (s, 9H), 2.45-2.80 (m, 10H), 3.50-3.80 (m, 4H), 4.87-4.91 (m, 1H), 5.08 (s, 2H), 5.62-5.66 (m, 1H), 7.17-7.33 (m, 10H).

### Example 4C

N-Benzyloxycarbonyl-L-glutamic acid γ-t-butyl ester α-(3-trifluoromethyl)benzylamide. N-benzyloxycarbonyl-L-glutamic acid β-t-butyl ester α-N-hydroxysuccinimide ester (4.83 g, 11.1 mmol, Advanced ChemTech) and 3-(trifluoromethyl)benzylamine) 1.63 mL (11.4 mmol) gave 5.41 g (98%) as an off-white solid; ¹H NMR (CDCl₃) δ 1.40 (s, 9H), 1.88-1.99 (m, 1H), 2.03-2.13 (m, 1H), 2.23-2.33 (m, 1H), 2.38-2.47 (m, 1H), 4.19-4.25 (s, 1H), 4.46-4.48 (m, 2H), 5.05-5.08 (m, 2H), 5.67-5.72 (m, 1H), 7.27-7.34 (m, 5H), 7.39-7.43 (m, 2H), 7.48-7.52 (m, 2H).

### Example 4D

N-Benzyloxycarbonyl-L-glutamic acid γ-t-butyl ester α-[4-(2-phenylethyl)]piperazinamide. N-benzyloxycarbonyl-L-glutamic acid γ-t-butyl ester α-N-hydroxysuccinimide ester (5.0 g, 12 mmol, Advanced ChemTech) and 4-(phenylethyl)piperazine 2.19 mL (11.5 mmol) gave 5.87 g (quantitative yield) as an off-white oil; ¹H NMR (CDCl₃) δ 1.43 (s, 9H); 1.64-1.73 (m, 1H); 1.93-2.01 (m, 1H); 2.23-2.40 (m, 2H); 2.42-2.68 (m, 6H); 2.75-2.85 (m, 2H); 3.61-3.74 (m, 4H); 4.66-4.73 (m, 1H); 5.03-5.12 (m, 2H); 5.69-5.72 (m, 1H); 7.16-7.34 (m, 10H).

### Example 4E

N-Benzyloxycarbonyl-L-aspartic acid β-t-butyl ester α-[4-(2-phenylethyl)]piperazinamide. N-benzyloxycarbonyl-L-aspartic acid β-t-butyl ester α-N-hydroxysuccinimide ester (5.0 g, 12 mmol, Advanced ChemTech) and 4-(phenylethyl)piperazine 2.27 mL (11.9 mmol) gave 5.89 g (quantitative yield) as an off-white oil; ¹H NMR (CDCl₃) δ 1.40 (s, 9H), 2.45-2.80 (m, 10H), 3.50-3.80 (m, 4H), 4.87-4.91 (m, 1H), 5.08 (s, 2H), 5.62-5.66 (m, 1H), 7.17-7.33 (m, 10H).

### Example 4F

N-Benzyloxycarbonyl-L-glutamic acid γ-t-butyl ester α-(3-trifluoromethyl)benzylamide. N-benzyloxycarbonyl-L-glutamic acid γ-t-butyl ester α-N-hydroxysuccinimide ester (4.83 g, 11.1 mmol, Advanced ChemTech) and 3-(trifluoromethyl)benzylamine) 1.63 mL (11.4 mmol) gave 5.41 g (98%) as an off-white solid; ¹H NMR (CDCl₃) δ 1.40 (s, 9H), 1.88-1.99 (m, 1H), 2.03-2.13 (m, 1H), 2.23-2.33 (m, 1H), 2.38-2.47 (m, 1H), 4.19-4.25 (s, 1H), 4.46-4.48 (m, 2H), 5.05-5.08 (m, 2H), 5.67-5.72 (m, 1H), 7.27-7.34 (m, 5H), 7.39-7.43 (m, 2H), 7.48-7.52 (m, 2H).

### Example 4G

N-Benzyloxycarbonyl-L-glutamic acid γ-t-butyl ester α-[4-(2-phenylethyl)]piperazinamide. N-benzyloxycarbonyl-L-glutamic acid γ-t-butyl ester α-N-hydroxysuccinimide ester (5.0 g, 12 mmol, Advanced ChemTech) and 4-(phenylethyl)piperazine 2.19 mL (11.5 mmol) gave 5.87 g (quantitative yield) as an off-white oil; ¹H NMR (CDCl₃) δ 1.43 (s, 9H); 1.64-1.73 (m, 1H); 1.93-2.01 (m, 1H); 2.23-2.40 (m, 2H); 2.42-2.68 (m, 6H); 2.75-2.85 (m, 2H); 3.61-3.74 (m, 4H); 4.66-4.73 (m, 1H); 5.03-5.12 (m, 2H); 5.69-5.72 (m, 1H); 7.16-7.34 (m, 10H).

### Example 6A

General procedure for amide formation from a carboxylic acid. Illustrated for N-Benzyloxycarbonyl-D-aspartic acid β-t-butyl ester α-(3-trifluoromethyl)benzylamide. A solution of 1 g (2.93 mmol) of N-benzyloxycarbonyl-D-aspartic acid β-t-butyl ester monohydrate (Novabiochem) in 3-4 mL of dichloromethane was treated by sequential addition of 0.46 mL (3.21 mmol) of 3-(trifluoromethyl)benzylamine, 0.44 g (3.23 mmol) of 1-hydroxy-7-benzotriazole, and 0.62 g (3.23 mmol) of 1-[3-(dimethylamino)propyl]-3-ethylcarbodiimide hydrochloride. After at least 12 hours at ambient temperature or until complete as determined by thin layer chromatography (95:5 dichloromethane/methanol eluent), the reaction mixture was washed sequentially with a saturated aqueous sodium bicarbonate solution and with distilled water. The organic layer was evaporated to give 1.41 g (quantitative yield) as an off-white solid; ¹H NMR (CDCl₃) δ 1.39 (s, 9H); 2.61 (dd, J=6.5 Hz, J=17.2 Hz, 1H); 2.98 (dd, J=4.2 Hz, J=17.2 Hz, 1H); 4.41 (dd, J=5.9 Hz, J=15.3 Hz, 1H); 4.50-4.57 (m, 2H); 5.10 (s, 2H); 5.96-6.01 (m, 1H); 6.91-7.00 (m, 1H); 7.30-7.36 (m, 5H); 7.39-7.43 (m, 2H); 7.48-7.52 (m, 2H).

### Examples 6

Were prepared according to the procedure of Example 6A, except that N-benzyloxycarbonyl-D-aspartic acid β-t-butyl ester monohydrate was replaced by the appropriate amino acid derivative, and 3-(trifluoromethyl)benzyl amine was replaced with the appropriate amine.

### Example 6B

N-Benzyloxycarbonyl-D-glutamic acid γ-t-butyl ester α-(3-trifluoromethyl)benzylamide. N-benzyloxycarbonyl-D-glutamic acid γ-t-butyl ester (1.14 g, 3.37 mmol) and 0.53 mL (3.70 mmol, Novabiochem) of 3-(trifluoromethyl)benzylamine gave 1.67 g (quantitative yield) as an off-white solid.

### Example 6C

N-Benzyloxycarbonyl-L-glutamic acid α-t-butyl ester γ-(4-cyclohexyl)piperazinamide. N-benzyloxycarbonyl-L-glutamic acid α-t-butyl ester (1.36 g, 4.03 mmol) and 0.746 g (4.43 mmol) of 1-cyclohexylpiperazine gave 1.93 g (98%) as an off-white solid; ¹H NMR (CDCl₃) δ 1.02-1.12 (m, 5H); 1.43 (s, 9H), 1.60-1.64 (m, 1H); 1.80-1.93 (m, 5H); 2.18-2.52 (m, 8H); 3.38-3.60 (m, 4H); 4.20-4.24 (m, 1H); 5.03-5.13 (m, 2H); 5.53-5.57 (m, 1H); 7.28-7.34 (m, 5H).

### Example 6D

N-Benzyloxycarbonyl-D-aspartic acid β-t-butyl ester α-(2-fluoro-3-trifluoromethyl)benzylamide. N-benzyloxycarbonyl-D-aspartic acid β-t-butyl ester monohydrate (Novabiochem) (0.25 g, 0.73 mmol) and 0.12 mL of (2-fluoro-3-trifluoromethyl)benzylamine gave 0.365 g (quantitative yield) as an off-white solid; ¹H NMR (CDCl₃) δ 1.38 (s, 9H); 2.59 (dd, J=6.5 Hz, J=17.0 Hz, 1H); 2.95 (dd, J=4.3 Hz, J=17.0 Hz, 1H); 4.46-4.56 (m, 3H); 5.11 (s, 2H); 5.94-5.96 (m, 1H); 7.15 (t, J=8.0 Hz, 1H); 7.30-7.36 (m, 5H); 7.47-7.52 (m, 2H).

### Example 6E

N-Benzyloxycarbonyl-D-aspartic acid β-t-butyl ester α-[(S)-α-methylbenzyl]amide. N-benzyloxycarbonyl-D-aspartic acid β-t-butyl ester monohydrate (Novabiochem) (0.25 g, 0.73 mmol) and 0.094 mL of (S)-α-methylbenzylamine gave 0.281 g (90%) as an off-white solid; ¹H NMR (CDCl₃) δ 1.41 (s, 9H); 1.44 (d, J=7.0 Hz, 3H); 2.61 (dd, J=7.0 Hz, J=17.0 Hz, 1H); 2.93 (dd, J=4.0 Hz, J=17.5 Hz, 1H); 4.50-4.54 (m, 1H); 5.04-5.14 (m, 3H); 5.94-5.96 (m, 1H); 6.76-6.80 (m, 1H); 7.21-7.37 (m, 10H).

### Example 6F

N-Benzyloxycarbonyl-D-aspartic acid β-t-butyl ester α-[(R)-α-methylbenzyl]amide. N-benzyloxycarbonyl-D-aspartic acid β-t-butyl ester monohydrate (Novabiochem) (0.25 g, 0.73 mmol) and 0.094 mL of (R)-α-methylbenzylamine gave 0.281 g (90%) as an off-white solid; ¹H NMR (CDCl₃) δ 1.38 (s, 9H); 1.43 (d, J=6.9 Hz, 3H); 2.54 (dd, J=7.3 Hz, J=17.2 Hz, 1H); 2.87 (dd, J=4.1 Hz, J=17.3 Hz, 1H); 4.46-4.50 (m, 1H); 4.99-5.15 (m, 3H); 5.92-5.96 (m, 1H); 6.78-6.82 (m, 1H); 7.21-7.33 (m, 10H).

### Example 6G

N-Benzyloxycarbonyl-D-aspartic acid γ-t-butyl ester α-[N-methyl-N-(3-trifluoromethylbenzyl)]amide. N-benzyloxycarbonyl-D-aspartic acid γ-t-butyl ester (0.303 g, 0.89 mmol, Novabiochem) and 0.168 g (0.89 mmol,) of N-methyl-N-(3-trifluoromethylbenzyl)amine gave 0.287 g (65%) as an off-white solid; ¹H NMR (CDCl₃) δ 1.40 (s, 9H); 2.55 (dd, J=5.8 Hz, J=15.8 Hz, 1H); 2.81 (dd, J=7.8 Hz, J=15.8 Hz, 1H); 3.10 (s, 3H); 4.25 (d, J=15.0 Hz, 1H); 4.80 (d, J=15.5 Hz, 1H); 5.01-5.13 (m, 3H); 5.52-5.55 (m, 1H); 7.25-7.52 (m, 10H).

### Example 6H

N-Benzyloxycarbonyl-D-aspartic acid β-t-butyl ester α-[(S)-1-(3-trifluoromethylphenyl)ethyl]amide. N-benzyloxycarbonyl-D-aspartic acid β-t-butyl ester monohydrate (Novabiochem) (84 mg, 0.25 mmol) and 47 mg of (S)-1-(3-trifluoromethylphenyl)ethylamine gave 122 mg (quantitative yield) as an off-white solid.

### Example 6I

N-Benzyloxycarbonyl-D-aspartic acid β-t-butyl ester α-[(R)-1-(3-trifluoromethylphenyl)ethyl]amide. N-benzyloxycarbonyl-D-aspartic acid β-t-butyl ester monohydrate (Novabiochem) (150 mg, 0.44 mmol) and 83 mg of (R)-1-(3-trifluoromethylphenyl)ethylamine gave 217 mg (quantitative yield) as an off-white solid.

### Example 6J

N-Benzyloxycarbonyl-D-glutamic acid α-methyl ester γ-(3-trifluoromethyl)benzylamide. N-benzyloxycarbonyl-D-glutamic acid α-methyl ester (508 mg, 1.72 mmol) and 317 mg (1.81 mmol) of 3-(trifluoromethyl)benzylamine gave 662 mg (85%) as an off-white solid.

### Example 6K

N-tButyloxycarbonyl-(S)-(benzyl)-D-cysteine-[4-(2-(1-piperidyl)ethyl)]piperidinenamide. Prepared according to Example 6A, N-t-Butyloxycarbonyl-(S)-Benzyl- D-cysteine (0.289 g, 0.93 mmole) and 4-[2-(1-piperidyl)ethyl]piperidine (0.192 g, 0.98 mmole) were combined in dichloromethane (20 mL) to give 0.454 g (quantitative yield) as an off-white solid. ¹H NMR (CDCl₃) δ 0.89-1.15 (m, 2H); 1.39-1.44 (m, 16H); 1.54-1.61 (m, 4H); 1.62-1.71 (m, 1H); 2.21-2.35 (m, 5H); 2.49-2.58 (m, 2H); 2.66-2.74 (m, 1H); 2.79-2.97 (m, 1H); 3.67-3.76 (m, 3H); 4.48-4.51 (m, 1H); 4.72-4.75 (m, 1H); 5.41-5.44 (m, 1H); 7.19-7.34 (m, 5H).

### Example 7A

N-[(9H-Fluoren-9-yl)methoxycarbonyl]-O-(benzyl)-D-serine t-Butyl ester. N-[(9H-Fluoren-9-yl)methoxycarbonyl]-O-(benzyl)-D-serine (0.710 g, 1.70 mmole) in dichloromethane (8 mL) was treated with t-butyl acetate (3 mL) and concentrated sulfuric acid (40 μL) in a sealed flask at 0° C. Upon completion (TLC), the reaction was quenched with of dichloromethane (10 mL) and saturated aqueous potassium bicarbonate (15 mL). The organic layer was washed with distilled water, and evaporated. The resulting residue was purified by flash column:chromatography (98:2 dichloromethane/methanol) to yield 0.292 g (77%) as a colorless oil; ¹H NMR (CDCl₃) δ 1.44 (s, 9H); 3.68 (dd, J=2.9 Hz, J=9.3 Hz, 1H); 3.87 (dd, J=2.9 Hz, J=9.3 Hz, 1H); 4.22 (t, J=7.1 Hz, 1H); 4.30-4.60 (m, 5H); 5.64-5.67 (m, 1H); 7.25-7.39 (m, 9H); 7.58-7.61 (m, 2H); 7.73-7.76 (m, 2H).

### Example 8A

O-(Benzyl)-D-serine t-Butyl ester. Example 7A (0.620 g, 1.31 mmol) in dichloromethane (5 mL) was treated with tris(2-aminoethyl)amine (2.75 mL) for 5 h. The resulting mixture was washed twice with a phosphate buffer (pH=5.5), once with saturated aqueous potassium bicarbonate, and evaporated to give 0.329 g (quantitative yield) as an off-white solid; ¹H NMR (CD₃OD) δ 1.44 (s, 9H); 3.48 (dd, J=J′=4.2 Hz, 1H); 3.61 (dd, J=4.0 Hz, J=9.2 Hz, 1H); 3.72 (dd, J=4.6 Hz, J=9.2 Hz, 1H); 4.47 (d, J=12.0 Hz, 1H); 4.55 (d, J=12.0 Hz, 1H); 7.26-7.33 (m, 5H).

### Example 9A

General procedure for hydrogenation of a benzyloxycarbonyl amine. L-aspartic acid β-t-butyl ester α-(3-trifluoromethyl)benzylamide. A suspension of 2.23 g (4.64 mmol) of N-benzyloxycarbonyl-L-aspartic acid β-t-butyl ester α-(3-trifluoromethyl)benzylamide and palladium (5% wt. on activated carbon, 0.642 g) in 30 mL of methanol was held under an atmosphere of hydrogen until complete conversion as determined by thin layer chromatography (95:5 dichloromethane/methanol eluent). The reaction was filtered to remove the palladium over carbon and the filtrate was evaporated to give 1.52 g (96%) as an oil; ¹H NMR (CDCl₃) δ 1.42 (s, 9H); 2.26 (brs, 2H); 2.63-2.71 (m, 1H); 2.82-2.87 (m, 1H); 3.75-3.77 (m, 1H); 4.47-4.50 (m, 2H); 7.41-7.52 (m, 4H); 7.90 (brs, 1H).

### Examples 9

Were prepared according to the procedure of Example 9A, except that N-benzyloxycarbonyl-L-aspartic acid β-t-butyl ester α-(3-trifluoromethyl)benzylamide was replaced by the appropriate amino acid derivative.

### Example 9B

L-aspartic acid β-t-butyl ester α-[4-(2-phenylethyl)]piperazinamide. N-benzyloxycarbonyl-L-aspartic acid β-t-butyl ester α-[4-(2-phenylethyl)]piperazinamide (5.89 g, 11.9 mmol) gave 4.24 g (98%) as an off-white oil; ¹H NMR (CDCl₃): δ 1.42 (s, 9H); 2.61-2.95 (m, 10H); 3.60-3.90 (m, 4H); 4.35-4.45 (m, 1H); 7.17-7.29 (m, 5H).

### Example 9C

D-aspartic acid β-t-butyl ester α-(3-trifluoromethyl)benzylamide. N-benzyloxycarbonyl-D-aspartic acid β-t-butyl ester α-(3-trifluoromethyl)benzylamide (1.41 g, 2.93 mmol) gave 0.973 g (96%) as an off-white oil; ¹H NMR (CDCl₃): δ 1.42 (s, 9H); 2.21 (brs, 2H); 2.67 (dd, J=7.1 Hz, J=16.8 Hz, 1H); 2.84 (dd, J=3.6 Hz, J=16.7 Hz, 1H); 3.73-3.77 (m, 1H); 4.47-4.50 (m, 2H); 7.41-7.52 (m, 4H); 7.83-7.87 (m, 1H).

### Example 9D

L-glutamic acid γ-t-butyl ester α-(3-trifluoromethyl)benzylamide. N-benzyloxycarbonyl-L-glutamic acid γ-t-butyl ester α-(3-trifluoromethyl)benzylamide (5.41 g, 10.9 mmol) gave 3.94 g (quantitative yield) as an off-white oil; ¹H NMR (CDCl₃): δ 1.41 (s, 9H); 1.73-1.89 (m, 3H); 2.05-2.16 (m, 1H); 2.32-2.38 (m, 2H); 3.47 (dd, J=5.0 Hz, J=7.5 Hz, 1H); 4.47-4.49 (m, 2H); 7.36-7.54 (m, 4H); 7.69-7.77 (m, 1H).

### Example 9E

L-glutamic acid γ-t-butyl ester α-[4-(2-phenylethyl)]piperazinamide. N-benzyloxycarbonyl-L-glutamic acid γ-t-butyl ester α-[4-(2-phenylethyl)]piperazinamide (5.86 g, 11.50 mmol) gave 4.28 g (99%) as an off-white oil; ¹H NMR (CDCl₃) δ 1.39 (s, 9H); 2.00-2.08 (m, 1H); 2.38-2.46 (m, 1H); 2.55-2.90 (m, 9H); 3.61-3.82 (m, 4H); 4.48-4.56 (m, 1H); 7.17-7.26 (m, 5H).

### Example 9F

D-glutamic acid γ-t-butyl ester α-(3-trifluoromethyl)benzylamide. N-benzyloxycarbonyl-D-glutamic acid γ-t-butyl ester α-(3-trifluoromethyl)benzylamide (1.667 g, 3.37 mmol) gave 1.15 g (94%) as an off-white oil; ¹H NMR (CDCl₃) δ 1.41 (s, 9H); 1.80-2.20 (m, 4H); 2.31-2.40 (m, 2H); 3.51-3.59 (m, 1H); 4.47-4.49 (m, 2H); 7.39-7.52 (m, 4H); 7.71-7.79 (m, 1H).

### Example 9G

L-glutamic acid α-t-butyl ester γ-(4-cyclohexyl)piperazinamide. N-Benzyloxycarbonyl-L-glutamic acid α-t-butyl ester γ-(4-cyclohexyl)piperazinamide (1.93 g, 3.96 mmol) gave 1.30 g (93%) as an off-white oil; ¹H NMR (CDCl₃) δ 1.02-1.25 (m, 5H); 1.41 (s, 9H); 1.45-1.50 (m, 1H); 1.56-1.60 (m, 1H); 1.69-1.80 (m, 6H); 3.30 (dd, J=4.8 Hz, J=8.5 Hz, 1H); 3.44 (t, J=9.9 Hz, 2H); 3.56 (t, J=9.9 Hz, 2H).

### Example 9H

D-aspartic acid β-t-butyl ester α-(2-fluoro-3-trifluoromethyl)benzylamide. N-benzyloxycarbonyl-D-aspartic acid β-t-butyl ester α-(2-fluoro-3-trifluoromethyl)benzylamide (0.36 g, 0.72 mmol) gave 0.256 g (92%) as an off-white oil; ¹H NMR (CDCl₃) δ 1.39 (s, 9H); 2.50 (brs, 2H); 2.74 (dd, J=7.0 Hz, J=16.5 Hz, 1H); 2.86 (dd, J=4.8 Hz, J=16.8 Hz, 1H); 3.89 (brs, 2H); 4.47-4.57 (m, 2H); 7.16 (t, J=7.8 Hz, 1H); 7.48 (t, J=7.3 Hz, 1H); 7.56 (t, J=7.3 Hz, 1H); 7.97-8.02 (m, 1H).

### Example 9I

D-aspartic acid β-t-butyl ester α-[(S)-α-methyl]benzylamide. N-benzyloxycarbonyl-D-aspartic acid β-t-butyl ester α-[(S)-α-methylbenzyl]amide (0.275 g, 0.65 mmol) gave 0.17 g (90%) as an off-white oil; ¹H NMR (CDCl₃) δ 1.40 (s, 9H); 1.47 (d, J=6.9 Hz, 3H); 1.98 (brs, 2H); 2.49 (dd, J=7.9 Hz, J=17.7 Hz, 1H); 2.83 (dd, J=3.6 Hz, J=16.7 Hz, 1H); 3.69 (brs, 1H); 4.99-5.10 (m, 1H); 7.19-7.33 (m, 5H); 7.65-7.68 (m, 1H).

### Example 9J

D-aspartic acid β-t-butyl ester α-[(R)-α-methylbenzyl]amide. N-benzyloxycarbonyl-D-aspartic acid β-t-butyl ester α-[(R)-α-methylbenzyl]amide (0.273 g, 0.64 mmol) gave 0.187 g (quantitative yield) as an off-white oil; ¹H NMR (CDCl₃) δ 1.38 (s, 9H); 1.46 (d, J=6.9 Hz, 3H); 1.79 (brs, 2H); 2.51 (dd, J=7.8 Hz, J=17.5 Hz, 1H); 2.87 (dd, J=3.6 Hz, J=16.9 Hz, 1H); 4.19 (brs, 1H); 4.99-5.11 (m, 1H); 7.18-7.34 (m, 5H); 7.86-7.90 (m, 1H).

### Example 9K

D-aspartic acid β-t-butyl ester α-[N-methyl-N-(3-trifluoromethylbenzyl)]amide. N-benzyloxycarbonyl-D-aspartic acid β-t-butyl ester α-[N-methyl-N-(3-trifluoromethylbenzyl)]amide (0.282 g, 0.57 mmol) gave 0.195 g (95%) as an off-white oil.

### Example 9L

L-aspartic acid β-t-butyl ester α-[4-(2-phenylethyl)]piperazinamide. N-benzyloxycarbonyl-L-aspartic acid β-t-butyl ester α-[4-(2-phenylethyl)]piperazinamide (5.89 g, 11.9 mmol) gave 4.24 g (98%) as an off-white oil; ¹H NMR (CDCl₃): δ 1.42 (s, 9H); 2.61-2.95 (m, 10H); 3.60-3.90 (m, 4H); 4.35-4.45 (m, 1H); 7.17-7.29 (m, 5H).

### Example 9M

D-aspartic acid β-t-butyl ester α-(3-trifluoromethyl)benzylamide. N-benzyloxycarbonyl-D-aspartic acid β-t-butyl ester α-(3-trifluoromethyl)benzylamide (1.41 g, 2.93 mmol) gave 0.973 g (96%) as an off-white oil; ¹H NMR (CDCl₃): δ 1.42 (s, 9H); 2.21 (brs, 2H); 2.67 (dd, J=7.1 Hz, J=16.8 Hz, 1H); 2.84 (dd, J=3.6 Hz, J=16.7 Hz, 1H); 3.73-3.77 (m, 1H); 4.47-4.50 (m, 2H); 7.41-7.52 (m, 4H); 7.83-7.87 (m, 1H).

### Example 9N

L-glutamic acid γ-t-butyl ester α-(3-trifluoromethyl)benzylamide. N-benzyloxycarbonyl-L-glutamic acid γ-t-butyl ester α-(3-trifluoromethyl)benzylamide (5.41 g, 10.9 mmol) gave 3.94 g (quantitative yield) as an off-white oil; ¹H NMR (CDCl₃): δ 1.41 (s, 9H); 1.73-1.89 (m, 3H); 2.05-2.16 (m, 1H); 2.32-2.38 (m, 2H); 3.47 (dd, J=5.0 Hz, J=7.5 Hz, 1H); 4.47-4.49 (m, 2H); 7.36-7.54 (m, 4H); 7.69-7.77 (m, 1H).

### Example 9O

L-glutamic acid γ-t-butyl ester α-[4-(2-phenylethyl)]piperazinamide. N-benzyloxycarbonyl-L-glutamic acid γ-t-butyl ester α-[4-(2-phenylethyl)]piperazinamide (5.86 g, 11.50 mmol) gave 4.28 g (99%) as an off-white oil; ¹H NMR (CDCl₃) δ 1.39 (s, 9H); 2.00-2.08 (m, 1H); 2.38-2.46 (m, 1H); 2.55-2.90 (m, 9H); 3.61-3.82 (m, 4H); 4.48-4.56 (m, 1H); 7.17-7.26 (m, 5H).

### Example 9P

D-glutamic acid γ-t-butyl ester α-(3-trifluoromethyl)benzylamide. N-benzyloxycarbonyl-D-glutamic acid γ-t-butyl ester α-(3-trifluoromethyl)benzylamide (1.667 g, 3.37 mmol) gave 1.15 g (94%) as an off-white oil; ¹H NMR (CDCl₃) δ 1.41 (s, 9H); 1.80-2.20 (m, 4H); 2.31-2.40 (m, 2H); 3.51-3.59 (m, 1H); 4.47-4.49 (m, 2H); 7.39-7.52 (m, 4H); 7.71-7.79 (m, 1H).

### Example 9Q

L-glutamic acid α-t-butyl ester γ-(4-cyclohexyl)piperazinamide. N-Benzyloxycarbonyl-L-glutamic acid α-t-butyl ester γ-(4-cyclohexyl)piperazinamide (1.93 g, 3.96 mmol) gave 1.30 g (93%) as an off-white oil; ¹H NMR (CDCl₃) δ 1.02-1.25 (m, 5H); 1.41 (s, 9H); 1.45-1.50 (m, 1H); 1.56-1.60 (m, 1H); 1.69-1.80 (m, 6H); 3.30 (dd, J=4.8 Hz, J=8.5 Hz, 1H); 3.44 (t, J=9.9 Hz, 2H); 3.56 (t, J=9.9 Hz, 2H).

### Example 9R

D-aspartic acid β-t-butyl ester α-(2-fluoro-3-trifluoromethyl)benzylamide. N-benzyloxycarbonyl-D-aspartic acid β-t-butyl ester α-(2-fluoro-3-trifluoromethyl)benzylamide (0.36 g, 0.72 mmol) gave 0.256 g (92%) as an off-white oil; ¹H NMR (CDCl₃) δ 1.39 (s, 9H); 2.50 (brs, 2H); 2.74 (dd, J=7.0 Hz, J=16.5 Hz, 1H); 2.86 (dd, J=4.8 Hz, J=16.8 Hz, 1H); 3.89 (brs, 2H); 4.47-4.57 (m, 2H); 7.16 (t, J=7.8 Hz, 1H); 7.48 (t, J=7.3 Hz, 1H); 7.56 (t, J=7.3 Hz, 1H); 7.97-8.02 (m, 1H).

### Example 9S

D-aspartic acid β-t-butyl ester α-[(S)-1-(3-trifluoromethylphenyl)ethyl]amide. N-benzyloxycarbonyl-D-aspartic acid β-t-butyl ester α-[(S)-1-(3-trifluoromethylphenyl)ethyl]amide (120 mg, 0.24 mmol) gave 91 mg (91%) as an off-white oil.

### Example 9T

D-aspartic acid β-t-butyl ester α-[(R)-1-(3-trifluoromethylphenyl)ethyl]amide. N-benzyloxycarbonyl-D-aspartic acid β-t-butyl ester α-[(R)-1-(3-trifluoromethylphenyl)ethyl]amide (217 mg, 0.44 mmol) gave 158 mg (quantitative yield) as an off-white oil.

### Example 9U

D-aspartic acid β-t-butyl ester α-[N-methyl-N-(3-trifluoromethylbenzyl)]amide. N-benzyloxycarbonyl-D-aspartic acid β-t-butyl ester α-[N-methyl-N-(3-trifluoromethylbenzyl)]amide (0.282 g, 0.57 mmol) gave 0.195 g (95%) as an off-white oil.

### Example 9V

D-glutamic acid α-methyl ester γ-(3-trifluoromethyl)benzylamide. N-Benzyloxycarbonyl-D-glutamic acid α-methyl ester γ-(3-trifluoromethyl)benzylamide (764 mg, 1.69 mmol) gave g (516 mg, 96%) as an off-white oil.

### Example 14

### General Procedure for Formation of a 2-azetidinone from an Imine and an Acetyl Chloride

Step 1: General procedure for formation of an imine from an amino acid derivative. A solution of 1 equivalent of an α-amino acid ester or amide in dichloromethane is treated sequentially with 1 equivalent of an appropriate aldehyde, and a dessicating agent, such as magnesium sulfate or silica gel, in the amount of about 2 grams of dessicating agent per gram of starting α-amino acid ester or amide. The reaction is stirred at ambient temperature until all of the reactants are consumed as measured by thin layer chromatography. The reactions are typically complete within an hour. The reaction mixture is then filtered, the filter cake is washed with dichloromethane, and the filtrate concentrated under reduced pressure to give the desired imine that is used as is in the subsequent step.

Step 2: General procedure for the 2+2 cycloaddition of an imine and an acetyl chloride. A dichloromethane solution of the imine (10 mL dichloromethane/1 gram imine) is cooled to 0° C. To this cooled solution is added 1.5 equivalents of an appropriate amine, typically triethylamine, followed by the dropwise addition of a dichloromethane solution of 1.1 equivalents of an appropriate acetyl chloride, such as that described in Example 1A (10 mL dichloromethane/1 gm appropriate acetyl chloride). The reaction mixture is allowed to warm to ambient temperature over 1 h and is then quenched by the addition of a saturated aqueous solution of ammonium chloride. The resulting mixture is partitioned between water and dichloromethane. The layers are separated and the organic layer is washed successively with 1N hydrochloric acid, saturated aqueous sodium bicarbonate, and saturated aqueous sodium chloride. The organic layer is dried over magnesium sulfate and concentrated under reduced pressure. The residue may be used directly for further reactions, or purified by chromatography or by crystallization from an appropriate solvent system if desired.

### Example 15

General procedure for α-alkylation and/or acylation of an (azetidin-2-on-1-yl)acetate. A solution of (azetidin-2-on-1-yl)acetate in tetrahydrofuran (ca. 0.22 M in azetidinone), such as Example 16A, is cooled to −78° C. and treated with lithium bis(trimethylsilyl)amide (2.2 equivalents). The resulting anion is treated with an appropriate alkyl or acyl halide (1.1 equivalents). Upon complete conversion of the azetidinone, the reaction is quenched with saturated aqueous ammonium chloride and partitioned between ethyl acetate and water. The organic phase is washed sequentially with 1N HCl, saturated aqueous sodium bicarbonate, and saturated aqueous NaCl. The resulting organic layer is dried (e.g. magnesium sulfate) and evaporated. The residue is purified by silica gel chromatography with an appropriate eluent, such as 3:2 hexane/ethyl acetate.

### Example 16A

tert-Butyl[3(S)-(4(S)-phenyloxazolidin-2-on-3-yl)-4(R)-(2-styryl)azetidin-2-on-1-yl]acetate. Prepared according to Example 14, the imine prepared from 4.53 g (34.5 mmol) glycine tert-butyl ester and cinnamaldehyde was combined with 2-(4(S)-phenyloxazolidin-2-on-3-yl)acetyl chloride (Example 1A) to give 5.5 g (30%) as colorless crystals (recrystallized, n-chlorobutane); mp 194-195° C.

### Example 17

2,2,2-Trichloroethyl 2(RS)-(tert-butoxycarbonyl)-2-[3(S)-(4(S)-phenyloxazolidin-2-on-3-yl)-4(R)-(2-styryl)azetidin-2-on-1-yl]acetate. Prepared according to Example 15, 9.0 g (20 mmol) of Example 16A was acylated with 4.2 g (20 mmol) of trichloroethylchloroformate to give 7.0 g (56%); mp 176-178° C.

### Example 18A

2(RS)-(tert-Butoxycarbonyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. A solution of 0.20 g (0.32 mmol) of Example 17 and 52 μL (0.36 mmol) of (3-trifluoromethylbenzyl)amine in THF was heated at reflux. Upon complete conversion (TLC), the solvent was evaporated and the residue was recrystallized (chloroform/hexane) to give 0.17 g (82%) as a white solid; mp 182-184° C.

### Example 18B

2(RS)-(tert-Butoxycarbonyl)-2-[3(S)-(4(S)-phenyloxazolidin-2-on-3-yl)-4(R)-(2-styryl)azetidin-2-on-1-yl]acetic acid N-(2-fluoro-3-trifluoromethylbenzyl)amide. Prepared according to Example 18A, using 2-fluoro-3-(trifluoromethyl)benzylamine instead of (3-trifluoromethylbenzyl)amine to give a white solid (140 mg, 41%).

### Examples 19

Were prepared according to the procedure of Example 14, where the appropriate amino acid derivative and aldehyde were used in Step 1, and the appropriate acetyl chloride was used in Step 2.

### Example 19A

2(S)-(tert-Butoxycarbonylmethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. The imine prepared from 1.52 g (4.39 mmol) of L-aspartic acid β-t-butyl ester α-(3-trifluoromethyl)benzylamide and cinnamaldehyde was combined with 2-(4(S)-phenyloxazolidin-2-on-3-yl)acetyl chloride (Example 1A) to give 2.94 g of an orange-brown oil that gave, after flash column chromatography purification (70:30 hexanes/ethyl acetate), 2.06 g (70%) of Example 19 as a white solid; ¹H NMR (CDCl₃) δ 1.39 (s, 9H); 2.46 (dd, J=11.1 Hz, J=16.3 Hz, 1H); 3.18 (dd, J=3.8 Hz, J=16.4 Hz, 1H); 4.12-4.17 (m, 1H); 4.26 (d, J=5.0 Hz, 1H); 4.45 (dd, J=6.0 Hz, J=14.9 Hz, 1H); 4.54 (dd, J=5.3 Hz, J=9.8 Hz, 1H); 4.58-4.66 (m, 3H); 4.69-4.75 (m, 1H); 4.81 (dd, J=3.8 Hz, J=11.1 Hz, 1H); 6.25 (dd, J=9.6 Hz, J=15.8 Hz, 1H); 6.70 (d, J=15.8 Hz, 1H); 7.14-7.17 (m, 2H); 7.28-7.46 (m, 11H); 7.62 (s, 1H); 8.27-8.32 (m, 1H).

### Example 19B

2(S)-(tert-Butoxycarbonylmethyl)-2-[3(R)-(4(R)-phenyloxazolidin-2-on-3-yl)-4(S)-(2-styryl)azetidin-2-on-1-yl]acetic acid N-(3-trifluoromethylbenzyl)amide. Example 19A was prepared according to the method of Example 19A except that 2-(4(R)-phenyloxazolidin-2-on-3-yl)acetyl chloride

(Example 1B) was used instead of 2-(4(S)-phenyloxazolidin-2-on-3-yl)acetyl chloride to give a white solid (41 mg, 13%); ¹H NMR (CDCl₃) δ 1.37 (s, 9H); 3.11 (dd, J=3.7 Hz, J=17.8 Hz, 1H); 3.20 (dd, J=10.6 Hz, J=17.8 Hz, 1H); 4.02 (dd, J=3.7 Hz, J=10.6 Hz, 1H); 4.10-4.17 (m, 1H); 4.24 (d, J=4.9 Hz, 1H); 4.4652-4.574 (dd, J=5.9 Hz, J=15.1 Hz, 1H); 4.58-4.76 (m, 4H); 6.27 (dd, J=9.6 Hz, J=15.8 Hz, 1H); 6.79 (d, J=15.8 Hz, 1H); 7.23-7.53 (m, 13H); 7.63 (s, 1H); 8.51-8.55 (m, 1H).

### Example 19C

2(S)-(tert-Butoxycarbonylethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. The imine prepared from 3.94 g (10.93 mmol) of L-glutamic acid γ-t-butyl ester α-(3-trifluoromethyl)benzylamide and cinnamaldehyde was combined with 2-(4(S)-phenyloxazolidin-2-on-3-yl)acetyl chloride (Example 1A) to give 5.53 g (75%) after flash column chromatography purification (70:30 hexanes/ethyl acetate); ¹H NMR (CDCl₃) δ 1.36 (s, 9H); 1.85-1.96 (m, 1H); 2.18-2.49 (m, 3H); 4.14-4.19 (m, 1H); 4.30 (d, J=4.9 Hz, 2H); 4.44 (dd, J=6.1 Hz, J=14.9 Hz, 1H); 4.56-4.67 (m, 4H); 4.71-4.75 (m, 1H); 6.26 (dd, J=9.6 Hz, J=15.8 Hz, 1H); 6.71 (d, J=15.8 Hz, 1H); 7.16-7.18 (m, 2H); 7.27-7.49 (m, lI1H); 7.60 (s, 1H); 8.08-8.12 (m, 1H).

### Example 19D

2(S)-(tert-Butoxycarbonylmethyl)-2-[3 (S)-(4(S)-phenyloxazolidin-2-on-3-yl)-4(R)-(2-styryl)azetidin-2-on-1-yl]acetic acid N-[4-(2-phenylethyl)]piperazinamide. The imine prepared from 4.20 g (11.6 mmol) of L-aspartic acid β-t-butyl ester α-[4-(2-phenylethyl)]piperazinamide and cinnamaldehyde was combined with 2-(4(S)-phenyloxazolidin-2-on-3-yl)acetyl chloride (Example 1A) to give 4.37 g (55%) after flash column chromatography purification (50:50 hexanes/ethyl acetate); ¹H NMR (CDCl₃) δ 1.34 (s, 9H); 2.26-2.32 (m, 1H); 2.46-2.63 (m, 4H); 2.75-2.89 (m, 4H); 3.24-3.32 (m, 1H); 3.49-3.76 (m, 3H); 4.07-4.13 (m, 1H); 4.30 (d, J=4.6 Hz, 1H); 4.22-4.48 (m, 1H); 4.55-4.61 (m, 1H); 4.69-4.75 (m, 1H); 5.04-5.09 (m, 1H); 6.15 (dd, J=9.3 Hz, J=15.9 Hz, 1H); 6.63 (d, J=15.8 Hz, 1H); 7.18-7.42 (m, 15H).

### Example 19E

2(S)-(tert-Butoxycarbonylethyl)-2-[3(S)-(4(S)-phenyloxazolidin-2-on-3-yl)-4(R)-(2-styryl)azetidin-2-on-1-yl]acetic acid N-[4-(2-phenylethyl)]piperazinamide. The imine prepared from 2.54 g (6.75 mmol) of L-glutamic acid γ-t-butyl ester α-[4-(2-phenylethyl)]piperazinamide and cinnamaldehyde was combined with 2-(4(S)-phenyloxazolidin-2-on-3-yl)acetyl chloride (Example 1A) to give 3.55 g (76%) after flash column chromatography purification (50:50 hexanes/ethyl acetate); ¹H NMR (CDCl₃) δ 1.32 (s, 9H); 1.96-2.07 (m, 1H); 2.15-2.44 (m, 6H); 2.54-2.62 (m, 2H); 2.69-2.81 (m, 3H); 3.28-3.34 (m, 1H); 3.59-3.68 (m, 1H); 4.08-4.13 (m, 1H); 4.33-4.44 (m, 2H); 4.48-4.60 (m, 2H); 4.67-4.77 (m, 1H); 6.14 (dd, J=8.9 Hz, J=16.0 Hz, 1H); 6.62 (d, J=16.0 Hz, 1H); 7.16-7.42 (m, 15H).

### Example 19F

2(R)-(tert-Butoxycarbonylmethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. The imine prepared from 0.973 g (2.81 mmol) of D-aspartic acid β-t-butyl ester α-(3-trifluoromethyl)benzylamide and cinnamaldehyde was combined with 2-(4(S)-phenyloxazolidin-2-on-3-yl)acetyl chloride (Example 1A) to give 1.53 g (82%) after flash column chromatography purification (70:30 hexanes/ethyl acetate); ¹H NMR (CDCl₃) δ 1.37 (s, 9H); 3.10 (dd, J=3.7 Hz, J=17.8 Hz, 1H); 3.20 (dd, J=10.7 Hz, J=17.8 Hz, 1H); 4.02 (dd, J=3.6 Hz, J=10.6 Hz, 1H); 4.11-4.17 (m, 1H); 4.24 (d, J=4.9 Hz, 1H); 4.46 (dd, J=5.8 Hz, J=15.1 Hz, 1H); 4.58-4.67 (m, 3H); 4.70-4.76 (m, 1H); 6.27 (dd, J=9.5 Hz, J=15.8 Hz, 1H); 6.79 (d, J=15.8 Hz, 1H); 7.25-7.50 (m, 13H); 7.63 (s, 1H); 8.50-8.54 (m, if).

### Example 19G

2(R)-(tert-Butoxycarbonylmethyl)-2-[3(R)-(4(R)-phenyloxazolidin-2-on-3-yl)-4(S)-(2-styryl)azetidin-2-on-1-yl]acetic acid N-(3-trifluoromethylbenzyl)amide. Prepared according to Example 19F except that 2-(4(R)-phenyloxazolidin-2-on-3-yl)acetyl chloride (Example 1B) was used instead of 2-(4(S)-phenyloxazolidin-2-on-3-yl)acetyl chloride to give a white solid (588 mg, 49%); ¹H NMR (CDCl₃) δ 1.39 (s, 9H); 2.47 (dd, J=11.2 Hz, J=16.3 Hz, 1H); 3.18 (dd, J=3.8 Hz, J=16.3 Hz, 1H); 4.15 (t, J=8.25, Hz 1H); 4.26 (d, J=5.0 Hz, 1H); 4.45 (dd, J=6.0 Hz, J=15.0 Hz, 1H); 4.52-4.57 (m, 3H); 4.63 (t, J=9 Hz, 1H); 4.70 (t, J=8 Hz, 1H); 4.81 (dd, J=3.8 Hz, J=10.8 Hz, 1H); 6.25 (dd, J=9.8 Hz, J=15.8 Hz, 111); 6.70 (d, J=15.8 Hz, 1H); 7.15-7.17 (m, 2H); 7.27-7.51 (m, 1H); 7.62 (s, 1H); 8.27-8.32 (m, 1H).

### Example 19H

2(R)-(tert-Butoxycarbonylethyl)-2-[3 (S)-(4(S)-phenyloxazolidin-2-on-3-yl)-4(R)-(2-styryl)azetidin-2-on-1-yl]acetic acid N-(3-trifluoromethylbenzyl)amide. The imine prepared from 1.15 g (3.20 mmol) of D-glutamic acid γ-t-butyl ester α-(3-trifluoromethyl)benzylamide and cinnamaldehyde was combined with 2-(4(S)-phenyloxazolidin-2-on-3-yl)acetyl chloride (Example 1A) to give 1.84 g (85%) after flash column chromatography purification (70:30 hexanes/ethyl acetate); ¹H NMR (CDCl₃) δ 1.37 (s, 9H); 2.23-2.39 (m, 4H); 3.71-3.75 (m, 1H); 4.13-4.18 (m, 1H); 4.31 (d, J=4.9 Hz, 1H); 4.44-4.51 (m, 2H); 4.56-4.68 (m, 2H); 4.71-4.76 (m, 1H); 6.26 (dd, J=9.5 Hz, J=15.8 Hz, 1H); 6.71 (d, J=15.8 Hz, 1H); 7.25-7.52 (m, 13H); 7.63 (s, 1H); 8.25-8.30 (m, 1H).

### Example 19I

2(S)-(tert-Butoxycarbonylethyl)-2-[3(S)-(4(S)-phenyloxazolidin-2-on-3-yl)-4(R)-(2-styryl)azetidin-2-on-1-yl]acetic acid N-(4-cyclohexyl)piperazinamide. The imine prepared from 2.58 g (5.94 mmol) of L-glutamic acid γ-t-butyl ester α-(4-cyclohexyl)piperazinamide and cinnamaldehyde was combined with 2-(4(S)-phenyloxazolidin-2-on-3-yl)acetyl chloride (Example 1A) to give 3.27 g (94%) after flash column chromatography purification (95:5 dichloromethane/methanol); ¹H NMR (CDCl₃) δ 1.32 (s, 9H); 1.10-1.18 (m, 1H); 1.20-1.31 (m, 2H); 1.38-1.45 (m, 2H); 1.61-1.66 (m, 1H); 1.84-1.89 (m, 2H); 1.95-2.01 (m, 1H); 2.04-2.14 (m, 3H); 2.20-2.24 (m, 1H); 2.29-2.35 (m, 1H); 2.85-2.92 (m, 1H); 3.24-3.32 (m, 1H); 3.36-3.45 (m, 2H); 3.80-3.86 (m, 1H); 4.08 (t, J=8.3 Hz, 1H); 4.27 (d, J=5.0 Hz, 1H); 4.31-4.55 (m, 4H); 4.71 (t, J=8.3 Hz, 1H); 4.83-4.90 (m, 1H); 6.18 (dd, J=9.1 Hz, J=15.9 Hz, 1H); 6.67 (d, J=15.9 Hz, 1H); 7.25-7.44 (m, 10H); 8.22 (brs, 1H).

### Example 19J

Tert-Butyl 2(S)-(2-(4-cyclohexylpiperazinylcarbonyl)ethyl)-2-[3(S)-(4(S)-phenyloxazolidin-2-on-3-yl)-4(R)-(2-styryl)azetidin-2-on-1-yl]acetate. The imine prepared from 1.282 g (3.63 mmol) of L-glutamic acid α-t-butyl ester γ-(4-cyclohexyl)piperazinamide and cinnamaldehyde was combined with 2-(4(S)-phenyloxazolidin-2-on-3-yl)acetyl chloride (Example 1A) to give 1.946 g (80%) after flash column chromatography purification (50:50 hexanes/ethyl acetate); ¹H NMR (CDCl₃) δ 1.15-1.26 (m, 6H); 1.39 (s, 9H); 1.55-1.64 (m, 2H); 1.77-1.83 (m, 3H); 2.22-2.35 (m, 2H); 2.40-2.50 (m, 6H); 2.75-2.79 (m, 1H); 3.43-3.48 (m, 1H); 3.56-3.60 (m, 2H); 3.75-3.79 (m, 1H); 4.10 (t, J=8.3 Hz, 1H); 4.31-4.35 (m, 2H); 4.58 (t, J=8.8 Hz, 1H); 4.73 (t, J=8.4 Hz, 1H); 6.17 (dd, J=8.6 Hz, J=16.0 Hz, 1H); 6.65 (d, J=16.0 Hz, 1H); 7.27-7.42 (m, 10H).

### Example 19K

2(R)-(tert-Butoxycarbonylmethyl)-2-[3(S)-(4(S)-phenyloxazolidin-2-on-3-yl)-4(R)-(2-styryl)azetidin-2-on-1-yl]acetic acid N-(2-fluoro-3-trifluoromethylbenzyl)amide. The imine prepared from 0.256 g (0.70 mmol) of D-aspartic acid β-t-butyl ester α-(2-fluoro-3-trifluoromethyl)benzylamide and cinnamaldehyde was combined with 2-(4(S)-phenyloxazolidin-2-on-3-yl)acetyl chloride (Example 1A) to give 0.287 g (60%) after flash column chromatography purification (70:30 hexanes/ethyl acetate); ¹H NMR (CDCl₃) δ 1.38 (s, 9H); 3.12 (dd, J=4.0 Hz, J=17.8 Hz, 1H); 3.20 (dd, J=10.4 Hz, J=17.8 Hz, 1H); 4.05 (dd, J=3.9 Hz, J=10.4 Hz, 1H); 4.14 (dd, J=J′=8.2 Hz, 1H); 4.25 (d, J=4.9 Hz, 1H); 4.59-4.67 (m, 4H); 4.74 (t, J=8.3 Hz, 1H); 6.36 (dd, J=9.6 Hz, J=15.8 Hz, 1H); 6.83 (d, J=15.8 Hz, 1H); 7.02-7.07 (m, 1H); 7.28-7.55 (m, 12H); 8.44-8.48 (m, 1H).

### Example 19L

2(R)-(tert-Butoxycarbonylmethyl)-2-[3(S)-(4(S)-phenyloxazolidin-2-on-3-yl)-4(R)-(2-styryl)azetidin-2-on-1-yl]acetic acid N—[(S)-α-methylbenzyl]amide. The imine prepared from 0.167 g (0.57 mmol) of D-aspartic acid β-t-butyl ester [(S)-α-methylbenzyl]amide and cinnamaldehyde was combined with 2-(4(S)-phenyloxazolidin-2-on-3-yl)acetyl chloride (Example 1A) to give 0.219 g (63%) after flash column chromatography purification (70:30 hexanes/ethyl acetate); ¹H NMR (CDCl₃) δ 1.35 (s, 9H); 1.56 (d, J=7.0 Hz, 3H); 2.97 (dd, J=3.5 Hz, J=18.0 Hz, 1H); 3.15 (dd, J=11.0 Hz, J=17.5 Hz, 1H); 4.01 (dd, J=3.0 Hz, J=11.0 Hz, 1H); 4.14 (t, J=8.5 Hz, 1H); 4.24 (d, J=5.0 Hz, 1H); 4.57 (dd, J=5.0 Hz, J=9.5 Hz, 1H); 4.64 (t, J=8.8 Hz, 1H); 5.07 (t, J=8.5 Hz, 1H); 5.03-5.09 (m, 1H); 6.43 (dd, J=9.5 Hz, J=16.0 Hz, 1H); 6.83 (d, J=16.0 Hz, 1H); 7.16-7.20 (m, 1H); 7.27-7.49 (m, 14H); 8.07-8.10 (m, 1H).

### Example 19M

2(R)-(tert-Butoxycarbonylmethyl)-2-[3(S)-(4(S)-phenyloxazolidin-2-on-3-yl)-4(R)-(2-styryl)azetidin-2-on-1-yl]acetic acid N—[(R)-α-methylbenzyl]amide. The imine prepared from 0.187 g (0.46 mmol) of D-aspartic acid β-t-butyl ester [(R)-α-methylbenzyl]amide and cinnamaldehyde was combined with 2-(4(S)-phenyloxazolidin-2-on-3-yl)acetyl chloride (Example 1A) to give 0.25 g (64%) after flash column chromatography purification (70:30 hexanes/ethyl acetate); ¹H NMR (CDCl₃) δ 1.36 (s, 9H); 1.59 (d, J=7.1 Hz, 3H); 3.10 (dd, J=3.5 Hz, J=17.8 Hz, 1H); 3.22 (dd, J=10.9 Hz, J=17.8 Hz, 1H); 3.93 (dd, J=3.5 Hz, J=10.8 Hz, 1H); 4.14 (t, J=8.1 Hz, 1H); 4.24 (d, J=5.0 Hz, 1H); 4.58 (dd, J=5.0 Hz, J=9.5 Hz, 1H); 4.65 (t, J=8.7 Hz, 1H); 4.74 (t, J=8.2 Hz, 1H); 5.06-5.14 (m, 1H); 6.32 (dd, J=9.5 Hz, J=15.8 Hz, 1H); 6.74 (d, J=15.8 Hz, 1H); 7.19-7.43 (m, 15H); 8.15-8.18 (m, 1H).

### Example 19N

2(R)-(tert-Butoxycarbonylmethyl)-2-[3 (S)-(4(S)-phenyloxazolidin-2-on-3-yl)-4(R)-(2-styryl)azetidin-2-on-1-yl]acetic acid N-methyl-N-(3-trifluoromethylbenzyl)amide. The imine prepared from 0.195 g (0.41 mmol) of D-aspartic acid β-t-butyl ester α-[N-methyl-N-(3-trifluoromethylbenzyl)]amide and cinnamaldehyde was combined with 2-(4(S)-phenyloxazolidin-2-on-3-yl)acetyl chloride (Example 1A) to give 0.253 g (69%) after flash column chromatography purification (70:30 hexanes/ethyl acetate); ¹H NMR (CDCl₃) δ 1.36 (s, 9H); 2.53 (dd, J=4.0 Hz, J=17.0 Hz, 1H); 3.06 (dd, J=10.8 Hz, J=16.8 Hz, 1H); 3.13 (s, 3H); 4.12 (dd, J=8.0 Hz, J=9.0 Hz, 1H); 4.26 (d, J=5.0 Hz, 1H); 4.38 (d, J=15.0 Hz, 1H); 4.46 (dd, J=5.0 Hz, J=9.5 Hz, 1H); 4.56 (t, J=6.8 Hz, 1H); 4.70-4.79 (m, 2H); 5.27 (dd, J=4.0 Hz, J=11.0 Hz, 1H); 6.22 (dd, J=9.3 Hz, J=15.8 Hz, 1H); 6.73 (d, J=15.8 Hz, 1H); 7.33-7.45 (m, 14H).

### Example 19O

2(S)-(tert-Butoxycarbonylethyl)-2-[3 (S)-(4(S)-phenyloxazolidin-2-on-3-yl)-4(R)-(2-chloro-1-phenylethen-2-yl)azetidin-2-on-1-yl]acetic acid N-(3-trifluoromethylbenzyl)amide. The imine prepared from 1.62 g (4.44 mmol) of L-glutamic acid γ-t-butyl ester α-(3-trifluoromethyl)benzylamide and α-chlorocinnamaldehyde was combined with 2-(4(S)-phenyloxazolidin-2-on-3-yl)acetyl chloride (Example 1A) to give 0.708 g (22%) after flash column chromatography purification (70:30 hexanes/ethyl acetate); ¹H NMR (CDCl₃) δ 1.35 (s, 9H); 1.68 (brs, 1H); 2.19-2.35 (m, 2H); 2.40-2.61 (m, 2H); 4.13 (dd, J=7.5 Hz, J=9.0 Hz, 1H); 4.22 (t, J=7.0 Hz, 1H); 4.34 (d, J=4.5 Hz, 1H); 4.45 (dd, J=5.5 Hz, J=15.0 Hz, 1H); 4.51-4.60 (m, 3H); 4.89 (dd, J=7.5 Hz, J=8.5 Hz, 1H); 6.89 (s, 1H); 7.28-7.54 (m, 14H).

### Example 19P

2(R)-(tert-Butoxycarbonylmethyl)-2-[3(S)-(4(S)-phenyloxazolidin-2-on-3-yl)-4(R)-(1-(2-methoxyphenyl)ethen-2-yl)azetidin-2-on-1-yl]acetic acid N-(3-trifluoromethylbenzyl)amide. The imine prepared from 0.34 g (0.98 mmol) of D-aspartic acid β-t-butyl ester α-(3-trifluoromethylbenzyl)amide and 2′-methoxycinnamaldehyde was combined with 2-(4(S)-phenyloxazolidin-2-on-3-yl)acetyl chloride (Example 1A) to give 0.402 g (59%) after flash column chromatography purification (70:30 hexanes/ethyl acetate); ¹H NMR (CDCl₃) δ 1.35 (s, 9H); 1.68 (brs, 1H); 2.19-2.35 (m, 2H); 2.40-2.61 (m, 2H); 4.13 (dd, J=7.5 Hz, J=9.0 Hz, 1H); 4.22 (t, J=7.0 Hz, 1H); 4.34 (d, J=4.5 Hz, 1H); 4.45 (dd, J=5.5 Hz, J=15.0 Hz, 1H); 4.51-4.60 (m, 3H); 4.89 (dd, J=7.5 Hz, J=8.5 Hz, 1H); 6.89 (s, 1H); 7.28-7.54 (m, 14H).

### Example 19Q

tert-Butyl (2R)-(Benzyloxymethyl)-2-[3(S)-(4(S)-phenyloxazolidin-2-on-3-yl)-4(R)-(2-styryl)azetidin-2-on-1-yl]acetate. The imine prepared from 0.329 g (1.31 mmol) of O-(benzyl)-D-serine t-butyl ester (Example 8A) and cinnamaldehyde was combined with 2-(4(S)-phenyloxazolidin-2-on-3-yl)acetyl chloride (Example 1A) to give 0.543 g (73%) after flash column chromatography purification (90:10 hexanes/ethyl acetate); ¹H NMR (CDCl₃) δ 1.39 (s, 9H); 3.56 (dd, J=2.7 Hz, J=9.5 Hz, 1H); 3.82 (dd, J=4.8 Hz, J=9.5 Hz, 1H); 4.11 (t, J=8.3 Hz, 1H); 4.21-4.29 (m, 2H); 4.50-4.58 (m, 3H); 4.71-4.78 (m, 2H); 6.19 (dd, J=9.1 Hz, J=16.0 Hz, 1H); 6.49 (d, J=16.0 Hz, 1H); 7.07-7.11 (m, 1H); 7.19-7.40 (m, 14H).

### Example 19R

tert-Butyl 2(S)-(2-(4-cyclohexylpiperazinylcarbonyl)methyl)-2-[3(S)-(4(S)-phenyloxazolidin-2-on-3-yl)-4(R)-(2-styryl)azetidin-2-on-1-yl]acetate. The imine prepared from 0.3 g (0.88 mmol) of L-aspartic acid α-t-butyl ester γ-(4-cyclohexyl)piperazinamide and cinnamaldehyde was combined with 2-(4(S)-phenyloxazolidin-2-on-3-yl)acetyl chloride (Example 1A) to give 464 mg (80%) as a white solid after flash column chromatography purification (50:50 hexanes/ethyl acetate).

### Example 19S

tert-Butyl 3(R)-[3(S)-(4(S)-phenyloxazolidin-2-on-3-yl)-3-methyl-4(R)-(styr-2-yl)azetidin-2-on-1-yl]-3-[(3-trifluoromethyl)phenylmethylaminocarbonyl]propanoate. The imine prepared from 0.307 g (0.89 mmol) of D-aspartic acid β-t-butyl ester α-(3-trifluoromethyl)benzylamide (Example 19C) and cinnamaldehyde was combined with 2-(4(S)-phenyloxazolidin-2-on-3-yl)propanoyl chloride (Example 1C) to give 120 mg (20%) after flash column chromatography purification (hexanes 70%/EtOAc 30%); ¹H NMR (CDCl₃) δ 1.25 (s, 3H), 1.38 (s, 9H); 3.09 (dd, J=3.0 Hz, J=18.0 Hz, 1H); 3.33 (dd, J=12.5 Hz, J=18.0 Hz, 1H); 4.01 (dd, J=3.0 Hz, J=11.5 Hz, 1H); 4.04 (dd, J=3.5 Hz, J=8.8 Hz, 1H); 4.42 (d, J=9.0 Hz, 1H); 4.45-4.51 (m, 3H); 4.61-4.66 (m, 1H); 4.75 (dd, J=3.5 Hz, J=8.5 Hz, 1H); 6.23 (dd, J=9.0 Hz, J=15.5 Hz, 1H); 6.78 (d, J=15.5 Hz, 1H); 7.23-7.53 (m, 13H); 7.64 (s, 1H).

### Example 19T

2(R)-(tert-Butoxycarbonylmethyl)-2-[3(S)-(4(S)-phenyloxazolidin-2-on-3-yl)-4(R)-(prop-1-enyl)azetidin-2-on-1-yl]acetic acid N-(3-trifluoromethylbenzyl)amide. The imine prepared from 0.289 g (0.83 mmol) of D-aspartic acid β-t-butyl ester α-(3-trifluoromethyl)benzylamide and crotonaldehyde was combined with 2-(4(S)-phenyloxazolidin-2-on-3-yl)acetyl chloride (Example 1A) to give 381 mg (76%) after flash column chromatography purification (99:1 CH₂Cl₂/MeOH); ¹H NMR (CDCl₃) δ 1.36 (s, 9H), 1.69 (dd, J=2 Hz, J=6.5 Hz, 3H); 3.08 (dd, J=3.3 Hz, J=17.8 Hz, 1H); 3.18 (dd, J=1 Hz, J=17.5 Hz, 1H); 3.94 (dd, J=3.5 Hz, J=11 Hz, 1H); 4.12 (d, J=5 Hz, 1H); 4.15 (dd, J=7 Hz, J=8 Hz, 1H); 4.35 (dd, J=4.8 Hz, J=9.8 Hz, 1H); 4.44 (dd, J=6 Hz, J=15 Hz, 1H); 4.61 (dd, J=6 Hz, J=15 Hz, 1H); 4.67-4.75 (m, 2H); 5.52-5.58 (m, 1H); 5.92-6.00 (m, 1H); 7.33-7.60 (m, 9H); 8.47-8.50 (m, 1H).

### Example 19U

Methyl 2(S)-(tert-Butoxycarbonylethyl)-2-[3(S)-(4(S)-phenyloxazolidin-2-on-3-yl)-4(R)-(2-styryl)azetidin-2-on-1-yl]acetate. The imine prepared from 433 mg (1.99 mmol) of L-glutamic acid γ-t-butyl ester α-methyl ester and cinnamaldehyde was combined with 2-(4(S)-phenyloxazolidin-2-on-3-yl)acetyl chloride (Example 1A) to give 682 mg (64%) after flash column chromatography purification (70:30 hexanes/ethyl acetate); ¹H NMR (CDCl₃) δ 1.32 (s, 9H); 2.10-2.26 (m, 1H); 2.30-2.41 (m, 3H); 3.66 (s, 3H); 3.95-3.99 (m, 1H); 4.16 (dd, J=7.5 Hz, J=9 Hz, 1H); 4.38 (dd, J=5 Hz, J=9 Hz, 1H); 4.55 (d, J=5 Hz 1H); 4.61 (t, J=9 Hz, 1H); 4.86 (dd, J=7.5 Hz, J=9 Hz, 1H); 6.00 (dd, J=9 Hz, J=16 Hz, 1H); 6.60 (d, J=16 Hz, 1H); 7.26-7.43 (m, 10H).

### Example 19V

Tert-Butyl 2(S)-(methoxycarbonylethyl)-2-[3(S)-(4(S)-phenyloxazolidin-2-on-3-yl)-4(R)-(2-styryl)azetidin-2-on-1-yl]acetate. The imine prepared from 428 mg (1.97 mmol) of L-glutamic acid γ-t-butyl ester α-methyl ester and cinnamaldehyde was combined with 2-(4(S)-phenyloxazolidin-2-on-3-yl)acetyl chloride (Example 1A) to give 864 mg (82%) after flash column chromatography purification (70:30 hexanes/ethyl acetate); ¹H NMR (CDCl₃) δ 1.40 (s, 9H); 2.12-2.27 (m, 1H); 2.32-2.55 (m, 3H); 3.50 (s, 3H); 3.72 (dd, J=4.6 Hz, J=10.4 Hz, 1H); 4.12-4.17 (m, 1H); 4.34 (dd, J=5 Hz, J=9 Hz, 1H); 4.50 (d, J=5 Hz, 1H); 4.60 (t, J=8.9 Hz, 1H); 4.81-4.86 (m, 1H); 6.06 (dd, J=9 Hz, J=16 Hz, 1H); 6.59 (d, J=16 Hz, 1H); 7.25-7.42 (m, 10H).

### Example 19W

Methyl 2(S)-(tert-Butoxycarbonylmethyl)-2-[3(S)-(4(S)-phenyloxazolidin-2-on-3-yl)-4(R)-(2-styryl)azetidin-2-on-1-yl]acetate. The imine prepared from 424 mg (2.09 mmol) of L-aspartic acid γ-t-butyl ester α-methyl ester and cinnamaldehyde was combined with 2-(4(S)-phenyloxazolidin-2-on-3-yl)acetyl chloride (Example 1A) to give 923 mg (85%) after recrystallization from CH₂Cl₂/hexanes; ¹H NMR (CDCl₃) δ 1.41 (s, 9H); 2.77 (dd, J=7.5 Hz, J=16.5 Hz, 1H); 3.00 (dd, J=7 Hz, J=16.5 Hz, 1H); 4.16 (dd, J=7.5 Hz, J=9 Hz, 1H); 4.41-48 (m, 2H); 4.55 (d, J=5 Hz, 1H); 4.60 (t, J=8.8 Hz, 1H); 4.86 (dd, J=7.5 Hz, J=9 Hz, 1H); 5.93 (dd, J=9.5 Hz, J=15.5 Hz, 1H); 6.61 (d, J=15.5 Hz, 1H); 7.25-7.43 (m, 10H).

### Example 19X

2(R)-(tert-Butoxycarbonylmethyl)-2-[3(S)-(4(S)-phenyloxazolidin-2-on-3-yl)-4(R)-(2-styryl)azetidin-2-on-1-yl]acetic acid N—[(R)-1-(3-trifluoromethylphenyl)ethyl]amide. The imine prepared from 160 mg (0.44 mmol) of D-aspartic acid β-t-butyl ester α-[(R)-1-(3-trifluoromethylphenyl)ethyl]amide and cinnamaldehyde was combined with 2-(4(S)-phenyloxazolidin-2-on-3-yl)acetyl chloride (Example 1A) to give 166 mg (55%) after flash column chromatography purification (70:30 hexanes/EtOAc).

### Example 19Y

2(R)-(tert-Butoxycarbonylmethyl)-2-[3 (S)-(4(S)-phenyloxazolidin-2-on-3-yl)-4(R)-(2-styryl)azetidin-2-on-1-yl]acetic acid N—[(S)-1-(3-trifluoromethylphenyl)ethyl]amide. The imine prepared from 120 mg (0.22 mmol) of D-aspartic acid β-t-butyl ester α-[(S)-1-(3-trifluoromethylphenyl)ethyl]amide and cinnamaldehyde was combined with 2-(4(S)-phenyloxazolidin-2-on-3-yl)acetyl chloride (Example 1A) to give 75 mg (50%) after flash column chromatography purification (70:30 hexanes/EtOAc).

### Example 19Z

Methyl 2(R)-(2-(3-trifluoromethylbenzyl)aminocarbonyl)ethyl)-2-[3(S)-(4(S)-phenyloxazolidin-2-on-3-yl)-4(R)-(2-styryl)azetidin-2-on-1-yl]acetate. The imine prepared from 517 mg (1.62 mmol) of D-glutamic acid α-methyl ester γ-(3-trifluoromethyl)benzylamide and cinnamaldehyde was combined with 2-(4(S)-phenyloxazolidin-2-on-3-yl)acetyl chloride (Example 1A) to give 527 mg (51%) after flash column chromatography purification (50:50 hexanes/EtOAc).

The following Examples 20 were prepared according to the processes described herein:

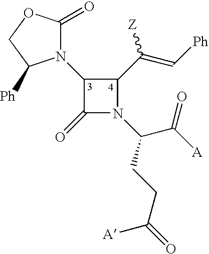

| Example | Stereochemistry | Y | A | A′ |
|---|---|---|---|---|
| 20A | (3S,4R) | F | 3-CF₃-benzylamino | t-butoxy |
| 20B | nd^{(a)} | F | 3-CF₃-benzylamino | t-butoxy |
| 20C | nd^{(a)} | Br | 3-CF₃-benzylamino | t-butoxy |
| 20D | nd^{(a)} | Br | 3-CF₃-benzylamino | t-butoxy |
| 20E | (3S,4R) | H | (R)-1,2,3,4- | t-butoxy |
| | | | tetrahydronaphth-1- | |
| | | | ylamino | |
| 20F | (3S,4R) | H | 1-phenyl-cyclopent- | t-butoxy |
| | | | 1-ylamino | |
| 20G | (3S,4R) | H | t-butoxy | 3-CF₃-benzyl |

^{(a)}nd = not determined

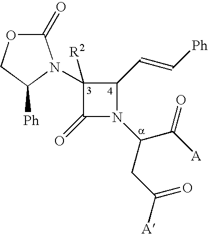

| Example | Stereochemistry | R² | A | A′ |
|---|---|---|---|---|
| 20H | (αS,3S,4R) | Me | N-methyl-3-CF₃- | t-butoxy |
| | | | benzylamino | |
| 20I | nd^{(a)} | Me | 3-CF₃-benzylamino | t-butoxy |
| 20J | (αS,3S,4R) | H | 1-phenyl-cyclopent-1- | t-butoxy |
| | | | ylamino | |
| 20K | (αS,3S,4R) | H | (R)-1-phenylethyl-1-amino | t-butoxy |
| 20L | (αR,3S,4R) | H | α,α-dimethylbenzylamino | t-butoxy |
| 20M | nd^{(a)} | H | N-methyl-3-CF₃- | t-butoxy |
| | | | benzylamino | |
| 20N | nd^{(a)} | H | (R)-α-methylbenzylamino | t-butoxy |
| 20O | (αR,3S,4R) | H | (R)-α,N- | t-butoxy |
| | | | dimethylbenzylamino | |
| 20P | (αS,3S,4R) | H | 4-cyclohexylpiperidinyl | t-butoxy |

^{(a)}nd = not determined

### Example 20Q

2(S)-(tert-Butoxycarbonylaminopropyl)-2-[3(S)-(4(S)-phenyloxazolidin-2-on-3-yl)-4(R)-(styr-2-yl)azetidin-2-on-1-yl]acetic acid N-(3-trifluoromethylbenzyl)amide. The imine prepared from L-ornithine γ-t-butoxycarbamate α-(3-trifluoromethyl)benzylamide and cinnamaldehyde was combined with 2-(4(S)-phenyloxazolidin-2-on-3-yl)acetyl chloride (Example 1A) to give the title compound.

### Example 20R

2(S)-(Aminopropyl)-2-[3(S)-(4(S)-phenyloxazolidin-2-on-3-yl)-4(R)-(styr-2-yl)azetidin-2-on-1-yl]acetic acid N-(3-trifluoromethylbenzyl)amide hydrochloride. Example 20Q was treated with trifluoroacetic acid in CH₂Cl₂ to give the title compound.

### Example 21

General procedure for hydrolysis of a tert-butyl ester. A solution of tert-butyl ester derivative in formic acid, generally 1 g in 10 mL, is stirred at ambient temperature until ester is not detected by thin layer chromatography (dichloromethane 95%/methanol 5%), generally about 3 hours. The formic acid is evaporated under reduced pressure; the resulting solid residue is partitioned between dichloromethane and saturated aqueous sodium bicarbonate. The organic layer is evaporated, generally to give a white or an off-white solid that may be used directly for further reactions, or recrystallized from an appropriate solvent system if desired.

### Examples 22

Were prepared from the corresponding tert-butyl ester according to the procedure used in Example 21.

### Example 22A

2(R,S)-(Carboxy)-2-[3(S)-(4(S)-phenyloxazolidin-2-on-3-yl)-4(R)-(2-styryl)azetidin-2-on-1-yl]acetic acid N-(3-trifluoromethylbenzyl)amide. Example 18A (0.30 g, 0.46 mmol) was hydrolyzed to give 0.27 g (quantitative yield) as an off-white solid; ¹H NMR (CDCl₃) δ 4.17-5.28 (m, 9H); 6.21-6.29 (m, 1H), 6.68-6.82 (m, 1H); 7.05-7.75 (m, 13H); 9.12-9.18 (m, 1H).

### Example 22B

2(S)-(Carboxymethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. Example 19A (1.72 g, 2.59 mmol) was hydrolyzed to give 1.57 g (quantitative yield) as an off-white solid; ¹H NMR (CDCl₃) δ 2.61 (dd, J=9.3 Hz, J=16.6 Hz, 1H); 3.09-3.14 (m, 1H); 4.10-4.13 (m, 1H); 4.30 (d, J=4.5 Hz, 1H); 4.39-4.85 (m, 6H); 6.20 (dd, J=9.6 Hz, J=15.7 Hz, 1H); 6.69 (d, J=15.8 Hz, 1H); 7.12-7.15 (m, 2H); 7.26-7.50 (m, 1H); 7.61 (s, 1H); 8.41-8.45 (m, 1H).

### Example 22C

2(S)-(Carboxymethyl)-2-[3(R)-(4(R)-phenyloxazolidin-2-on-3-yl)-4(S)-(2-styryl)azetidin-2-on-1-yl]acetic acid N-(3-trifluoromethylbenzyl)amide. Example 19B (41 mg, 0.06 mmol) was hydrolyzed to give 38 mg (quantitative yield) as an off-white solid; ¹H NMR (CDCl₃) δ 2.26 (d, J=7 Hz, 1H); 4.03 (t, J=7 Hz, 1H); 4.16 (t, J=8 Hz, 1H); 4.26 (d, J=4.3 Hz, 1H); 4.46 (dd, J=5.7 Hz, J=15.1, 1H); 4.53-4.75 (m, 5H); 6.25 (dd, J=9.5 Hz, J=15.7 Hz, 1H); 6.77 (d, J=15.7 Hz, 1H); 7.28-7.53 (m, 13H); 7.64 (s, 1H); 8.65-8.69 (m, 1H).

### Example 22D

2(S)-(Carboxyethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. Example 19C (4.97 g, 7.34 mmol) was hydrolyzed to give 4.43 g (97%) as an off-white solid; ¹H NMR (CDCl₃) δ 1.92-2.03 (m, 1H); 2.37-2.51 (m, 3H); 4.13-4.19 (m, 1H); 3.32 (d, J=4.9 Hz, 1H); 4.35-4.39 (m, 1H); 4.44 (dd, J=5.9 Hz, J=14.9 Hz, 1H); 4.50-4.57 (m, 2H); 4.61-4.67 (m, 1H); 4.70-4.76 (m, 1H); 6.24 (dd, J=9.6 Hz, J=15.8 Hz, 1H); 6.70 (d, J=15.8 Hz, 1H); 7.18-7.47 (m, 14H).

### Example 22E

2(S)-(Carboxymethyl)-2-[3(S)-(4(S)-phenyloxazolidin-2-on-3-yl)-4(R)-(2-styryl)azetidin-2-on-1-yl]acetic acid N-[4-(2-phenylethyl)]piperazinamide. Example 19D (1.88 g, 2.78 mmol) was hydrolyzed to give 1.02 g (60%) as an off-white solid; ¹H NMR (CDCl₃) δ 2.63 (dd, J=6.0 Hz, J=16.5 Hz, 1H); 2.75-2.85 (m, 1H); 3.00 (dd, J=8.2 Hz, J=16.6 Hz, 1H); 3.13-3.26 (m, 4H); 3.37-3.56 (m, 4H); 3.86-4.00 (m, 1H); 4.05-4.11 (m, 1H); 4.24 (d, J=5.0 Hz, 1H); 4.46-4.66 (m, 1H); 4.65-4.70 (m, 1H); 5.10-5.15 (m, 1H); 6.14 (dd, J=9.3 Hz, J=15.9 Hz, 1H); 6.71 (d, J=15.9 Hz, 1H); 7.22-7.41 (m, 15H); 12.02 (s, 1H).

### Example 22F

2(S)-(Carboxyethyl)-2-[3(S)-(4(S)-phenyloxazolidin-2-on-3-yl)-4(R)-(2-styryl)azetidin-2-on-1-yl]acetic acid N-[4-(2-phenylethyl)]piperazinamide. Example 19E (0.383 g, 0.55 mmol) was hydrolyzed to give 0.352 g (quantitative yield) as an off-white solid; ¹H NMR (CDCl₃) δ 1.93-2.01 (m, 1H); 2.07-2.36 (m, 61′); 2.82-2.90 (m, 1H); 3.00-3.20 (m, 4H); 3.36-3.54 (m, 4H); 3.74-3.82 (m, 1H); 4.06-4.11 (m, 1H); 4.29 (d, J=4.9 Hz, 1H); 4.33-4.46 (m, 2H); 4.50-4.58 (m, 2H); 4.67-4.72 (m, 1H); 4.95-5.00 (m, 1H); 6.18 (dd, J=9.2 Hz, J=16.0 Hz, 1H); 6.67 (d, J=15.9 Hz, 1H); 7.19-7.42 (m, 15H); 8.80 (brs, 1H).

### Example 22G

2(R)-(Carboxymethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. Example 19F (1.51 g, 2.27 mmol) was hydrolyzed to give 1.38 g (quantitative yield) as an off-white solid.

### Example 22H

2(R)-(Carboxymethyl)-2-[3(R)-(4(R)-phenyloxazolidin-2-on-3-yl)-4(S)-(2-styryl)azetidin-2-on-1-yl]acetic acid N-(3-trifluoromethylbenzyl)amide. Example 19G (550 mg, 0.83 mmol) was hydrolyzed to give 479 mg (95%) as an off-white solid. Example 32A exhibited an ¹H NMR spectrum consistent with the assigned structure.

### Example 22I

2(R)-(Carboxyethyl)-2-[3(S)-(4(S)-phenyloxazolidin-2-on-3-yl)-4(R)-(2-styryl)azetidin-2-on-1-yl]acetic acid N-(3-trifluoromethylbenzyl)amide. Example 19H (0.604 g, 0.89 mmol) was hydrolyzed to give 0.554 g (quantitative yield) as an off-white solid.

### Example 22J

2(S)-(Carboxyethyl)-2-[3(S)-(4(S)-phenyloxazolidin-2-on-3-yl)-4(R)-(2-styryl)azetidin-2-on-1-yl]acetic acid N-(4-cyclohexyl)piperazinamide. Example 191 (0.537 g, 0.80 mmol) was hydrolyzed to give 0.492 g (quantitative yield) as an off-white solid; ¹H NMR (CDCl₃) δ 1.09-1.17 (m, 1H); 1.22-1.33 (m, 2H); 1.40-1.47 (m, 2H); 1.63-1.67 (m, 1H); 1.85-1.90 (m, 2H); 1.95-2.00 (m, 1H); 2.05-2.15 (m, 3H); 2.20-2.24 (m, 1H); 2.30-2.36 (m, 1H); 2.85-2.93 (m, 1H); 3.25-3.33 (m, 1H); 3.36-3.46 (m, 2H); 3.81-3.87 (m, 1H); 4.08 (t, J=8.3 Hz, 1H); 4.28 (d, J=5.0 Hz, 1H); 4.33-4.56 (m, 4H); 4.70 (t, J=8.3 Hz, 1H); 4.83-4.91 (m, 1H); 6.17 (dd, J=9.1 Hz, J=15.9 Hz, M1); 6.67 (d, J=15.9 Hz, 1H); 7.25-7.44 (m, 10H); 8.22 (brs, 1H).

### Example 22K

2(S)-(2-(4-Cyclohexylpiperazinylcarbonyl)ethyl)-2-[3(S)-(4(S)-phenyloxazolidin-2-on-3-yl)-4(R)-(2-styryl)azetidin-2-on-1-yl]acetic acid. Example 19J (0.787 g, 1.28 mmol) was hydrolyzed to give 0.665 g (92%) as an off-white solid; ¹H NMR (CDCl₃) δ 1.05-1.13 (m, 1H); 1.20-1.40 (m, 5H); 1.60-1.64 (m, 1H); 1.79-1.83 (m, 2H); 2.00-2.05 (m, 2H); 2.22-2.44 (m, 3H); 2.67-2.71 (m, 1H); 2.93-3.01 (m, 4H); 3.14-3.18 (m, 1H); 3.38-3.42 (m, 1H); 3.48-3.52 (m, 1H); 3.64-3.69 (m, 1H); 4.06-4.14 (m, 2H); 4.34-4.43 (m, 2H); 4.56 (t, J=8.8 Hz, 1H); 4.73 (t, J=8.4 Hz, 1H); 6.15 (dd, J=9.1 Hz, J=16.0 Hz, 1H); 6.65 (d, J=16.0 Hz, 1H); 7.25-7.42 (m, 10H).

### Example 22L

2(R)-(Carboxymethyl)-2-[3(S)-(4(S)-phenyloxazolidin-2-on-3-yl)-4(R)-(2-styryl)azetidin-2-on-1-yl]acetic acid N-(2-fluoro-3-trifluoromethylbenzyl)carboxamide. Example 19K (0.26 g, 0.38 mmol) was hydrolyzed to give 0.238 g (quantitative yield) as an off-white solid; ¹H NMR (CDCl₃) δ 3.27 (d, J=7.2 Hz, 1H); 4.06 (t, J=7.2 Hz, 1H); 4.15 (t, J=8.1 Hz, 1H); 4.27 (d, J=4.8 Hz, 1H); 4.56-4.76 (m, 5H); 6.34 (dd, J=9.5 Hz, J=15.7 Hz, 1H); 6.80 (d, J=15.7 Hz, 1H); 7.06 (t, J=7.7 Hz, 1H); 7.31-7.54 (m, 12H); 8.58 (t, J=5.9 Hz, 1H).

### Example 22M

2(R)-(Carboxymethyl)-2-[3(S)-(4(S)-phenyloxazolidin-2-on-3-yl)-4(R)-(2-styryl)azetidin-2-on-1-yl]acetic acid N—[(S)-α-methylbenzyl]amide. Example 19L (0.215 g, 0.35 mmol) was hydrolyzed to give 0.195 g (quantitative yield) as an off-white solid; ¹H NMR (CDCl₃) δ 1.56 (d, J=7.0 Hz, 1H); 3.10 (dd, J=4.5 Hz, J=17.9 Hz, 1H); 3.18 (dd, J=9.8 Hz, J=17.9 Hz, 1H); 4.00 (dd, J=4.5 Hz, J=9.7 Hz, 1H); 4.14 (t, J=8.2 Hz, 1H); 4.26 (d, J=4.7 Hz, 1H); 5.02-5.09 (m, 1H); 6.41 (dd, J=9.4 Hz, J=15.8 Hz, 1H); 6.78 (d, J=15.8 Hz, 1H); 7.18 (t, J=7.3 Hz, 1H); 7.26-7.43 (m, 12H); 8.29 (d, J=8.2 Hz, 1H).

### Example 22N

2(R)-(Carboxymethyl)-2-[3(S)-(4(S)-phenyloxazolidin-2-on-3-yl)-4(R)-(2-styryl)azetidin-2-on-1-yl]acetic acid N—[(R)-α-methylbenzyl]amide. Example 19M (0.22 g, 0.35 mmol) was hydrolyzed to give 0.20 g (quantitative yield) as an off-white solid; ¹H NMR (CDCl₃) δ 1.59 (d, J=7.0 Hz, 1H); 3.25 (d, J=7.0 Hz, 2H); 3.92 (t, J=7.3 Hz, 1H); 4.15 (t, J=8.3 Hz, 1H); 4.26 (d, J=5.0 Hz, 1H); 4.52 (dd, J=4.8 Hz, J=9.3 Hz, 1H); 4.65 (t, J=8.8 Hz, 1H); 4.72 (t, J=8.3 Hz, 1H); 5.07-5.28 (m, 1H); 6.29 (dd, J=9.5 Hz, J=15.6 Hz, 1H); 6.71 (d, J=16.0 Hz, 1H); 7.20-7.43 (m, 13H); 8.31 (d, J=8.0 Hz, 1H).

### Example 22O

2(R)-(Carboxymethyl)-2-[3(S)-(4(S)-phenyloxazolidin-2-on-3-yl)-4(R)-(2-styryl)azetidin-2-on-1-yl]acetic acid N-methyl-N-(3-trifluoromethylbenzyl)amide. Example 19N (0.253 g, 0.37 mmol) was hydrolyzed to give 0.232 g (quantitative yield) as an off-white solid; ¹H NMR (CDCl₃) δ 3.07-3.15 (m, 4H); 4.13 (t, J=8.2 Hz, 1H); 4.30 (d, J=4.9 Hz, 1H); 4.46-4.78 (m, 5H); 5.23 (dd, J=4.6 Hz, J=9.7 Hz, 1H); 6.20 (dd, J=9.4 Hz, J=15.9 Hz, 1H); 6.73 (d, J=15.9 Hz, 1H); 7.25-7.43 (m, 15H).

### Example 22P

2(S)-(Carboxyethyl)-2-[3(S)-(4(S)-phenyloxazolidin-2-on-3-yl)-4(R)-(2-chlorostyr-2-yl)azetidin-2-on-1-yl]acetic acid N-(3-trifluoromethylbenzyl)amide. Example 190 (0.707 g, 0.99 mmol) was hydrolyzed to give 0.648 g (99%) as an off-white solid; ¹H NMR (CDCl₃) δ 2.22-2.28 (m, 2H); 2.49-2.64 (m, 2H); 4.09 (t, J=8.0 Hz, 1H); 4.25-4.62 (m, 6H); 4.87 (t, J=8.0 Hz, 1H); 6.88 (s, 1H); 7.25-7.66 (m, 15H).

### Example 22Q

2(R)-(Carboxymethyl)-2-[3(S)-(4(S)-phenyloxazolidin-2-on-3-yl)-4(R)-(2′-methoxystyr-2-yl)azetidin-2-on-1-yl]acetic acid N-(3-trifluoromethylbenzyl)amide. Example 19P (0.268 g, 0.39 mmol) was hydrolyzed to give 0.242 g (98%) as an off-white solid; ¹H NMR (CDCl₃) δ 3.26 (d, J=7.1 Hz, 1H); 3.79 (s, 3H); 4.14 (t, J=8.2 Hz, 1H); 4.25 (d, J=4.5 Hz, 1H); 4.51 (dd, J=5.9 Hz, J=15.5 Hz, 1H); 4.53-4.66 (m, 4H); 6.36 (dd, J=9.4 Hz, J=15.8 Hz, 1H); 8.88 (t, J=8.2 Hz, 1H); 6.70 (d, J=15.8 Hz, 1H); 7.18 (d, J=6.5 Hz, 1H); 7.25-7.48 (m, 10H); 7.48 (s, 1H); 8.66-8.69 (m, 1H).

### Example 22R

(2R)-(Benzyloxymethyl)-2-[3(S)-(4(S)-phenyloxazolidin-2-on-3-yl)-4(R)-(2-styryl)azetidin-2-on-1-yl]acetic acid. Example 19Q (0.16 g, 0.28 mmol) was hydrolyzed to give 0.144 g (quantitative yield) as an off-white solid; ¹H NMR (CDCl₃) δ 3.65 (dd, J=4.0 Hz, J=9.5 Hz, 1H); 3.82 (dd, J=5.5 Hz, J=9.5 Hz, 1H); 4.11 (dd, J=7.8 Hz, J=8.8 Hz, 1H); 4.33 (s, 2H); 4.50 (d, J=5.0 Hz, 1H); 4.57 (t, J=9.0 Hz, 1H); 4.67 (dd, J=4.0 Hz, J=5.0 Hz, 1H); 4.69 (dd, J=5.0 Hz, J=9.5 Hz, 1H); 4.75 (t, J=8.0 Hz, 1H); 6.17 (dd, J=9.3 Hz, J=15.8 Hz, 1H); 6.55 (d, J=16.0 Hz, 1H); 7.09-7.12 (m, 2H); 7.19-7.42 (m, 13H).

### Example 22S

2(S)-(2-(4-Cyclohexylpiperazinylcarbonyl)methyl)-2-[3 (S)-(4(S)-phenyloxazolidin-2-on-3-yl)-4(R)-(2-styryl)azetidin-2-on-1-yl]acetic acid. Example 19R (737 mg, 1.12 mmol) was hydrolyzed to give 640 mg (95%) as an off-white solid.

### Example 22T

3(R)-[3(S)-(4(S)-Phenyloxazolidin-2-on-3-yl)-3-methyl-4(R)-(styr-2-yl)azetidin-2-on-1-yl]-3-[(3-trifluoromethyl)phenylmethylaminocarbonyl]propanoic acid. Prepared according to Example 21, 120 mg (0.18 mmol) of Example 19S was hydrolyzed to give 108 mg (98%) as an off-white solid; ¹H NMR (CDCl₃) δ 1.22 (s, 3H); 3.25 (dd, J=3.5 Hz, J=18.0 Hz, 1H); 3.36 (dd, J=10.8 Hz, J=18.2 Hz, 1H); 4.01 (dd, J=4.0 Hz, J=10.5 Hz, 1H); 4.05 (dd, J=3.8 Hz, J=8.8 Hz, 1H); 4.33 (d, J=9.0 Hz, 1H); 4.44-4.51 (m, 3H); 4.61-4.66 (m, 1H); 4.73 (dd, J=3.8 Hz, J=8.8 Hz, 1H); 6.19 (dd, J=9.0 Hz, J=16.0 Hz, 1H); 6.74 (d, J=16.0 Hz, 1H); 7.22-7.54 (m, 13H); 7.65 (s, 1H).

### Example 22U

2(R)-(Carboxymethyl)-2-[3(S)-(4(S)-phenyloxazolidin-2-on-3-yl)-4(R)-(propen-1-yl)azetidin-2-on-1-yl]acetic acid N-(3-trifluoromethylbenzyl)amide. Prepared according to Example 21, 160 mg (0.27 mmol) of Example 19T was hydrolyzed to give 131 mg (90%) as an off-white solid. ¹H NMR (CDCl₃) δ 1.69 (dd, J=1 Hz, J=6.5 Hz, 3H); 3.23 (d, J=7 Hz, 1H); 3.93 (t, J=7.3 Hz, 1H); 4.14-4.20 (m, 3H); 4.29 (dd, J=5 Hz, J=9.5 Hz, 1H); 4.43 (dd, J=6 Hz, J=15 Hz, 1H); 4.61 (dd, J=6.5 Hz, J=15 Hz, 1H); 4.66-4.74 (m, 2H); 5.50-5.55 (m, 1H); 5.90-5.98 (m, 1H); 7.32-7.60 (m, 9H); 8.60-8.64 (m, 1H).

### Example 22V

2(R)-(Carboxylmethyl)-2-[3(S)-(4(S)-phenyloxazolidin-2-on-3-yl)-4(R)-(2-styryl)azetidin-2-on-1-yl]acetic acid N—[(R)-1-(3-trifluoromethylphenyl)ethyl]amide. Example 19×(166 mg, 0.24 mmol) was hydrolyzed to give 152 mg (quantitative yield) as an off-white solid.

### Example 22W

2(S)-(Methoxycarbonylethyl)-2-[3(S)-(4(S)-phenyloxazolidin-2-on-3-yl)-4(R)-(2-styryl)azetidin-2-on-1-yl]acetic acid. Example 19V (875 mg, 1.64 mmol) was hydrolyzed to give 757 mg (97%) as an off-white solid.

### Example 22X

2(R)-(Carboxylmethyl)-2-[3(S)-(4(S)-phenyloxazolidin-2-on-3-yl)-4(R)-(2-styryl)azetidin-2-on-1-yl]acetic acid N—[(S)-1-(3-trifluoromethylphenyl)ethyl]amide. Example 22Y (38.5 mg, 0.057 mmol) was hydrolyzed to give 35 mg (quantitative yield) as an off-white solid.

### Example 22Y

2(S)-(tert-Butoxycarbonylethyl)-2-[3(S)-(4(S)-phenyloxazolidin-2-on-3-yl)-4(R)-(2-styryl)azetidin-2-on-1-yl]acetic acid. Example 19U (97 mg, 0.18 mmol) was dissolved in methanol/tetrahydrofuran (2.5 mL/2 mL) and reacted with lithium hydroxide (0.85 mL of a 0.85M solution in water; 0.72 mmol) for 6 hours at room temperature. The reaction was diluted with 15 mL dichloromethane and aqueous hydrochloric acid (1M) was added until the pH of the aqueous layer reached 5 (as measured by standard pH paper). The organic layer was then separated and evaporated to dryness to give 84 mg (89%) as an off-white solid.

### Example 22Z

2(S)-(tert-Butoxycarbonylmethyl)-2-[3(S)-(4(S)-phenyloxazolidin-2-on-3-yl)-4(R)-(2-styryl)azetidin-2-on-1-yl]acetic acid. Example 19W (200 mg, 0.39 mmol) was hydrolyzed according to Example 22Y to give 155 mg (88%) as an off-white solid.

### Example 23A

2(R)-(2-(3-trifluoromethylbenzyl)aminocarbonyl)eth-1-yl)-2-[3(S)-(4(S)-phenyloxazolidin-2-on-3-yl)-4(R)-(2-styryl)azetidin-2-on-1-yl]acetic acid. Example 19Z (150 mg, 0.24 mmol) was hydrolyzed according to Example 22Y to give 143 mg (97%) as an off-white solid.

The following Examples 24 were prepared according to the processes described herein:

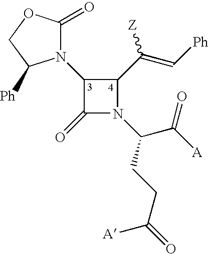

| Example | Stereochemistry | Y | A | A′ |
|---|---|---|---|---|
| 24A | (3S,4R) | F | 3-CF₃-benzylamino | OH |
| 24B | nd^{(a)} | F | 3-CF₃-benzylamino | OH |
| 24C | nd^{(a)} | Br | 3-CF₃-benzylamino | OH |
| 24D | nd^{(a)} | Br | 3-CF₃-benzylamino | OH |
| 24E | (3S,4R) | H | (R)-1,2,3,4-tetrahydro- | OH |
| | | | 1-naphtylamide | |
| 24F | (3S,4R) | H | 1-phenyl- | OH |
| | | | cyclopentylamide | |
| 24G | (3S,4R) | H | OH | 3-CF₃- |
| | | | | benzyl |

^{(a)}nd = not determined

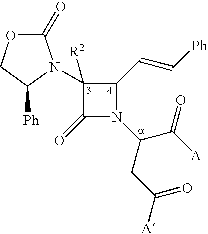

| Example | Stereochemistry | R² | A | A′ |
|---|---|---|---|---|
| 24H | (αS,3S,4R) | Me | N-methyl-3-CF₃-benzylamino | OH |
| 24I | nd^{(a)} | Me | 3-CF₃-benzylamino | OH |
| 24J | (αS,3S,4R) | H | 1-phenyl-cyclopent-1-ylamino | OH |
| 24K | (αS,3S,4R) | H | (R)-1-phenylethyl-1-amino | OH |
| 24L | (αR,3S,4R) | H | α,α-dimethylbenzylamino | OH |
| 24M | nd^{(a)} | H | N-methyl-3-CF₃-benzylamino | OH |
| 24N | nd^{(a)} | H | (R)-α-methylbenzylamino | OH |
| 24O | (αR,3S,4R) | H | (R)-α,N-dimethylbenzylamino | OH |
| 24P | (αS,3S,4R) | H | 4-cyclohexylpiperidinyl | OH |

^{(a)}nd = not determined

### Examples 25

Shown in the following Table, were prepared using the procedure of Example 6A, except that N-benzyloxycarbonyl-D-aspartic acid β-t-butyl ester monohydrate was replaced with Example 22A, and 3-(trifluoromethyl)benzyl amine was replaced with the appropriate amine.

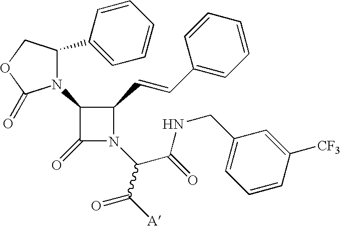

| Example | A′ |
|---|---|
| 25A | 2-(piperidinyl)ethylamino |
| 25B | 4-(piperidinyl)piperidinyl |
| 25C | 4-(2-phenylethyl)piperazinyl |
| 25D | 1-benzylpiperidin-4-ylamino |
| 25E | 4-butylpiperazinyl |
| 25F | 4-isopropylpiperazinyl |
| 25G | 4-cyclohexylpiperazinyl |
| 25H | 4-[2-(piperidinyl)ethyl]piperidinyl |

### Examples 26

Shown in the following Table, were prepared using the procedure of Example 6A, except that N-benzyloxycarbonyl-D-aspartic acid β-t-butyl ester monohydrate was replaced with Example 22B, and 3-(trifluoromethyl)benzyl amine was replaced with the appropriate amine.

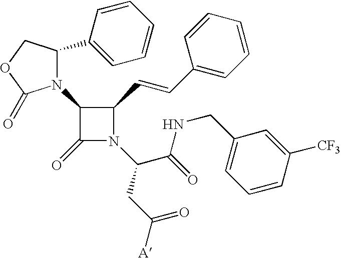

| Example | A′ |
|---|---|
| 26A | 2-(piperidinyl)ethylamino |
| 26B | 4-(piperidinyl)piperidinyl |
| 26C | 4-(phenylethyl)piperazinyl |
| 26D | fur-2-ylmethylamino |
| 26E | 4-(pyrrolidinyl)piperazinyl |
| 26F | 4-(3-trifluoromethylphenyl)piperazinyl |
| 26G | 4-(benzyloxycarbonyl)piperazinyl |
| 26H | 4-[2-(2-hydroxyethoxy)ethyl]piperazinyl |
| 26I | 4-benzylpiperazinyl |
| 26J | 4-(3,4-methylenedioxybenzyl)piperazinyl |
| 26K | 4-phenylpiperazinyl |
| 26L | 4-(3-phenylprop-2-enyl)piperazinyl |
| 26M | 4-ethylpiperazinyl |
| 26N | 2-(dimethylamino)ethylamino |
| 26O | 4-(pyrrolidinylcarbonylmethyl)piperazinyl |
| 26P | 4-(1-methylpiperidin-4-yl)piperazinyl |
| 26Q | 4-butylpiperazinyl |
| 26R | 4-isopropylpiperazinyl |
| 26S | 4-pyridylmethylamino |
| 26T | 3-(dimethylamino)propylamino |
| 26U | 1-benzylpiperidin-4-ylamino |
| 26V | N-benzyl-2-(dimethylamino)ethylamino |
| 26W | 3-pyridylmethylamino |
| 26X | 4-(cyclohexyl)piperazinyl |
| 26Y | 4-(2-cyclohexylethyl)piperazinyl |
| 26Z | 4-[2-(morpholin-4-yl)ethyl]piperazinyl |
| 26AA | 4-(4-tert-butylbenzyl)piperazinyl |
| 26AB | 4-[2-(piperidinyl)ethyl]piperazinyl |
| 26AC | 4-[3-(piperidinyl)propyl]piperazinyl |
| 6AD | 4-[2-(N,N-dipropylamino)ethyl]piperazinyl |
| 26AE | 4-[3-(N,N-diethylamino)propyl]piperazinyl |
| 26AF | 4-[2-(dimethylamino)ethyl]piperazinyl |
| 26AG | 4-[3-(pyrrolidinyl)propyl]piperazinyl |
| 26AH | 4-(cyclohexylmethyl)piperazinyl |
| 26AI | 4-cyclopentylpiperazinyl |
| 26AJ | 4-[2-(pyrrolidinyl)ethyl]piperazinyl |
| 26AK | 4-[2-(thien-2-yl)ethyl]piperazinyl |
| 26AL | 4-(3-phenylpropyl)piperazinyl |
| 26AM | 4-[2-(N,N-diethylamino)ethyl]piperazinyl |
| 26AN | 4-benzylhomopiperazinyl |
| 26AO | 4-(bisphenylmethyl)piperazinyl |
| 26AP | 3-(4-methylpiperazinyl)propylamino |
| 26AQ | (+)-3-(S)-1-benzylpyrrolidin-3-ylamino |
| 26AR | 2-pyridylmethylamino |
| 26AS | 4-[2-(piperidinyl)ethyl]piperidinyl |
| 26AT | 1-benzylpiperidin-4-ylamino N-oxide |

### Example 27

Example 26AU (44 mg, 0.06 mmol) was dissolved in 4 mL dichloromethane and reacted with 3-chloroperoxybenzoic acid (12 mg, 0.07 mmol) until the reaction was complete as determined by TLC (dichloromethane 94%/methanol 6%, UV detection). The reaction was quenched with aqueous sodium sulfite, the dichloromethane layer was washed with 5% aqueous sodium bicarbonate and distilled water. Evaporation of the dichloromethane layer gave an off-white solid (35 mg, 78%).

### Examples 28

Shown in The following table, were prepared using the procedure of Example 6A, except that N-benzyloxycarbonyl-D-aspartic acid β-t-butyl ester monohydrate was replaced with Example 22E, and 3-(trifluoromethyl)benzyl amine was replaced with the appropriate amine.

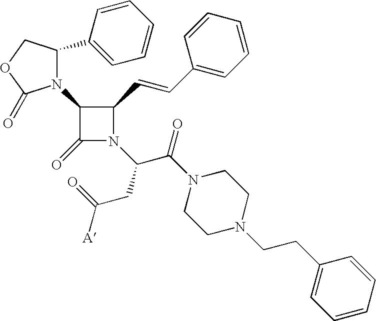

| Example | A′ |
|---|---|
| 28A | 3-trifluoromethylbenzylamino |
| 28B | morpholin-4-ylamino |
| 28C | 2-(dimethylamino)ethylamino |
| 28D | 3-(dimethylamino)propylamino |
| 28E | cyclohexylamino |
| 29F | piperidinyl |
| 28G | 2-methoxyethylamino |
| 28H | isopropylamino |
| 28I | isobutylamino |
| 28J | ethylamino |
| 28K | dimethylamino |
| 28L | methylamino |

### Examples 29

Shown in the following Table, were prepared using the procedure of Example 6A, except that N-benzyloxycarbonyl-D-aspartic acid β-t-butyl ester monohydrate was replaced with Example 22S, and 3-(trifluoromethyl)benzyl amine was replaced with the appropriate amine.

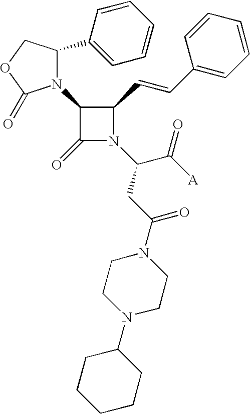

| Example | A |
|---|---|
| 29A | (2,3-dichlorobenzyl)amino |
| 29B | 1-phenylcyclohexylamino |

### Examples 30

Shown in the following Table were prepared according to the processes described herein.

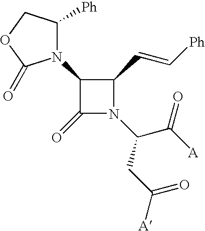

| Example | A | A′ |
|---|---|---|
| 30A | 1-phenyl-cyclopent-1-ylamino | 4-(piperidinyl)piperidinyl |
| 30B | 1-phenyl-cyclopent-1-ylamino | 1-benzylpiperidin-4-ylamino |
| 30C | (R)-1-phenylethyl-1-amino | 4-(piperidinyl)piperidinyl |

### Examples 31

Shown in the following Table, were prepared using the procedure of Example 6A, except that N-benzyloxycarbonyl-D-aspartic acid β-t-butyl ester monohydrate was replaced with Example 22G, and 3-(trifluoromethyl)benzyl amine was replaced with the appropriate amine. Example 31J was prepared using the procedure of Example 26AT, except that Example 31A was replaced with Example 35×, TO GIVE an off-white solid (48 mg, 94%).

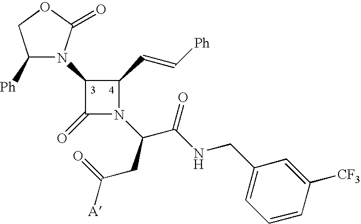

| Example | A′ |
|---|---|
| 31A | 4-(piperidinyl)piperidinyl |
| 31B | 4-(2-phenylethyl)piperazinyl |
| 31C | 4-[2-(piperidinyl)ethyl]piperidinyl |
| 31D | 4-(pyrrolidinyl)piperazinyl |
| 31E | 1-benzylpiperidin-4-ylamino |
| 31F | (pyridin-3-ylmethyl)amino |
| 31G | 3-(dimethylamino)propylamino |
| 31H | 3-(S)-(1-benzylpyrrolidin-3-yl)amino |
| 31I | 4-[(piperidinyl)methyl]piperidinyl |
| 31J | 4-(piperidinyl)piperidinyl N-oxide |

### Example 32

2(R)-[[4-(Piperidinyl)piperidinyl]carboxymethyl]-2-[3(S)-(4(R)-phenyloxazolidin-2-on-3-yl)-4(R)-(2-styryl)azetidin-2-on-1-yl]acetic acid N-(3-trifluoromethylbenzyl)amide. Prepared according to Example 6A, except that N-benzyloxycarbonyl-D-aspartic acid β-t-butyl ester monohydrate was replaced with Example 22H, and 3-(trifluoromethyl)benzyl amine was replaced with 4-(piperidinyl)piperidine.

### Examples 33

Shown in the following Table were prepared according to the processes described herein.

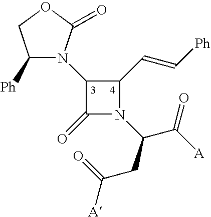

| Ex- | | | |
|---|---|---|---|
| am- | Stereo- | | |
| ple | chemistry | A | A′ |
| 33A | (3S, 4R) | α,α-dimethylbenzylamino | 4-(piperidinyl)piperidinyl |
| 33B | (3S, 4R) | α,α-dimethylbenzy1amino | 1-benzylpiperidin-4- |
| | | | ylamino |
| 33C | nd^{(a)} | N-methyl-3-CF3- | 4-(piperidinyl)piperidinyl |
| | | benzylamino | |
| 33D | (3S, 4R) | N-methyl-3-CF3- | 3- |
| | | benzylamino | (pyrrolidinyl)piperidinyl |
| 33E | nd^{(a)} | (R)-α-methylbenzylamino | 4-(piperidinyl)piperidinyl |
| 33F | (3S, 4R) | (R)-α,N- | 4-(piperidinyl)piperidinyl |
| | | dimethylbenzylamino | |

^{(a)}nd = not determined

### Example 33G

2(R)-[[4-(Piperidinyl)piperidinyl]carbonylmethyl]-2-[3 (S)-(4(S)-phenyloxazolidin-2-on-3-yl)-4(R)-(2-styryl)azetidin-2-on-1-yl]acetic acid N-(2-fluoro-3-trifluoromethylbenzyl)carboxamide. Prepared according to Example 6A, except that N-benzyloxycarbonyl-D-aspartic acid β-t-butyl ester monohydrate was replaced with Example 22L, and 3-(trifluoromethyl)benzyl amine was replaced with 4-(piperidinyl)piperidine.

### Example 33H

2(R)-[[4-(Piperidinyl)piperidinyl]carbonylmethyl]-2-[3 (S)-(4(S)-phenyloxazolidin-2-on-3-yl)-4(R)-(2-styryl)azetidin-2-on-1-yl]acetic acid N-[(S)-α-methylbenzyl]amide. Prepared according to Example 6A, except that N-benzyloxycarbonyl-D-aspartic acid β-t-butyl ester monohydrate was replaced with Example 22M, and 3-(trifluoromethyl)benzyl amine was replaced with 4-(piperidinyl)piperidine.

### Example 33I

2(R)-[[4-(Piperidinyl)piperidinyl]carbonylmethyl]-2-[3 (S)-(4(S)-phenyloxazolidin-2-on-3-yl)-4(R)-(2-styryl)azetidin-2-on-1-yl]acetic acid N-[(R)-α-methylbenzyl]amide. Prepared according to Example 6A, except that N-benzyloxycarbonyl-D-aspartic acid β-t-butyl ester monohydrate was replaced with Example 22N, and 3-(trifluoromethyl)benzyl amine was replaced with 4-(piperidinyl)piperidine.

### Example 33J

2(R)-[[4-[2-(piperidinyl)ethyl]piperidinyl]carbonylmethyl]-2-[3(S)-(4(S)-phenyloxazolidin-2-on-3-yl)-4(R)-(2-styryl)azetidin-2-on-1-yl]acetic acid N—[(S)-α-methylbenzyl]amide. Prepared according to Example 6A, except that N-benzyloxycarbonyl-D-aspartic acid β-t-butyl ester monohydrate was replaced with Example 22M, and 3-(trifluoromethyl)benzyl amine was replaced with 4-[2-(piperidinyl)ethyl]piperidine.

### Example 33K

2(R)-[[4-[2-(piperidinyl)ethyl]piperidinyl]carbonylmethyl]-2-[3(S)-(4(S)-phenyloxazolidin-2-on-3-yl)-4(R)-(2-styryl)azetidin-2-on-1-yl]acetic acid N—[(R)-α-methylbenzyl]amide. Prepared according to Example 6A, except that N-benzyloxycarbonyl-D-aspartic acid β-t-butyl ester monohydrate was replaced with Example 22N, and 3-(trifluoromethyl)benzyl amine was replaced with 4-[2-(piperidinyl)ethyl]piperidine.

### Example 33L

2(R)-[[4-(Piperidinyl)piperidinyl]carbonylmethyl]-2-[3 (S)-(4(S)-phenyloxazolidin-2-on-3-yl)-4(R)-(2-styryl)azetidin-2-on-1-yl]acetic acid N—[(R)-1-(3-trifluoromethylphenyl)ethyl]amide. Prepared according to Example 6A, except that N-benzyloxycarbonyl-D-aspartic acid β-t-butyl ester monohydrate was replaced with Example 22V, and 3-(trifluoromethyl)benzyl amine was replaced with 4-(piperidinyl)piperidine.

### Example 33M

2(R)-[[4-(Piperidinyl)piperidinyl]carbonylmethyl]-2-[3(S)-(4(S)-phenyloxazolidin-2-on-3-yl)-4(R)-(2-styryl)azetidin-2-on-1-yl]acetic acid N—[(S)-1-(3-trifluoromethylphenyl)ethyl]amide. Prepared according to Example 6A, except that N-benzyloxycarbonyl-D-aspartic acid β-t-butyl ester monohydrate was replaced with Example 22X, and 3-(trifluoromethyl)benzyl amine was replaced with 4-(piperidinyl)piperidine.

### Example 34A

2(R)-[[4-(Piperidinyl)piperidinyl]carbonylmethyl]-2-[3(S)-(4(S)-phenyloxazolidin-2-on-3-yl)-4(R)-(2-styryl)azetidin-2-on-1-yl]acetic acid N-methyl-N-(3-trifluoromethylbenzyl)amide. Prepared according to Example 6A, except that N-benzyloxycarbonyl-D-aspartic acid β-t-butyl ester monohydrate was replaced with Example 220, and 3-(trifluoromethyl)benzyl amine was replaced with 4-(piperidinyl)piperidine; Calc'd for C₄₃H₄₈F₃N₅O₅: C, 66.91; H, 6.27; N, 9.07; found. C, 66.68; H, 6.25; N, 9.01.

### Example 34B

Hydrochloride salt. Example 34A (212.5 mg) was dissolved in 30 mL dry Et₂O. Dry HCl gas was bubbled through this solution resulting in the rapid formation of an off-white precipitate. HCl addition was discontinued when no more precipitate was observed forming (ca. 5 minutes). The solid was isolated by suction filtration, washed twice with 15 mL of dry Et₂O and dried to 213.5 mg (96% yield) of an off-white solid; Calc'd for C₄₃H₄₉ClF₃N₅O₅: C, 63.89; H, 6.11; N, 8.66; Cl, 4.39; found. C, 63.41; H, 5.85; N, 8.60; Cl, 4.86.

### Examples 35

Shown in the following Table, were prepared according to Example 6A, except that N-benzyloxycarbonyl-D-aspartic acid β-t-butyl ester monohydrate was replaced with Example 22D, and 3-(trifluoromethyl)benzyl amine was replaced with the appropriate amine. Example 35AN was prepared using the procedure of Example 26AT, except that Example 26AU was replaced with Example 35× to give an off-white solid (54.5 mg, 98%). Example 35AO was prepared using the procedure of Example 6A, except that N-benzyloxycarbonyl-D-aspartic acid β-t-butyl ester monohydrate was replaced with Example 22W.

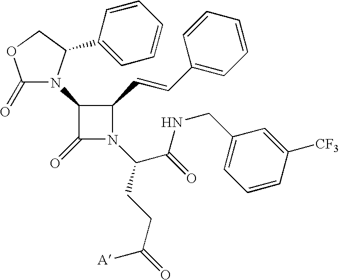

| Example | A′ |
|---|---|
| 35A | 2-(piperidinyl)ethylamino |
| 35B | 4-(piperidinyl)piperidinyl |
| 35C | 2-(pyrid-2-yl)ethylamino |
| 35D | morpholin-4-ylamino |
| 35E | 4-(pyrrolidinyl)piperazinyl |
| 35F | 4-(3-trifluorophenyl)piperazinyl |
| 35G | 4-(benzyloxycarbonyl)piperazinyl |
| 35H | 4-[2-(2-hydroxylethoxy)ethyl]piperazinyl |
| 35I | 4-benzylpiperazinyl |
| 35J | 4-(3,4-methylenedioxybenzyl)piperazinyl |
| 35K | 4-phenylpiperazinyl |
| 35L | 4-(3-phenylprop-2-enyl)piperazinyl |
| 35M | 4-ethylpiperazinyl |
| 35N | 2-(dimethylamino)ethylamino |
| 35O | 4-(pyrrolidinylcarbonylmethyl)piperazinyl |
| 35P | 4-(1-methylpiperidin-4-yl)piperazinyl |
| 35Q | 4-butylpiperazinyl |
| 35R | 4-isopropylpiperazinyl |
| 35S | 4-pyridylmethylamino |
| 35T | 3-(dimethylamino)propylamino |
| 35U | 1-benzylpiperidin-4-ylamino |
| 35V | N-benzyl-2-(dimethylamino)ethylamino |
| 35W | 3-pyridylmethylamino |
| 35X | 4-cyclohexylpiperazinyl |
| 35Y | 4-(2-cyclohexylethyl)piperazinyl |
| 35Z | 4-[2-(morpholin-4-yl)ethyl]piperazinyl |
| 35AA | 4-(4-tert-butylbenzyl)piperazinyl |
| 35AB | 4-[2-(piperidinyl)ethyl]piperazinyl |
| 35AC | 4-[3-(piperidinyl)propyl]piperazinyl |
| 35AD | 4-[2-(diisopropylamino)ethyl]piperazinyl |
| 35AE | 4-[3-(diethylamino)propyl]piperazinyl |
| 35AF | 4-(2-dimethylaminoethyl)piperazinyl |
| 35AG | 4-[3-(pyrrolidinyl)propyl]piperazinyl |
| 35AH | 4-(cyclohexylmethyl)piperazinyl |
| 35AI | 4-[2-(piperidinyl)ethyl]piperidinyl |
| 35AJ | 4-propyl-piperazinyl |
| 35AK | 4-[N-(isopropyl)acetamid-2-yl]piperazinyl |
| 35AL | 3-benzyl-hexahydro-(1H)-1,3-diazepinyl |
| 35AM | 4-(piperidinylmethyl)piperidinyl |
| 35AN | 4-cyclohexylpiperazinyl N-oxide |
| 35AO | methoxy |
| 35AP | 4-cyclohexylpiperazinyl |

### Example 36

2(S)-[4-(2-phenylethyl)piperazinyl-carbonylethyl]-2-[3(S)-(4(S)-phenyloxazolidin-2-on-3-yl)-4(R)-(2-styryl)azetidin-2-on-1-yl]acetic acid N-(3-trifluoromethylbenzyl)amide. Prepared according to Example 6A, except that N-benzyloxycarbonyl-D-aspartic acid β-t-butyl ester monohydrate was replaced with the carboxylic acid of Example 22D, and 3-(trifluoromethyl)benzyl amine was replaced with 4-(2-phenylethyl)piperazine; ¹H NMR (CDCl₃) δ 2.21-2.23 (m, 1H); 2.25-2.45 (m, 6H); 2.52-2.63 (m, 3H); 2.72-2.82 (m, 2H); 3.42-3.48 (m, 2H); 3.52-3.58 (m, 1H); 4.13-4.18 (m, 1H); 4.26 (dd, J=5.1 Hz, J=8.3 Hz, 1H); 4.29 (d, J=5.0 Hz, 1H); 4.44 (dd, J=6.0 Hz, J=15.0 Hz, 1H); 4.54 (dd, J=6.2 Hz, J=14.9 Hz, 1H); 4.61-4.68 (m, 2H); 4.70-4.75 (m, 1H); 6.27 (dd, J=9.6 Hz, J=15.8 Hz, 1H); 6.73 (d, J=15.8 Hz, 1H); 7.16-7.60 (m, 19H); 8.07-8.12 (m, 1H); FAB⁺ (M+H)+/z 794; Elemental Analysis calculated for C₄₅H₄₆F₃N₅O₅: C, 68.08; H, 5.84; N, 8.82. found: C, 67.94; H, 5.90; N, 8.64.

### Examples 37

Shown in the following Table, were prepared using the procedure of Example 6A, except that N-benzyloxycarbonyl-D-aspartic acid β-t-butyl ester monohydrate was replaced with Example 22J, and 3-(trifluoromethyl)benzyl amine was replaced with the appropriate amine.

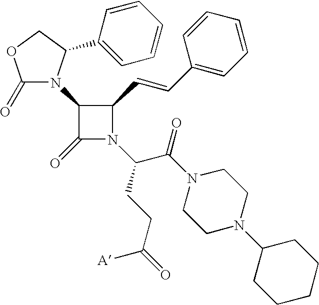

| Example | A′ |
|---|---|
| 37A | benzylamino |
| 37B | (2-methylbenzyl)amino |
| 37C | (3-methylbenzyl)amino |
| 37D | (4-methylbenzyl)amino |
| 37E | (α-methylbenzyl)amino |
| 37F | N-benzyl-N-methylamino |
| 37G | N-benzyl-N-(t-butyl)amino |
| 37H | N-benzyl-N-butylamino |
| 37I | (3,5-dimethylbenzyl)amino |
| 37J | (2-phenylethyl)amino |
| 37K | dimethylamino |
| 37L | (3-trifluoromethoxybenzyl)amino |
| 37M | (3,4-dichlorobenzyl)amino |
| 37N | (3,5-dichlorobenzyl)amino |
| 37O | (2,5-dichlorobenzyl)amino |
| 37P | (2,3-dichlorobenzyl)amino |
| 37Q | (2-fluoro-5-trifluoromethylbenzyl)amino |
| 37R | (4-fluoro-3-trifluoromethylbenzyl)amino |
| 37S | (3-fluoro-5-trifluoromethylbenzyl)amino |
| 37T | (2-fluoro-3-trifluoromethylbenzyl)amino |
| 37U | (4-chloro-3-trifluoromethylbenzyl)amino |
| 37V | indan-1-ylamino |
| 37W | 4-(2-hydroxybenzimidazol-1-yl)-piperidinyl |
| 37X | 3(S)-(tert-butylaminocarbonyl)-1,2,3,4- |
| | tetrahydroisoquinolin-2-yl |
| 37Y | (3,3-dimethylbutyl)amino |
| 37Z | 4-hydroxy-4-phenylpiperidinyl |
| 37AA | (cyclohexylmethyl)amino |
| 37AB | (2-phenoxyethyl)amino |
| 37AC | 3,4-methylenedioxybenzylamino |
| 37AD | 4-benzylpiperidinyl |
| 37AE | (3-trifluoromethylphenyl)amino |

### Examples 38

Shown in the following Table, were prepared using the procedure of Example 6A, except that N-benzyloxycarbonyl-D-aspartic acid β-t-butyl ester monohydrate was replaced with Example 22K, and 3-(trifluoromethyl)benzyl amine was replaced with the appropriate amine.

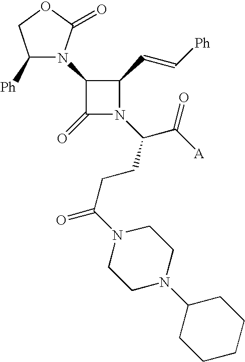

| Example | A |
|---|---|
| 38A | (3-trifluoromethoxybenzyl)amino |
| 38B | (3,4-dichlorobenzyl)amino |
| 38C | (3,5-dichlorobenzyl)amino |
| 38D | (2,5-dichlorobenzyl)amino |
| 38E | (2,3-dichlorobenzyl)amino |
| 38F | (2-fluoro-5-trifluoromethylbenzyl)amino |
| 38G | (4-fluoro-3-trifluoromethylbenzyl)amino |
| 38H | (3-fluoro-5-trifluoromethylbenzyl)amino |
| 38I | (2-fluoro-3-trifluoromethylbenzyl)amino |
| 38J | (4-chloro-3-trifluoromethylbenzyl)amino |
| 38K | (2-trifluoromethylbenzyl)amino |
| 38L | (3-methoxybenzyl)amino |
| 38M | (3-fluorobenzyl)amino |
| 38N | (3,5-difluorobenzyl)amino |
| 38O | (3-chloro-4-fluorobenzyl)amino |
| 38P | (3-chlorobenzyl)amino |
| 38Q | [3,5-bis(trifluoromethyl)benzyl]amino |
| 38R | (3-nitrobenzyl)amino |
| 38S | (3-bromobenzyl)amino |
| 38T | benzylamino |
| 38U | (2-methylbenzyl)amino |
| 38V | (3-methylbenzyl)amino |
| 38W | (4-methylbenzyl)amino |
| 38X | (α-methylbenzyl)amino |
| 38Y | (N-methylbenzyl)amino |
| 38Z | (N-tert-butylbenzyl)amino |
| 38AA | (N-butylbenzyl)amino |
| 38AB | (3,5-dimethylbenzyl)amino |
| 38AC | (2-phenylethyl)amino |
| 38AD | (3,5-dimethoxybenzyl)amino |
| 38AE | (1R)-(3-methoxyphenyl)ethylamino |
| 38AF | (1S)-(3-methoxyphenyl)ethylamino |
| 38AG | (α,α-dimethylbenzyl)amino |
| 38AH | N-methyl-N-(3-trifluoromethylbenzyl)amino |
| 38AI | [(S)-α-methylbenzyl]amino |
| 38AJ | (1-phenylcycloprop-1yl)amino |
| 38AK | (pyridin-2-ylmethyl)amino |
| 38AL | (pyridin-3-ylmethyl)amino |
| 38AM | (pyridin-4-ylmethyl)amino |
| 38AN | (fur-2-ylmethyl)amino |
| 38AO | [(5-methylfur-2-yl)methyl]amino |
| 38AP | (thien-2-ylmethyl)amino |
| 38AQ | [(S)-1,2,3,4-tetrahydro-1-naphth-1-yl]amino |
| 38AR | [(R)-1,2,3,4-tetrahydro-1-naphth-1-yl]amino |
| 38AS | (indan-1-yl)amino |
| 38AT | (1-phenylcyclopent-1-yl)amino |
| 38AU | (α,α-dimethyl-3,5-dimethoxybenzyl)amino |
| 38AV | (2,5-dimethoxybenzyl)amino |
| 38AW | (2-methoxybenzyl)amino |
| 38AX | (α,α,2-trimethylbenzyl)amino |
| 38AY | N-methyl-3-Me-benzylamide |
| 38AZ | N-methyl-2,3-Cl-benzylamide |
| 38BA | N-methyl-3-Cl-benzylamide |
| 38BB | N-methyl-3-Br-benzylamide |
| 38BC | N-methyl-3,5-Cl-benzylamide |
| 38BD | (R)-1-(3-trifluorophenyl)ethylamide |
| 38BE | 1-phenyl-cyclohexylamide |
| 38BF | 1-(2-fluorophenyl)-cyclopentylamide |
| 38BG | 1-(4-fluorophenyl)-cyclopentylamide |
| 38BH | 4-CF3-benzylamide |
| 38BI | α-phenyl-benzylamide |
| 38BJ | 3-phenyl-benzylamide |
| 38BK | dibenzylamide |
| 38BL | 1-naphthalene-methylamide |
| 38BM | 1,2,3,4-tetrahydro-isoquinolinamide |
| 38BN | indan-2-ylamino |
| 38BO | α-(2-OH-ethyl)benzylamide |
| 38BP | (S)-indan-1-ylamino |

### Examples 41

Shown in the following Table were prepared according to the processes described herein.

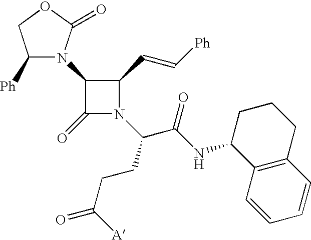

| Example | A′ |
|---|---|
| 41A | 4-(2-morpholinoethyl)-piperazinyl |
| 41B | 2-dimethylaminoethylamino |
| 41C | 4-(2-phenylethyl)-homopiperazinyl |
| 41D | 2-(1-piperidyl)ethylamino |
| 41E | (S)-2-(1-pyrrolidinylmethyl)pyrrolidinyl |
| 41F | 2-(1-pyrrolidinyl)ethylamino |
| 41G | 4-(1-piperidyl)piperidinyl |
| 41H | (R)-1-benzylpyrrolidin-3-ylamino |
| 41I | quinuclidin-3-ylamino |
| 41J | 4-methylhomopiperazinyl |
| 41K | 2-pyrrolylphenylamino |
| 41L | morpholin-4-ylethylamino |
| 41M | (S)-1-ethylpyrrolidin-2-ylaminomethyl |
| 41N | (R)-1-ethylpyrrolidin-2-ylaminomethyl |
| 41O | (S)-1-butoxycarbonylpyrrolidin-3-ylamino |
| 41P | quinolin-3-ylamino |
| 41Q | 4-(2-fluorophenyl)piperazinyl |
| 41R | 4-(3-chlorophenyl)piperazinyl |
| 41S | 4-(4-fluorophenyl)piperazinyl |
| 41T | 4-ethylpiperazinyl |
| 41U | 4-phenylpiperazinyl |
| 41V | 4-benzylpiperazinyl |
| 41W | 4-methylpiperazinyl |
| 41X | 4-(2-methoxyphenyl)piperazinyl |
| 41Y | 4-(3-OH-n-propyl)piperazinyl |
| 41Z | 4-(4-hydroxyphenyl)piperazinyl |
| 41AA | 3-(4-chlorophenyl)isoxazolin-5-ylamino |

### Examples 42

Shown in the following Table were prepared according to the processes described herein.

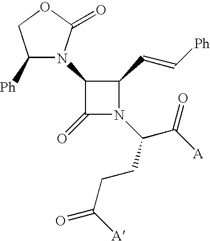

| Example | A | A′ |
|---|---|---|
| 42A | (R)-1-indanylamino | 4-cyclohexylpiperazinyl |
| 42B | 4-indanylamino | 4-cyclohexylpiperazinyl |
| 42C | 5-indanylamino | 4-cyclohexylpiperazinyl |
| 42D | indolinyl | 4-cyclohexylpiperazinyl |
| 42E | (αR)-α-(t- | 4-cyclohexylpiperazinyl |
| | butoxycarbonyl- | |
| | methyl)benzylamino | |
| 42F | 3-CF₃-benzylamino | 4-n-butylpiperazinyl |
| 42G | 3-CF₃-benzylamino | 4-ethylpiperazinyl |
| 42H | 1-(3-fluorophenyl)- | 4-cyclohexylpiperazinyl |
| | cyclopentylamino | |
| 42I | 1-(4-chlorophenyl)- | 4-cyclohexylpiperazinyl |
| | cyclopropylamino | |
| 42J | 1-(4-chlorophenyl)- | 4-cyclohexylpiperazinyl |
| | cyclopentylamino | |
| 42K | 1-(4-methylphenyl)- | 4-cyclohexylpiperazinyl |
| | cyclopropylamino | |
| 42L | 1-(4-methylphenyl)- | 4-cyclohexylpiperazinyl |
| | cyclopentylamino | |
| 42M | 1-(4-methoxyphenyl)- | 4-cyclohexylpiperazinyl |
| | cyclopropylamino | |
| 42N | 1-phenylcyclopentylamino | 4-(1-pyrrolidyl)piperidinyl |
| 42O | 1-phenylcyclopentylamino | 4-[3-((R)-Boc-amino)-1- |
| | | pyrrolidyl)piperidinyl |
| 42P | 1-phenylcyclopentylamino | (3R)-4-(3- |
| | | chloroammoniumpyrrolidinyl) |
| | | piperdinyl |

### Examples 44

Shown in the following Table, were prepared using the procedure of Example 6A, except that N-benz yloxycarbonyl-D-aspartic acid β-t-butyl ester monohydrate was replaced with Example 22I, and 3-(trifluoromethyl)benzyl amine was replaced with the appropriate amine.

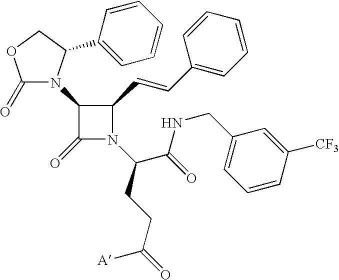

| Example | A | A′ |
|---|---|---|
| 44A | 3-CF₃-benzyl | 4-(piperidinyl)piperidinyl |
| 44B | 3-CF₃-benzyl | 4-(2-phenylethyl)piperazinyl |
| 44C | 3-CF₃-benzyl | 4-butylpiperazinyl |
| 44D | 3-CF₃-benzyl | 4-isopropylpiperazinyl |
| 44E | 3-CF₃-benzyl | 4-cyclohexylpiperazinyl |
| 44F | 3-CF₃-benzyl | 4-(cyclohexylmethyl)piperazinyl |
| 44G | 4-cyclohexylpiperazinyl | 3-CF₃-benzyl |

### Examples 45

Shown in the following Table, were prepared using the procedure of Example 6A, except that N-benzyloxycarbonyl-D-aspartic acid β-t-butyl ester monohydrate was replaced with Example 22P, and 3-(trifluoromethyl)benzyl amine was replaced with the appropriate amine.

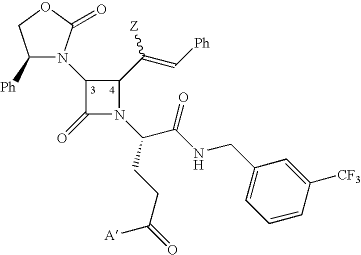

| Ex- | | | |
|---|---|---|---|
| am- | | | |
| ple | Z | Stereochemistry | A′ |
| 45A | Cl | (3S, 4R) | 4-cyclohexylpiperazinyl |
| 45B | Cl | (3S, 4R) | 4-(pyrrolidinyl)piperazinyl |
| 45C | Cl | (3S, 4R) | 4-[2-(2-hydroxyethyloxy)ethyl]piperazinyl |
| 45D | Cl | (3S, 4R) | 4-benzylpiperazinyl |
| 45E | Cl | (3S, 4R) | 4-(3,4-methylendioxybenzyl)piperazinyl |
| 45F | Cl | (3S, 4R) | 4-ethylpiperazinyl |
| 45G | Cl | (3S, 4R) | 4-n-butylpiperazinyl |
| 45H | Cl | (3S, 4R) | 4-isopropylpiperazinyl |
| 45I | Cl | (3S, 4R) | 1-benzylpiperidin-4-ylamino |
| 45J | Cl | (3S, 4R) | 4-(2-cyclohexylethyl)piperazinyl |
| 45K | Cl | (3S, 4R) | 4-[2-(morpholin-4-yl)ethyl]piperazinyl |
| 45L | F | nd^{(a)} | 4-n-butylpiperazinyl |
| 45M | F | nd^{(a)} | (R)-1-benzylpyrrolidin-3-amino |
| 45N | F | nd^{(a)} | quinuclidin-3-ylamino |
| 45O | F | (3S, 4R) | (S)-1-benzylpyrrolidin-3-amino |
| 45P | Cl | nd^{(a)} | (R)-1-benzylpyrrolidin-3-amino |
| 45Q | Cl | (3S, 4R) | (R)-1-benzylpyrrolidin-3-amino |
| 45R | Cl | (3S, 4R) | (S)-1-benzylpyrrolidin-3-amino |
| 45S | Cl | nd^{(a)} | (S)-1-benzylpyrrolidin-3-amino |
| 45T | Br | nd^{(a)} | 4-n-butylpiperazinyl |
| 45U | Br | nd^{(a)} | 4-ethylpiperazinyl |

^{(a)}nd = not determined

### Example 46

2(S)-[[4-(Piperidinyl)piperidinyl]carbonymethyl]-2-[3(S)-(4(R)-phenyloxazolidin-2-on-3-yl)-4(R)-(2-styryl)azetidin-2-on-1-yl]acetic acid N-(3-trifluoromethylbenzyl)amide. Prepared according to Example 6A, except that N-benzyloxycarbonyl-D-aspartic acid β-t-butyl ester monohydrate was replaced with Example 22C, and 3-(trifluoromethyl)benzyl amine was replaced with 4-(piperidinyl)piperidine.

### Example 47

2(R)-[[4-(Piperidinyl)piperidinyl]carbonylmethyl]-2-[3 (S)-(4(S)-phenyloxazolidin-2-on-3-yl)-4(R)-(1-(2-methoxyphenyl)ethen-2-yl)azetidin-2-on-1-yl]acetic acid N-(3-trifluoromethylbenzyl)amide. Prepared according to Example 6A, except that N-benzyloxycarbonyl-D-aspartic acid β-t-butyl ester monohydrate was replaced with Example 22Q, and 3-(trifluoromethyl)benzyl amine was replaced with 4-(piperidinyl)piperidine.

### Example 49

tert-Butyl[3(S)-(4(S)-phenyloxazolidin-2-on-3-yl)-4(R)-(2-styryl)azetidin-2-on-1-yl]acetate. Prepared according to Example 14, the imine prepared from 4.53 g (34.5 mmol) glycine tert-butyl ester and cinnamaldehyde was combined with 2-(4(S)-phenyloxazolidin-2-on-3-yl)acetyl chloride (Example 1A) to give 5.5 g (30%) as colorless crystals (recrystallized, n-chlorobutane); mp 194-195° C.

### Example 50

General procedure for α-alkylation and/or acylation of an (azetidin-2-on-1-yl)acetate. A solution of (azetidin-2-on-1-yl)acetate in tetrahydrofuran (ca. 0.22 M in azetidinone), such as Example 49, is cooled to −78° C. and treated with lithium bis(trimethylsilyl)amide (2.2 equivalents). The resulting anion is treated with an appropriate alkyl or acyl halide (1.1 equivalents). Upon complete conversion of the azetidinone, the reaction is quenched with saturated aqueous ammonium chloride and partitioned between ethyl acetate and water. The organic phase is washed sequentially with 1N HCl, saturated aqueous sodium bicarbonate, and saturated aqueous NaCl. The resulting organic layer is dried (e.g. magnesium sulfate) and evaporated. The residue is purified by silica gel chromatography with an appropriate eluent, such as 3:2 hexane/ethyl acetate.

This procedure may be used to prepare additional compounds of formulae described herein by an alternate synthetic route from a common intermediate such as tert-Butyl[3(S)-(4(S)-phenyloxazolidin-2-on-3-yl)-4(R)-(2-styryl)azetidin-2-on-1-yl]acetate, and related compounds. This procedure is also used to prepare alkylated and acylated analogs of the compounds described herein, such as compounds of formulae (I), (II), and (III) wherein R¹ is other than hydrogen. It is further appreciated that this procedure may be modified to introduce additional groups onto the azetidinone ring to prepare compounds described herein where R² is other than hydrogen.

### Example 51

(2RS)-[4-(piperidinyl)piperidinylcarbonyl]-2-[3(S)-(4(S)-phenyloxazolidin-2-on-3-yl)-4(R)-(2-styryl)azetidin-2-on-1-yl]propanoic acid N-(3-trifluoromethylbenzyl)amide. Example 25B (50 mg, 0.067 mmol) in tetrahydrofuran (4 mL) was treated sequentially with sodium hydride (4 mg, 0.168 mmol) and methyl iodide (6 μL, 0.094 mmol) at −78° C. The resulting mixture was slowly warmed to ambient temperature, and evaporated. The resulting residue was partitioned between dichloromethane and water, and the organic layer was evaporated. The resulting residue was purified by silica gel chromatography (95:5 chloroform/methanol) to give 28 mg (55%) as an off-white solid; MS (ES⁺): m/z=757 (M⁺).

The compounds shown in the following Table were prepared according to the processes described herein.

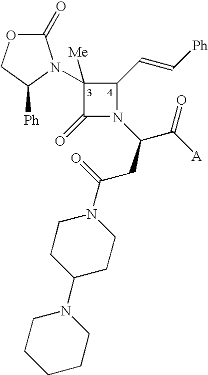

| | | | FAB |
|---|---|---|---|
| Example | Stereochemistry | A | (m + H)⁺ |
| 52A | (3S, 4R) | 3-CF₃-benzylamino | 772 |
| 52B | (3S, 4R) | N-methyl-3-CF₃-benzylamino | — |
| 52C | nd^{(a)} | 3-CF₃-benzylamino | — |

^{(a)}nd = not determined

### Example 53A

2(S)-[[(1-Benzylpiperidin-4-yl)amino]carbonylmethyl]-2-[3 (S)-(4(S)-phenyloxazolidin-2-on-3-yl)-4(R)-(2-phenyleth-1-yl)azetidin-2-on-1-yl]acetic acid N-(3-trifluoromethylbenzyl)amide. Prepared according to Example 9A, except that N-benzyloxycarbonyl-L-aspartic acid β-t-butyl ester α-(3-trifluoromethyl)benzylamide was replaced with Example 26U (50 mg, 0.064 mmol) to give 40 mg (80%) as an off-white solid.

### Example 53B

(2S)-[(4-cyclohexylpiperazinyl)carbonylethyl]-2-[3 (S)-(4(S)-phenyloxazolidin-2-on-3-yl)-4(R)-(2-phenyleth-1-yl)azetidin-2-on-1-yl]acetic acid N-(3-trifluoromethylbenzyl)amide. Prepared according to Example 9A, except that N-benzyloxycarbonyl-L-aspartic acid β-t-butyl ester α-(3-trifluoromethyl)benzylamide was replaced with Example 35×(50 mg, 0.065 mmol) to give 42 mg (84%) as an off-white solid.

### Example 53C

(2S)-[(4-cyclohexylpiperazinyl)carbonylethyl]-2-[3(S)-(4(S)-phenyloxazolidin-2-on-3-yl)-4(R)-(2-phenyleth-1-yl)azetidin-2-on-1-yl]acetic acid N—[(R)-1,2,3,4-tetrahydronaphth-1-yl]amide. Prepared according to Example 9A, except that N-benzyloxycarbonyl-L-aspartic acid β-t-butyl ester α-(3-trifluoromethyl)benzylamide was replaced with Example 38AR (76 mg, 0.10 mmol) to give 69 mg (90%) as an off white solid.

### Example 53D

2(R)-[[4-(Piperidinyl)piperidinyl]carbonylmethyl]-2-[3 (S)-(4(S)-phenyloxazolidin-2-on-3-yl)-4(R)-(propen-1-yl)azetidin-2-on-1-yl]acetic acid N-(3-trifluoromethylbenzyl)amide. Prepared according to Example 6A, except that N-benzyloxycarbonyl-D-aspartic acid β-t-butyl ester monohydrate was replaced with Example 22U, and 3-(trifluoromethyl)benzyl amine was replaced with 4-(piperidinyl)piperidine.

### Examples 62

Shown in the following Table, were prepared using the procedure of Example 6A, except that N-benzyloxycarbonyl-D-aspartic acid β-t-butyl ester monohydrate was replaced with Example 22R, and 3-(trifluoromethyl)benzyl amine was replaced with the appropriate amine.

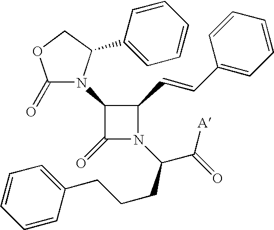

| Example | A′ | α |
|---|---|---|
| 62A | 4-(piperidinyl)piperidinyl | D |
| 62B | (3-trifluorobenzyl)amino | D |
| 62C | 4-(3-trifluoromethylphenyl)piperazinyl | D or L |
| 62D | 4-(3-trifluoromethylphenyl)piperazinyl | D or L |
| 62E | 4-cyclohexylpiperazinyl | DL |
| 62F | 4-(piperidinylmethyl)piperidinyl | D |
| 62G | 4-[2-(piperidinyl)ethyl]piperidinyl | D |
| 62H | 4-[(1-piperidyl)methyl]piperidinamide | D |

### Example 63

(S)-(benzyl)-D-cysteine-[4-(2-(1-piperidyl)ethyl)]piperidinamide Dihydrochloride. N-t-Butoxycarbonyl-(S)-(benzyl)-D-cysteine-[4-(2-(1-piperidyl)ethyl)]piperidinamide (0.453 g, 0.93 mmole) was reacted overnight with acetyl chloride (0.78 mL, 13.80 mmole) in anhydrous methanol (15 mL) to give an off-white solid by evaporating the reaction mixture to dryness (0.417 g, 97%). ¹H NMR (CD₃OD) δ 0.94-1.29 (m, 2H); 1.49-1.57 (m, 1H); 1.62-1.95 (m, 10H); 2.65-2.80 (m, 2H); 2.81-2.97 (m, 4H); 3.01-3.14 (m, 2H); 3.50-3.60 (m, 3H); 3.81-3.92 (m, 2H); 4.41-4.47 (m, 2H); 7.25-7.44 (m, 5H).

### Example 64A

(2S)-(Benzylthiomethyl)-2-[3(S)-(4(S)-phenyloxazolidin-2-on-3-yl)-4(R)-(2-styryl)azetidin-2-on-1-yl]acetic acid N-[4-[2-(piperid-1-yl)ethyl]piperidin-1-yl]amide. The imine prepared from (S)-(benzyl)-D-cysteine-[4-(2-(1-piperidyl)ethyl)]piperidinenamide, dihydrochloride (Example 63, 0.417 g, 0.90 mmole) and cinnamaldehyde, in the presence on triethylamine (0.26 mL, 1.87 mmole), was combined with 2-(4(S)-phenyloxazolidin-2-on-3-yl)acetyl chloride (Example 1A) according to the procedure of Example 14 to give 0.484 g (76%) as an off-white solid after recrystallization from dichloromethane/hexanes. ¹H NMR (CDCl₃) δ 0.89-1.06 (m, 2H); 1.40-1.44 (m, 5H); 1.57-1.67 (m, 6H); 2.25-2.43 (m, 6H); 2.45-2.59 (m, 2H); 2.71-2.88 (m, 2H); 3.55-3.70 (m, 3H); 4.11-4.17 (m, 1H); 4.37-4.47 (m, 2H); 4.54-4.61 (m, 1H); 4.64-4.69 (m, 1H); 4.76-4.84 (m, 2H); 6.05-6.19 (m, 1H); 6.66-6.71 (m, 1H); 7.12-7.40 (m, 15H).

### Examples 65, 66, and 67

Shown in the following Table, may also be prepared using the procedures described herein by replacing the serine or cysteine derivative described above with the one corresponding to the compounds shown below.

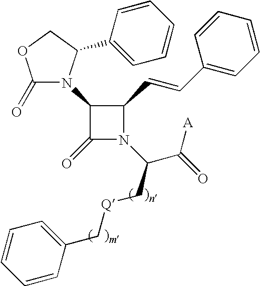

| Ex- | | | | |
|---|---|---|---|---|
| ample | A | n′ | Q′ | m′ |
| 65A | (3-trifluorobenzyl)amino | 2 | —O— | 1 |
| 65B | 4-(3-trifluoromethylphenyl)piperazin-1-yl | 2 | —O— | 2 |
| 65C | 4-(3-trifluoromethylphenyl)piperazin-1-yl | 2 | —O— | 1 |
| 65D | 4-cyclohexylpiperazin-1-yl | 2 | —O— | 2 |
| 65E | 4-(piperidin-1-ylmethyl)piperidin-1-yl | 2 | —O— | 1 |
| 65F | 4-(piperidin-1-yl)piperidin-1-yl | 2 | —O— | 2 |
| 65G | 4-[2-(piperidin-1-yl)ethyl]piperidin-1-yl | 2 | —O— | 1 |
| 66A | (3-trifluorobenzyl)amino | 1 | —S— | 2 |
| 66B | 4-(3-trifluoromethylphenyl)piperazin-1-yl | 1 | —S— | 1 |
| 66C | 4-(3-trifluoromethylphenyl)piperazin-1-yl | 1 | —S— | 2 |
| 66D | 4-cyclohexylpiperazin-1-yl | 1 | —S— | 1 |
| 66E | 4-(piperidin-1-ylmethyl)piperidin-1-yl | 1 | —S— | 2 |
| 66F | 4-(piperidin-1-yl)piperidin-1-yl | 1 | —S— | 1 |
| 66G | 4-[2-(piperidin-1-yl)ethyl]piperidin-1-yl | 1 | —S— | 2 |
| 66H | (3-trifluorobenzyl)amino | 2 | —S— | 2 |
| 66I | 4-(3-trifluoromethylphenyl)piperazin-1-yl | 2 | —S— | 1 |
| 66J | 4-(3-trifluoromethylphenyl)piperazin-1-yl | 2 | —S— | 2 |
| 66K | 4-cyclohexylpiperazin-1-yl | 2 | —S— | 1 |
| 66L | 4-(piperidin-1-ylmethyl)piperidin-1-yl | 2 | —S— | 2 |
| 66M | 4-(piperidin-1-yl)piperidin-1-yl | 2 | —S— | 1 |
| 66N | 4-[2-(piperidin-1-yl)ethyl]piperidin-1-yl | 2 | —S— | 2 |
| 67A | (3-trifluorobenzyl)amino | 0 | —CH₂— | 1 |
| 67B | 4-(3-trifluoromethylphenyl)piperazin-1-yl | 0 | —CH₂— | 2 |
| 67C | 4-(3-trifluoromethylphenyl)piperazin-1-yl | 0 | —CH₂— | 1 |
| 67D | 4-cyclohexylpiperazin-1-yl | 0 | —CH₂— | 2 |
| 67E | 4-(piperidin-1-ylmethyl)piperidin-1-yl | 0 | —CH₂— | 1 |
| 67F | 4-(piperidin-1-yl)piperidin-1-yl | 0 | —CH₂— | 2 |
| 67G | 4-[2-(piperidin-1-yl)ethyl]piperidin-1-yl | 0 | —CH₂— | 1 |

### Example 70A

Benzyl 2-[3(S*)-2-Phenyl)-4(R*)-(2-styryl)azetidin-2-on-1-yl]acetate. The imine prepared from glycine benzyl ester and cinnamaldehyde was combined with 2-phenylacetyl chloride according to Example 14.

### Example 70B

Benzyl 2-[3(S*)-2-Thienyl)-4(R*)-(2-styryl)azetidin-2-on-1-yl]acetate. The imine prepared from glycine benzyl ester and cinnamaldehyde was combined with 2-thiophene acetyl chloride according to Example 14.

### Example 70C

Benzyl 2(RS)-tert-Butylcarbonyl-2-[3(S*)-2-thienyl)-4(R*)-(2-styryl)azetidin-2-on-1-yl]acetate. Prepared from Example 70B by deprotonation and acylation with pivaloyl chloride.

### Example 70D

2(R)-(tert-Butoxycarbonylmethyl)-2-[3(S*)-2-thienyl)-4(R*)-(2-styryl)azetidin-2-on-1-yl]acetic acid N-(3-trifluoromethylbenzyl)amide. The imine prepared from 290 mg (0.84 mmol) of D-aspartic acid β-t-butyl ester α-(3-trifluoromethyl)benzylamide and cinnamaldehyde was combined with 2-thiophene-acetyl chloride according to Example 14 to give 42 mg (8%) after flash column chromatography purification (70:30 hexanes/ethyl acetate).

The following compounds are described

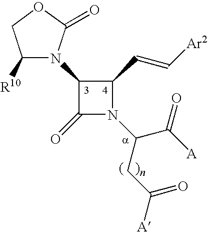

| Example | R¹⁰ | Ar² | n | α | A | A′ |
|---|---|---|---|---|---|---|
| 71A | Ph | 3- | 2 | L | (R)-1,2,3,4- | 4-cyclohexyl- |
| | | MeO- | | | tetrahydronaphth- | piperazin-1-yl |
| | | Ph | | | 1-ylamino | |
| 71B | Ph | 3- | 2 | L | 1-phenyl- | 4-cyclohexyl- |
| | | MeO- | | | cyclopent-1- | piperazin-1-yl |
| | | Ph | | | 1-ylamino | |
| 71C | Ph | 3- | 2 | L | (R)-1,2,3,4- | 4-cyclohexyl- |
| | | MeS- | | | tetrahydronaphth- | piperazin-1-yl |
| | | Ph | | | 1-ylamino | |
| 71D | Ph | 3-Cl- | 2 | L | (R)-1,2,3,4- | 4-cyclohexyl- |
| | | Ph | | | tetrahydronaphth- | piperazin-1-yl |
| | | | | | 1-ylamino | |
| 71E | Ph | 3-Cl- | 2 | L | 1-phenyl- | 4-cyclohexyl- |
| | | Ph | | | cyclopent-1- | piperazin-1-yl |
| | | | | | ylamino | |
| 71F | Ph | 3-F- | 2 | L | (R)-1,2,3,4- | 4-cyclohexyl- |
| | | Ph | | | tetrahydronaphth- | piperazin-1-yl |
| | | | | | 1-ylamino | |
| 71G | Ph | 3-Cl- | 1 | D | N-methyl-3-CF₃- | 4-(1- |
| | | Ph | | | benzylamino | piperidyl) |
| | | | | | | piperidinamide |
| 71H | Ph | 3- | 2 | L | (R)-1,2,3,4- | 4-cyclohexyl- |
| | | CF₃O- | | | tetrahydronaphth- | piperazin-1-yl |
| | | Ph | | | 1-ylamino | |
| 71I | 3- | 3-Cl | 2 | L | (R)-1,2,3,4- | 4-cyclohexyl- |
| | Cl- | Ph | | | tetrahydronaphth- | piperazin-1-yl |
| | Ph | | | | 1-ylamino | |
| 71J | Ph | 3,5- | 2 | L | (R)-1,2,3,4- | 4-cyclohexyl- |
| | | Cl₂- | | | tetrahydronaphth- | piperazin-1-yl |
| | | Ph | | | 1-ylamino | |

The following compounds are described

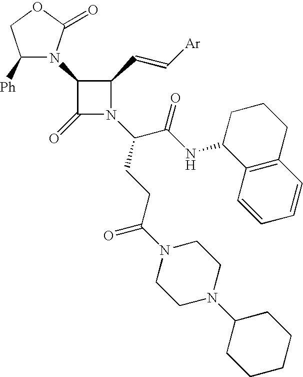

| Example | Ar |
|---|---|
| 72A | benzothiophen-7-yl |
| 72B | fur-2-yl |
| 72C | thien-2-yl |

The following compounds are described

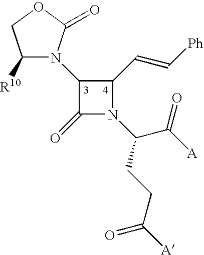

| Example | R¹⁰ | Stereochemistry | A | A′ |
|---|---|---|---|---|
| 73A | Ph | (3S, 4R) | (R)-1,2,3,4-tetrahydronaphth-1- | 4-cycloheptylpiperazin-1-yl |
| | | | ylamino | |
| 73B | Ph | (3S, 4R) | (R)-1,2,3,4-tetrahydronaphth-1- | 4-(tetrahydrothiopyran-4- |
| | | | ylamino | yl)piperazin-1-yl |
| 73C | Ph | (3R, 4S) | 3-CF₃-benzylamino | 4-cyclohexylpiperazin-1-yl |
| 73D | Ph | (3S, 4R) | 4-phenylpiperazin-1-yl | 3-F-5-CF₃-benzylamino |
| 73E | Ph | (3S, 4R) | 4-(2-cyclohexylethyl)piperazin- | 3-F-5-CF₃-benzylamino |
| | | | 1-yl | |
| 73F | Ph | (3S, 4R) | 4-(pyrid-2-yl)piperazin-1-yl | 3-F-5-CF₃-benzylamino |
| 73G | Ph | (3S, 4R) | 4-(2-thien-2-ylethyl)piperazin-1- | 3-F-5-CF₃-benzylamino |
| | | | yl | |
| 73H | 3-Cl- | (3S, 4R) | (R)-α-methylbenzylamino | 4-cyclohexylpiperazin-1-yl |
| | Ph | | | |

The following table illustrates selected compounds further characterized by mass spectral analysis using FAB⁺ to observe the corresponding (M+H)⁺ parent ion.

| Example | (m + H)⁺/z |
|---|---|
| 25B | 744 |
| 25C | 766 |
| 25D | 766 |
| 25E | 718 |
| 25F | 704 |
| 25G | 744 |
| 25H | 772 |
| 26B | 758 |
| 26U | 780 |
| 26AQ | 766 |
| 26AS | 786 |
| 29B | 758 |
| 31A | 758 |
| 31C | 786 |
| 31E | 780 |
| 31I | 772 |
| 33G | 776 |
| 33I | 704 |
| 34A | 772 |
| 35B | 772 |
| 35E | 759 |
| 35I | 780 |
| 35J | 824 |
| 35R | 732 |
| 35X | 772 |
| 35Y | 800 |
| 35Z | 803 |
| 35AH | 786 |
| 35AI | 800 |
| 35AJ | 732 |
| 35AN | 788 |
| 38C | 772 |
| 38D | 772 |
| 38E | 772 |
| 38F | 790 |
| 38H | 790 |
| 38I | 790 |
| 38K | 772 |
| 38L | 734 |
| 38M | 722 |
| 38N | 740 |
| 38O | 756 |
| 38P | 738 |
| 38Q | 840 |
| 38R | 749 |
| 38S | 782 |
| 38T | 704 |
| 38U | 718 |
| 38V | 718 |
| 38AB | 732 |
| 38AC | 718 |
| 38AD | 764 |
| 38AE | 748 |
| 38AF | 748 |
| 38AH | 786 |
| 38AI | 718 |
| 38AJ | 730 |
| 38AK | 705 |
| 38AL | 705 |
| 38AM | 705 |
| 38AN | 694 |
| 38AO | 708 |
| 38AP | 710 |
| 38AQ | 744 |
| 38AR | 744 |
| 38AS | 7530 |
| 38AT | 758 |
| 38AU | 792 |
| 38AV | 764 |
| 38AW | 734 |
| 38AX | 746 |
| 44B | 794 |
| 44C | 746 |
| 44D | 732 |
| 44E | 772 |
| 45A | 806 |
| 45B | 792 |
| 45F | 752 |
| 45G | 780 |
| 45H | 766 |
| 46 | 758 |
| 47 | 788 |
| 51 | 758 |
| 53A | 782 |
| 53B | 774 |
| 62A | 663 |
| 62G | 691 |

What is claimed is:

1. A compound of the formula: 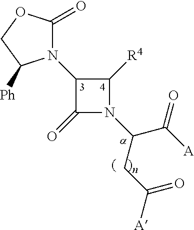
and pharmaceutically acceptable salts thereof, wherein
R⁴ is substituted phenylethenyl;
n is an integer selected from 1 and 2;
A is 1,2,3,4-tetrahydronaphth-1-ylamino; and
A′ is —OH or —NH₂; or A′ is taken together with the attached carbonyl group to form an ester or an amide.

2-4. (canceled)

5. The compound of claim 1 wherein A′ is a 4-substituted piperidinyl or 4-substituted piperazinyl.

6. The compound of claim 1 wherein the phenylethenyl is substituted with one or more substituents selected from the group consisting of F, Cl, OMe, SMe, NO₂, CN, CF₃, and OCF₃.

7. A method for treating a disease or disorder responsive to reduced signaling by the cannabinoid-1 receptor, the cannabinoid-2 receptor, or a combination thereof, the method comprising the step of administering to a patient in need of relief from the disease or disorder a composition comprising a pharmaceutically acceptable carrier, excipient, or diluent, or a combination thereof; and a compound in an amount effective to treat the disease or disorder, where the compound is of the formula: 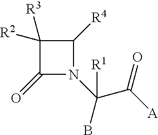
and enantiomers, diastereomers, and stereoisomeric mixtures thereof, and pharmaceutically acceptable salts thereof, wherein:
A is —OH or —NH₂; or A is taken together with the attached carbonyl group to form an ester or an amide;
B is a carboxylic acid, or an ester or amide derivative thereof, or B is alkyl, arylalkyl, hydroxyalkyl, alkylthiol, arylhydroxyalkyl, arylalkylthiol, aminoalkyl, or acyl, each of which is optionally substituted, or a derivative thereof, including ethers, esters, amides, carbonates, carbamates, ureas, ketals, and the like;
R¹ is hydrogen or C₁-C₆ alkyl;
R² is hydrogen, alkyl, alkenyl, alkynyl, alkoxy, alkylthio, halo, haloalkyl, cyano, formyl, alkylcarbonyl, or a substituent selected from the group consisting of —CO₂R⁸, —CONR⁸R^{8′}, and —NR⁸(COR⁹); where R⁸ and R^{8′} are each independently selected from hydrogen, alkyl, cycloalkyl, optionally substituted aryl, or optionally substituted arylalkyl; or R⁸ and R^{8′} are taken together with the attached nitrogen atom to form a heterocyclyl group;
and where R⁹ is selected from hydrogen, alkyl, cycloalkyl, alkoxyalkyl, optionally substituted aryl, optionally substituted arylalkyl, optionally substituted heteroaryl, optionally substituted heteroarylalkyl, and R⁸R^{8′}N—(C₁-C₄ alkyl);
R³ is an amino, amido, acylamido, or ureido group, which is optionally substituted; or R³ is a nitrogen-containing heterocyclyl group attached at a nitrogen atom; or R³ is an optionally substituted aryl group;
R⁴ is alkyl, alkenyl, alkynyl, cycloalkyl, cycloalkenyl, alkylcarbonyl, optionally substituted aryl, optionally substituted arylalkyl, optionally substituted arylhaloalkyl, optionally substituted arylalkoxyalkyl, optionally substituted arylalkenyl, optionally substituted arylhaloalkenyl, or optionally substituted arylalkynyl;
R⁸ and R^{8′} are each independently selected from hydrogen, alkyl, including C₁-C₆ alkyl, cycloalkyl, including C₃-C₈ cycloalkyl, optionally substituted aryl, or optionally substituted arylalkyl, including aryl(C₁-C₄ alkyl); or R⁸ and R^{8′} are taken together with the attached nitrogen atom to form an heterocycle, such as optionally substituted pyrrolidinyl, piperidinyl, morpholinyl, piperazinyl, and homopiperazinyl; and
R⁹ is selected from hydrogen, alkyl, including C₁-C₆ alkyl, cycloalkyl, including C₃-C₈ cycloalkyl, alkoxyalkyl, including (C₁-C₄ alkoxy)-(C₁-C₄ alkyl), optionally substituted aryl, optionally substituted arylalkyl, including aryl(C₁-C₄ alkyl), optionally substituted heteroaryl, optionally substituted heteroarylalkyl, including heteroaryl(C₁-C₄ alkyl), and R⁸R^{8′}N—(C₁-C₄ alkyl).

8. The method of claim 7 wherein the disease or disorder is selected from the group consisting of obesity, eating disorders, cravings, and addictive disorders.

9. The method of claim 7 wherein the disease or disorder is selected from the group consisting of bone disease, osteoporosis, allergy and allergic reaction, asthma, immune disorder, inflammation, and renal ischemia.

10. The method of claim 7 wherein the compound is of the formula: 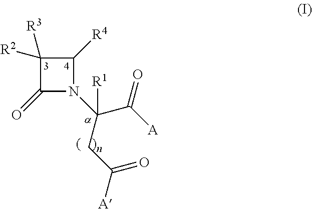
and pharmaceutically acceptable salts thereof, wherein
A is —OH and —NH₂; or A is taken together with the attached carbonyl group to form an ester or an amide;
A′ is —OH and —NH₂; or A′ is taken together with the attached carbonyl group to form an ester or an amide; and
n is an integer selected from 0 to about 3.

11-12. (canceled)

13. The method of claim 10 wherein A′ is piperidinyl or piperazinyl, or piperidinylamino or piperazinylamino, substituted at the 4-position.

14-16. (canceled)

17. The method of claim 7 wherein the compound is of the formula: 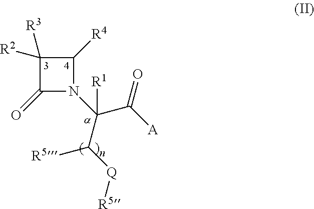
and pharmaceutically acceptable salts thereof; wherein
A is —OH or —NH₂; or A is taken together with the attached carbonyl group to form an ester or an amide;
Q is oxygen; or Q is sulfur or disulfide, or an oxidized derivative thereof;
n is an integer from 1 to 3;
R^{5″} is selected from hydrogen, alkyl, cycloalkyl, alkoxyalkyl, optionally substituted arylalkyl, optionally substituted heterocyclyl or optionally substituted heterocyclylalkyl, and optionally substituted aminoalkyl; and
R^{5′″} is selected from hydrogen, alkyl, and optionally substituted arylalkyl.

18. (canceled)

19. The method of claim 17 wherein R⁵ is hydrogen or lower alkyl; and R^{5′″} is alkyl or optionally substituted arylalkyl.

20. The method of claim 7 wherein the compound is of the formula: 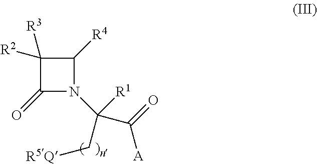
and pharmaceutically acceptable salts thereof; wherein
n is an integer in the range from about 1 to about 5, and is illustratively 1, 2, or 3;
A is —OH or —NH₂; or A is taken together with the attached carbonyl group to form an ester or an amide;
Q′ is oxygen or sulfur;
R^{5′} is selected from the group consisting of C₁-C₆ alkyl, C₃-C₈ cycloalkyl, (C₁-C₄ alkoxy)-(C₁-C₄ alkyl), optionally-substituted aryl(C₁-C₄ alkyl), Y′—(C₁-C₄ alkyl), where Y′- is a heterocycle, and R^{6′}R^{7′}N—(C₂-C₄ alkyl); where Y′ is selected from the group consisting of tetrahydrofuryl, morpholinyl, pyrrolidinyl, piperidinyl, piperazinyl, homopiperazinyl, or quinuclidinyl; where said morpholinyl, pyrrolidinyl, piperidinyl, piperazinyl, homopiperazinyl, or quinuclidinyl is optionally N-substituted with C₁-C₄ alkyl or optionally-substituted aryl(C₁-C₄ alkyl);
R^{6′} is hydrogen or alkyl, including C₁-C₆ alkyl, and R^{7′} is alkyl, including C₁-C₆ alkyl, cycloalkyl, including C₃-C₈ cycloalkyl, optionally substituted aryl, or optionally substituted arylalkyl, including aryl(C₁-C₄ alkyl); or R^{6′} and R^{7′} are taken together with the attached nitrogen atom to form an heterocycle, such as pyrrolidinyl, piperidinyl, morpholinyl, piperazinyl, and homopiperazinyl; where said piperazinyl or homopiperazinyl is optionally N-substituted with R^{13′}; and
R^{13′} is selected from hydrogen, alkyl, including C₁-C₆ alkyl, cycloalkyl, including C₃-C₈ cycloalkyl, alkoxycarbonyl, including C₁-C₄ alkoxycarbonyl, optionally substituted aryloxycarbonyl, optionally substituted arylalkyl, including aryl(C₁-C₄ alkyl), and optionally substituted aryloyl.

21. The method of an), one of claim 7
wherein the compound is of the formula: 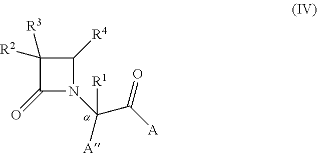
and pharmaceutically acceptable salts thereof; wherein
A is —OH or —NH₂; or A is taken together with the attached carbonyl group to form an ester or an amide; and
A″ is hydrogen, alkyl, cycloalkyl, aryl, heteroaryl, arylalkyl, heteroarylalkyl, aminoalkyl or a derivative thereof, alkylcarbonyl, cycloalkylcarbonyl, arylcarbonyl, heteroarylcarbonyl, arylalkylcarbonyl, or heteroarylalkylcarbonyl, each of which may be optionally substituted; and where the carbonyl of each is optionally an alkylene, arylalkylene, or heteroarylalkylene ketal.

22-23. (canceled)

24. The method of claim 21 wherein A″ is hydrogen, A is alkoxy or arylalkoxy, each of which is optionally substituted, and R³ is optionally substituted aryl.

25. The method of claim 21 wherein A″ is an alkyl, arylalkyl, or heteroarylalkyl corresponding to a naturally occurring aminoacid side chain.

26. The method of claim 21 wherein A″ is an alkylcarbonyl, arylalkylcarbonyl, or heteroarylalkylcarbonyl.

27-29. (canceled)

30. The method of claim 21 wherein A″ is aminopropyl or a carbamate, amide, or urea derivative thereof.

31. (canceled)

32. The method of claim 7 wherein the compound is of the formula: 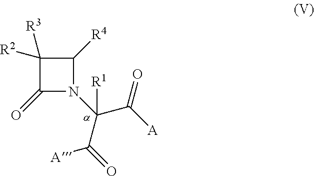
and pharmaceutically acceptable salts thereof; wherein
A is a —CO₂H, or an ester or an amide derivative thereof; and
A′″ is alkyl, cycloalkyl, aryl, heteroaryl, arylalkyl, or heteroarylalkyl, each of which may be optionally substituted, or A′″ and the attached carbonyl are taken together to form an alkylene, arylalkylene, or heteroarylalkylene ketal derivative, each of which may be optionally substituted.

33. The method of claim 32 wherein A′″ is branched alkyl.

34. (canceled)

35. The method of claim 32 wherein both R¹ and R² are hydrogen.

36. The method of claim 32 wherein R³ is optionally substituted aryl.

37-85. (canceled)
